(19) United States
(12) Patent Application Publication
(10) Pub. No.: US 2003/0083299 A1
(43) Pub. Date: May 1, 2003

(54) Non-invasive delivery of polypeptides through the blood-brain barrier

(76) Inventor: Ian A. Ferguson, Adelaide (AU)

Correspondence Address: Patrick D. Kelly, 11939 Manchester Rd. #403, St. Louis, MO, 63131, US

(21) Appl. No.: 10/188,184

(22) Filed: Jul. 2, 2002

Related U.S. Application Data
(63) Continuation-in-part of application No. 09/705,428, filed on Nov. 4, 2000, now abandoned.

(51) Int. Cl. A61K 48/00; C12N 15/85

(52) U.S. Cl. 514/44; 435/455

(57) ABSTRACT

A treatment method and genetic vectors are disclosed for non-invasive delivery of polypeptides through the blood brain barrier (BBB), to treat brain or spinal tissue. A genetic vector is used to transfect one or more neurons which “straddle” the BBB, such as sensory neurons, nocioceptive neurons, or lower motor neurons; this is done by administering the vector in a manner that causes it to contact neuronal projections that extend outside the BBB. Once inside a peripheral projection that belongs to a BBB-straddling neuron, the vectors (or some portion thereof) will be transported to the main cell body of the neuron, through a process called retrograde transport. Inside the main cell body, at least one gene carried by the genetic vector will be expressed, to form polypeptides. Some of these polypeptides (which can include leader sequences that will promote anterograde transport and secretion by BBB-straddling neurons) will be transported by the neurons to secretion sites inside the BBB. The polypeptides will be secreted by transfected neurons at locations inside the BBB, and will then contact and exert their effects upon secondary “target” neurons located entirely within the BBB. By using this system, polypeptides that stimulate nerve growth or activity can be used to treat neurodegenerative diseases, impaired limbs in stroke victims, etc., and polypeptides that suppress neuronal activity can be used to treat unwanted excessive neuronal activity, such as neuropathic pain. This approach also provides new methods for delivering endocrine and paracrine polypeptides into the CNS, thereby allowing improved medical and reproductive treatments in humans, and improved ability to modulate growth, maturation, reproduction, or other endocrine-related functions among livestock, endangered species, and other animals.

### FIELD OF INVENTION

This invention relates to methods for delivering polypeptides into central nervous system (CNS) tissue in humans and other mammals, and in other animals that have blood-brain barriers, including reptiles and birds. It also relates to methods for targeting delivery of polypeptides to specific populations or types of neurons that reside entirely within brain or spinal cord tissue. This invention further relates to medical treatments for various brain, spinal, or other neurological disorders in humans, and to other forms of treatment (such as to regulate fertility, maturation, growth rates, etc.) in other types of animals, using polypeptides that normally cannot cross the BBB.

### BACKGROUND OF THE INVENTION

As is well known to physicians and researchers, the “blood brain barrier” (BBB) in higher animals helps ensure that neurons inside the brain and spinal cord are not exposed to molecules which are proper and natural when present in circulating blood, but which would interfere with proper neuronal functioning if they penetrate the BBB. As used herein, the phrase “higher animal” is used to include any type of animal which has a blood-brain barrier.

As just one example of why the BBB is necessary, glutamic acid (also known as glutamate, the ionized form which exists at physiological pH) is an amino acid used by all cells for creating proteins; it is essential for cell growth and maintenance, and it circulates freely in blood. However, inside the brain and spinal cord, glutamate is a potent neurotransmitter; indeed, it is one of the most important excitatory neurotransmitters used by neurons to transmit nerve signals to each other. Therefore, if blood-borne glutamate could freely penetrate through capillary walls inside the CNS, it would trigger random and uncontrollable nerve signals that would cripple the brain's ability to generate and process coherent thoughts, memories, and other CNS activities.

Therefore, the brains of higher animals (including reptiles, birds, and mammals) developed in ways that caused the endothelial cells which form capillary walls inside the brain and spinal cord to form “tight junctions”, in which the membranes of adjacent endothelial cells are almost fused together. By contrast, endothelial cells which form capillary walls outside the brain and spinal cord have “slit-pores” between them, which make non-CNS capillaries much more permeable than capillaries inside the CNS.

This system, and the various types and classes of molecules which can and cannot pass through the BBB, are described in nearly any textbook on physiology or neurology, and in numerous review articles (e.g., Goldstein et al 1986; Pardridge 1998; Rubin et al 1999; Banks 1999; Kniesel et al 2000; Kniesel et al 2000). Animal models for studying BBB permeation are described in Bonate 1995, and early efforts to develop in vitro (cell culture) models are described in de Boer et al 1999.

Although the BBB system is essential for protecting the brain and spinal cord from unwanted disruptions and interference, it excludes numerous types of drugs and therapeutic agents that might be able to do a tremendous amount of good for people suffering from various types of diseases or injuries that affect the brain and/or spinal cord. In particular, for the purposes of this invention, it is important to recognize that, with only a very few exceptions, proteins and peptides cannot and do not cross the BBB in any substantial quantities (e.g., Langer 1990). The known exceptions to this rule mainly involve active transport systems (as described in Kastin et al 1999), or certain receptors located on the surfaces of neurons, such as transferrin receptors (e.g., Granholm et al 1998). Under the current art, those very minor and limited exceptions cannot provide adequate methods for allowing therapeutic treatment of the CNS using numerous types of potentially very useful polypeptides.

The pressing need for better and more effective methods of transporting therapeutic polypeptides (and other types of useful drugs) across the BBB is well-known, and numerous review articles have been published on that subject, citing and summarizing literally hundreds of other research articles. Such review articles include Zlokovic 1995, Cloughesy et al 1995, Davis et al 1995, Abbott et al 1996, Begley 1996, Kroll et al 1998, Pardridge et al 1998, Rochat et al 1999, and Rapoport 2000.

This invention relates to the transport of proteins, or polypeptides, across the BBB. Before proceeding further in that analysis, several points of terminology need to be clarified. As used herein, the terms polypeptides and proteins are used interchangeably herein. For any reader not familiar with these terms, a peptide bond is the type of chemical bond that is formed when two amino acids are bonded to each other, in the manner which occurs naturally in polypeptides and protein molecules. Accordingly, “peptide” is a broad term, which includes any molecule that contains at least one peptide bond.

Under an old and classical definition, the term polypeptide merely indicates that a large (or at least substantial) number of amino acids have been coupled together, to form a relatively large molecule having numerous peptide bonds. However, there is no numerical boundary line that clearly divides “polypeptides” from smaller peptides, and some important peptides have as few as 5, 6, or 7 amino acids.

To avoid confusion in the absence of any clear definition of how many amino acid residues must be present in a “poly”peptide, the term “polypeptide” is used herein in a slightly different manner. As used herein, the term “polypeptide” refers to a peptide molecule which has been formed, inside a living cell, by a process of gene expression. As is well known, gene expression inside cells involves the standard steps of (i) transcription of a DNA gene sequence to form a complementary strand of messenger RNA (mRNA), followed by (ii) translation of the mRNA strand, mainly by ribosomes, to form a polypeptide chain made of amino acids that have been bonded together in a precise sequence. Any such molecule formed by expression of a gene sequence is referred to herein as a polypeptide, regardless of whether the polypeptide is subsequently processed by one or more “post-translation” steps such as cleavage, cell secretion, glycosylation, cysteine crosslinking, etc.

Some researchers limit the term “protein” to describe polypeptides that are complete and fully functional, as distinct from broken polypeptide fragments, or polypeptide precursors that have not yet been processed in ways necessary to render them functional. For the purposes of this invention, a polypeptide will not be of interest unless it is indeed capable of carrying out an intended function; therefore, the terms “polypeptide” and “protein” are used interchangeably herein.

A polypeptide is of interest herein (and is covered by the claims herein) only if it has three traits. First: as discussed in more detail below, the polypeptide must be expressed by a foreign gene (also called an “exogenous” gene) which has been inserted into one or more classes of neurons, using an intervention method such as described herein. It should be noted, however, that a foreign (exogenous) gene carried by a vector may have the same sequence as a “native” or “natural” gene that is expressed normally inside the CNS of a patient or animal; this might occur, for example, in a treatment to boost levels of a certain polypeptide which is no longer being produced in adequate amounts by a person suffering from a disease.

Second: the polypeptide must be expressed in one or more classes of neurons which are then capable of releasing the polypeptide inside brain or spinal tissue which normally is protected by the blood-brain barrier. As used herein, phrases such as “delivering polypeptides through a blood-brain barrier” or “releasing polypeptides inside (or into) brain or spinal tissue” indicate and require that the foreign polypeptide(s) must contact at least one cell or class of cells that reside wholly within the BBB. This requirement is not satisfied if a polypeptide is secreted by a transfected BBB-straddling neuron only at locations where it contacts other BBB-straddling neurons. Instead, in most situations, this requirement will be satisfied if and when a foreign polypeptide is secreted by a transfected BBB-straddling neuron, at one or more synaptic junctions or other neuronal termini, into cerebrospinal fluid (CSF) and/or synaptic fluid, at a location inside the BBB.

Third: a foreign polypeptide is of interest if and only if it is therapeutic and/or otherwise useful in some manner, and if it has a desired and useful “neuroactive” property (as described below), and if delivery of the polypeptide into BBB-protected brain or spinal tissue is desired and intended. This requirement is intended to rule out and exclude, as prior art, viral infections or other non-useful infections, tests, or other procedures that involved attacks or challenges to brain or spinal tissue, via routes that included BBB-straddling neurons. It should be understood that numerous research tests have been done on lab animals, using wild-type viruses that can infect neurons or glial cells. As examples, such studies are often used in animals, to evaluate anti-viral drug candidates; they also are used as “tracer” studies to analyze neuronal networks and connections, such as by infecting animals with a pathogenic virus such as rabies, then sacrificing the animals after a predetermined number of hours, and then analyzing samples of brain tissue to see which neurons were infected by the spreading viruses, and in what sequence. Clearly, numerous tests of that nature have used viruses to introduce foreign polypeptides into BBB-protected brain or spinal tissue (and some of those tests used pathogenic viruses which had been genetically engineered to include epitopic tag sequences, discussed below, to make it easier to analyze for the presence and concentration of the viruses). However, just as clearly, inflicting those types of infections on research animals (most of which were killed, as part of the experiment) were not designed to benefit the animals that were being tested. Accordingly, this invention is substantially different from such “tracer” and similar tests, since this invention is designed to provide a method for introducing therapeutic or otherwise useful and beneficial polypeptides into BBB-protected tissue, in fields such as human medical therapy, and in breeding of livestock or endangered species.

The reference to “neuroactive” polypeptides is also a necessary part of the third factor discussed above. As used herein, “neuroactive” polypeptides includes those polypeptides which can exert a therapeutic or otherwise useful and/or desirable result or effect, if properly delivered to one or more desired and intended region(s) of brain or spinal tissue that is/are protected by the BBB. Various examples are discussed below and listed in various tables herein. It should be noted that “useful” and “neuroactive” polypeptides, as used herein, may include polypeptides that will exert direct effects only on glial cells, without requiring direct effects on neurons, if the treated glial cells generate a response that leads to a therapeutic or other beneficial result among neurons that are in fluid communication with the treated glial cells.

Also, terms such as “polypeptide” or “neuroactive polypeptide” include variants, derivatives, and fragments of naturally occurring or genetically engineered polypeptides. Examples include: (i) “chimeric” or “fusion” polypeptides, which have a neuroactive portion derived from one gene, and a “leader” sequence (to increase stability, transport, secretion, or some other useful trait or activity) from some other gene; and, (ii) fragments or portions of polypeptides (such as a single-chain binding fragment which has been isolated from the variable domain of a monoclonal antibody).

Since this invention relates to methods and compounds for transporting useful neuroactive polypeptides across the BBB, the following sections contain a brief overview of several examples of peptides that have very good potential for therapeutic activity inside CNS tissue.

### TYPES OF CNS-ACTIVE POLYPEPTIDES

A wide range of peptide neurotransmitters and neurohormones have been described which modulate nervous system function within the CNS (many of these peptides also have the same, similar, or occasionally different activities outside the CNS, in the “peripheral” nervous system). The following section offers a brief overview of just four of the more important categories of “CNS active polypeptides”.

Neurotrophic Factors

The root word “-troph” comes from the Greek work for “nourishment” or “food”. Accordingly, the term “trophic” implies that a certain molecule is involved in the nourishment, growth, stimulation, sustenance, or similar support of a certain system. Within the CNS, the term “neurotrophic factors” has come to refer to molecules which promote neuronal growth, create synaptic connections, or carry out other stimulating or supporting activities involving neurons; however, this term excludes both (i) essential nutrients (such as oxygen, glucose, amino acids, and nucleotides) which are required by all cells, and (ii) traditional neurotransmitters, which directly modulate nerve impulses between neurons (such as glutamate, acetylcholine, dopamine, serotonin, etc.).

Accordingly, most neurotrophic factors function in a manner similar to hormones; they are secreted by one type of cell, and subsequently interact with other cells. However, they differ from hormones in that, after secretion, they typically interact only with neighboring cells. In some respects, they act in a manner comparable to “paracrine” factors; this term describes a hormone-like molecule that acts only in a local manner, as contrasted with “endocrine” factors, which act in a systemic manner, including on cells distant from the secreting cell.

Neurotrophic factors are discussed in detail in review articles such as Lindsay 1994, Snider et al 1994, Bothwell et al 1995, Lewin et al 1996, and Skaper et al 1998, and in numerous US patents (including, for example, U.S. Pat. No. 5,169,762, Gray and Ullrich, 1992). The first neurotrophic factor that was discovered to stimulate nerve cell growth was called simply “nerve growth factor” (NGF). Subsequently, numerous other neurotrophic factors were discovered, and they required more elaborate names, such as brain-derived neurotrophic factor, ciliary neurotrophic factor, glial-cell-derived neurotrophic factor (GDNF), and names with numbers in series, such as neurotrophin-3, neurotrophin-4, etc.

A large number of neurotrophic factors are polypeptides, and these polypeptides offer outstanding potential as therapies for a wide variety of CNS disorders. Such therapies include treatments for brain damage caused by crisis, such as a traumatic injuries (such as automobile or other accidents, concussions, etc.), and ischemic or excitotoxic brain damage following a stroke, near-suffocation, cardiac arrest, or loss of blood. Neurotrophic polypeptides also offer great promise in treating, retarding, or preventing various neurodegenerative diseases, such as Alzheimer's disease, Parkinson's disease, Huntington's disease, amyotrophic lateral sclerosis, etc.

For convenience, terms such as disorder, damage, and injury are used herein to refer to any CNS disorder (whether from trauma, disease, etc.) which might be prevented, treated, or otherwise ameliorated by one or more therapeutic polypeptides, if such polypeptides can be delivered to the proper regions of the brain or spinal cord.

The therapeutic potential of neurotrophic polypeptides for treating CNS disorders is discussed and documented in a large number of articles, reviews, and books, including Hefti 1994, Thoenen et al 1994, Ibanez et al 1995, McMahon and Priestley 1995, and Yuen and Mobley 1996.

Hypothalamic Releasing Factors

The hypothalamic releasing factors are short polypeptides that are made and secreted by nerve cells in the hypothalamus. These polypeptides in turn stimulate the pituitary gland to secrete various hormones that control and affect numerous important body functions involved in growth, metabolism, water and salt balance of the blood, reproduction, etc. Like neurotrophic factors, hypothalamic releasing factors are potent, and are the subject of considerable study. Review articles that describe these molecules include Turnbull et al 1999, Phelps et al 1999, and Sawchenko et al 2000.

The medical and commercial use of hormones to control and regulate functions such as growth and reproduction is widespread. Growth hormone, for example, is now made commercially by recombinant methods, and used to treat certain types of retarded growth in children, and to accelerate growth in livestock. Given that the production and secretion of growth hormone and other important pituitary hormones is under the control of hypothalamic releasing factors released from the brain, delivery of hypothalamic releasing factors to the pituitary via delivery into the brain presents a novel alternative method of regulating body functions such as growth and reproduction.

Peptide Neurotransmitters

A range of peptide neurotransmitters have been described, including Substance P, enkephalins, endorphins, vasointestinal peptide (VIP), calcitonin gene related peptide (CGRP), galinin, somatostatin, etc. In many cases, peptide neurotransmitters act in a manner similar to classical neurotransmitters such as glutamate or acetylcholine; they are released by a neuron at a synapse, where a binding reaction to a specific receptor on the other neuron at the synapse stimulates or inhibits the propagation of a nerve signal (also called a nerve impulse, firing, or depolarization). In other cases, binding of a peptide neurotransmitter to a receptor at a synapse exerts a more prolonged effect, such as increasing or decreasing the sensitivity of the target neuron to other neurotransmitters.

Different neurotransmitter peptides often work to regulate the function of nervous circuits in the CNS. In the perception of pain, for example, Substance P is released by nocioceptive or pain-transmitting nerve fibers in the spinal cord, and is excitatory. Enkephalins and endorphins, on the other hand, inhibit transmission of pain signals. The potent pain relief of morphine is due to binding to endorphin receptors within the CNS; however, the use of endorphins for the treatment of pain is not possible at present, largely because of the difficulty in safely delivering such peptides into the brain.

Cytokines and Other Growth Factors

While some cytokines and other growth factors can function as neurotrophic factors by acting locally to stimulating nerve growth, in general, they can be distinguished from neurotrophic factors by their ability to also robustly stimulate growth and mitosis of various non-neuronal and dividing cells of the CNS. Examples of such polypeptides include acidic and basic fibroblast growth factor (aFGF and bFGF), epidermal growth factor (EGF), platelet derived growth factor (PDGF), insulin-like growth factor-I and II (IGF-I and IGF-II), tumor necrosis factor-B (TGF-B), leukemia inhibitory factor (LIF), various interleukins, etc.

These molecules are involved in normal physiological processes such as CNS tissue growth, remodelling and repair after injury, and immune responses within the CNS. While they have potential to be used to treat response to CNS injury or infection, they also pose a risk of stimulating emergence of cancerous cells, especially if administered in a systemic fashion throughout the CNS. Nevertheless, they are of great interest to neurology researchers, and development of methods delivering cytokines and similar molecules to discrete and limited CNS regions would be of enormous research and potentially therapeutic benefit.

Table 1 provides a partial listing of known CNS-active polypeptides. To illustrate just one area of application in slightly more detail, Table 2 provides examples of some CNS disorders, along with the polypeptides that are currently thought to have the greatest promise in treating or preventing these disorders.

It must be emphasized that Tables 1 and 2 contain only partial listings, for illustrative purposes. With the explosion of gene and protein sequence data that is emerging from the human and mouse genome sequencing projects, scientists are very actively identifying numerous other CNS-active polypeptides.

It must be kept in mind that the biggest and most difficult obstacle which is hindering both (i) careful and reliable evaluation of such potential therapies, and (ii) actual use of such therapies in patients who need such help, is the severe difficulty of actually delivering CNS-active polypeptides to the regions of the brain and spinal cord where those polypeptides will need to contact neurons or glial cells in order to provide a therapeutic or preventative benefit. Accordingly, the purpose and goal of this invention is to disclose methods of introducing foreign (exogenous) polypeptides into CNS tissue which is protected by the BBB.

### PRIOR ART CNS DRUG DELIVERY METHODS

Reviews of the drug delivery literature (e.g., Langer et al 1989; Langer 1990; Madrid et al 1991; Thoenen et al 1994; Zlokovic 1995, Cloughesy et al 1995, Davis et al 1995, Abbott et al 1996, Begley 996, Kroll et al 1998, Pardridge et al 1998, Rochat et al 1999, and Rapoport 2000) identify few currently available, effective, and clinically practical alternatives for delivering polypeptides across the blood brain barrier. A review of some of the approaches to solving the BBB drug delivery problem that have been tried, albeit without great success, may be found in articles such as Zlokovic 1995 and Rapoport 2000. These potential approaches can be categorized and briefly summarized as follows:

1. Invasive methods, which require surgical intrusion, or at a minimum, use of a hypodermic needle to puncture cells and membranes. One such method is “intraventricular” injection or infusion, which require a neurosurgeon to gain access to one of the “ventricles” that serve as production sites or gathering nodes for cerebrospinal fluid, so that a fluid can be injected into the ventricle. Except for the fourth ventricle (which is somewhat accessible, but which is at an exit location and therefore is not useful for administering drugs to the brain), these ventricles are buried deep inside the brain. Therefore, injecting drugs into brain ventricles, even if only with a needle, is difficult and dangerous. Any penetration by a cutting tip or cauterizing blade will inevitably damage capillaries along-the way, and if blood leaks out from damaged capillaries, it will contact neurons that need to be protected from blood by the BBB. If cautery is used to minimize blood leakage, then any capillaries and small arteries and veins that are cauterized will no longer carry blood, and neurons that were served by those blood vessels may be killed.

Another invasive method involves transplantation of genetically selected and/or engineered cells, which can secrete neurotrophic molecules at high rates, into target regions of the brain (e.g., U.S. Pat. No. 5,082,670, Hefti et al 1992). The insertion of such cells into a target portion of the brain requires the same types of invasive access (using blades or needles) that pose serious problems and dangers in intraventricular injection. Because of these problems, these types of invasive techniques are effectively limited to animal tests intended to evaluate the effects of such molecules, and are not used in humans except in limited clinical trials, where a patient is suffering from a terminal illness, or a debilitating disease such as Parkinson's disease that has reached a level of severity which renders the patient unable to be significantly helped by any other treatment.

2. Non-invasive methods, which use chemical, cellular, carrier-mediated, or other non-surgical methods to transport neurotrophic or similar molecules across the blood-brain barrier. These methods typically require fairly large quantities of vesicles, complexes, or other macromolecules to be injected into the bloodstream, in the hope that some quantity of the injected “passenger” molecules will enter the brain and be transported across the BBB, into brain tissue. Examples of these approaches are described in items such as U.S. Pat. No. 5,154,924 (Friden 1992), a chapter by Thoenen et al in Racagni et al 1994, and Zlokovic 1995.

One of the primary problems with non-invasive approaches that attempt to use carrier molecules and methods to transport neuroactive compounds into the brain is that, if a compound can indeed manage to permeate the tight capillary junctions that form the blood-brain barrier in any significant quantity, that same compound will much more easily and readily pass through the much larger “slit-pores” that render capillary walls much more permeable, throughout the remainder of the body. This leads directly to two main categories of potentially serious problems. The first class of problems relates to side effects that can be provoked by pumping large quantities of neuroactive compounds into non-CNS tissue; such compounds may disrupt or affect the peripheral nervous system, or other types of cells or tissue. The second class of problems relate to the very high expenses that are imposed by the requirements of careful synthesis, purification, and quality testing of compounds that must be injected or infused in large quantity. Such injections or infusions usually must be repeated, often numerous times; and, after an injection or infusion of a large quantity of a neuroactive compound, a patient must be monitored for at least a day or more, usually in a hospital or well-staffed clinic setting rather than at home, so that emergency measures may be taken immediately if the patient suffers an adverse reaction.

In addition, the prior art methods do not allow foreign polypeptides to be administered only to selected neurons, classes of neurons, or brain regions. By contrast, this invention offers a number of promising approaches to administering foreign polypeptides to targeted neurons in a controllable and selective (or at least enriched) manner. By targeting only certain locations or regions inside the CNS, and/or by targeting only certain types or clusters of neurons, the risk and severity of side effects can be minimized, and the benefits of treatments using very small quantities of foreign polypeptides can be increased.

### GENE THERAPY

One approach which may have potential for use in treating CNS disorders, such as Alzheimer's disease or Parkinson's disease, involves gene therapy. In prior art efforts to genetically modify neurons that are protected by the BBB, two general categories of approaches have been used. In one type of approach, cells are genetically altered, outside the body, and then transplanted somewhere in the CNS, usually in an area inside the BBB.

In the other type of approach, genetic “vectors” are injected directly into one or more regions in the CNS, to genetically alter cells that are normally protected by the BBB. Because serious obstacles arise when attempts are made to adapt procedures that work quite well, in other parts of the body, to the special challenges that arise when BBB-protected brain or spinal tissue is involved, these two types of approaches are described in a bit more detail below, under separate subheadings.

Transplant of Genetically Engineered Cells

In conventional tissue, this approach involves isolating a type or population of cells, manipulating those cells outside the body (“ex vivo” and “in vitro” are terms often used to describe this type of processing, using cell culture media), and then transplanting the manipulated cells into the animal or patient. In some cases, the cells to be manipulated are obtained from the host, to avoid or minimize potential immune rejection problems after implantation into the host; in other cases, they may be from some other source (such as from an immortal cell line, from fetal or stem cells, etc.). The process of genetic manipulation may involve specific and precisely-controlled techniques, or it may involve “shotgun” approaches, where a wide range of outcomes can arise among a treated population of cells, and the treated cells are screened, in an effort to identify one or more cell lines that happened to receive and stably integrate a desired mutation.

Extension of this general approach and application to the CNS is not practical, for a number of reasons, including the following brief listing. First: the CNS is heterogeneous in the extreme, and nervous tissue from one region can not be substituted for nervous tissue from another region. Second: CNS neurons in adults are post-mitotic, and will not divide and repopulate if a vacancy is created. Third: nerve cell processes or axons, once broken, do not readily regrow and reestablish synaptic and other connections. Fourth: surgical or similar extraction of CNS tissue poses great risks to a patient, even if the surgery is done with the highest level of skill and care.

For these and other reasons, it simply is not feasible or practical, in the vast majority of cases, to attempt to remove a population of CNS neurons from a patient, genetically manipulate the neurons, and then return them back into the patients's brain or spinal cord.

Accordingly, most experiments in the field of implanting genetically engineered cells into CNS tissue have not even attempted to use cells that were taken from the same patient. Instead, early tests used genetically engineered cells (not necessarily neurons) that had been modified to secrete abnormally large quantities of some desired protein (such as nerve growth factor, for transplantation into the brains of patients suffering from Alzheimer's disease).

While some results have encouraged further research, most research in this field is exploring the possibility of transplanting other types of cells (such as stem cells or fetal cells) into CNS tissue. These types of tests and treatment efforts are described in articles such as Blesch and Tuszynski 1996, Karpati et al 1996, Fick and Israel 1994, Fisher and Ray 1994, and Friedman 1994, and in patents such as U.S. Pat. No. 5,082,670 (Hefti et al 1992). Although this line of research is quite interesting and potentially very useful and valuable, the bottom line is that, in order to successfully correct any serious disorder of the brain or spinal cord, any genetically modified stem or fetal cells will need to be implanted inside CNS tissue, in a way that must puncture or cut through the BBB. Such methods are necessarily invasive, and inevitably injurious to the CNS.

By contrast, genetic manipulation of CNS cells in situ offers substantially better hope and promise for truly correcting a problem without inflicting invasive damage on the patient's brain and/or spinal cord.

Genetic Alteration of CNS Cells In Situ

This approach aims to introduce into the CNS a source of a desirable polypeptide, by genetically engineering cells within the CNS. In the prior art, this has been achieved, a number of times, by directly injecting a genetic vector into the CNS, to introduce foreign genes into CNS neurons “in situ” (i.e., neurons which remain in their normal position, inside a patient's brain or spinal cord, throughout the entire genetic transfection or transformation procedure).

The primary problem with the prior art approach of injecting genetic vectors directly into CNS tissue that is protected by the BBB is that it is invasive. The BBB must be punctured or cut, somehow, by a needle or blade. What is needed, instead, is a non-invasive method of genetically transfecting or transforming cells that sit inside the BBB, protected by the BBB, without ever puncturing, cutting, or damaging the BBB.

It should be noted that the terms “transfect” and “transform” are used interchangeably herein. Both terms refer to a process which introduces a foreign gene (also called an “exogenous” gene) into one or more preexisting cells, in a manner which causes the foreign gene(s) to be expressed to form corresponding polypeptides. As used by some scientists, “transfect” implies that any foreign gene(s) are likely to be expressed only transiently in the cells, in a manner analogous to an infection which lasts only for a while and is eventually stopped. By contrast, “transform” implies a permanent genetic alteration that will be passed on to any and all progeny cells, usually due to integration of the foreign gene(s) into one or more cell chromosomes. However, the boundary lines between those terms can become blurred in various situations, and the distinctions between those terms are not always applied consistently by all scientists. Accordingly, “transfect” and “transform” are used interchangeably herein, regardless of how long a foreign gene might continue to be expressed after it enters target cell(s).

### GENE VECTORS

There are two broad classes of genetic- vectors. The viral vectors are derived from viruses, and make use of the lipid envelope or surface shell (also known as the capsid) of a virus. These vectors emulate and use a virus's natural ability to (i) bind to one or more particular surface proteins on certain types of cells, and then (ii) inject the virus's DNA or RNA into the cell. In this manner, viral vectors can deliver and transport a genetically engineered strand of DNA or RNA through the outer membranes of target cells, and into the cells'cytoplasm.

Construction of a viral vector usually requires at least two major steps. One step involves removing or disabling at least one (and frequently more than one) selected gene within the virus chromosome, such as one or two genes that drive replication of the virus. Without these gene in its genome, the crippled virus cannot make more virus particles, and cannot reproduce (replicate) itself, inside normal cells. However, in a laboratory, any desired quantity of the virus can be made, by growing the crippled virus inside host cells that have been genetically engineered to express the genes deleted from the virus. Accordingly, the crippled viruses can readily reproduce, but only inside special host cells that do not exist outside a laboratory.

The second major step in making a viral vector involves substituting or inserting one or more desirable genes (often called “passenger” genes), which encode a desirable polypeptide, into the viral genome.

In this manner, the viral vector becomes a delivery system, which can inject a modified viral genome that carries the passenger gene(s) into susceptible cells. After the viral DNA enters the cell, the desirable gene is expressed into a desirable polypeptide by the normal cellular mechanisms. This will make multiple copies of the desired polypeptide, but because the vector lacks the genes needed to replicate, a pathogenic infection is (at least in theory) not a substantial risk.

Gene transfers into CNS neurons have been reported using such vectors derived from herpes simplex viruses (e.g., European Patent 453242, Breakfield et al 1996), adenoviruses (La Salle et al 1993), and adeno-associated viruses (Kaplitt et al 1997).

The second broad class of genetic vectors are termed non-viral vectors. These vectors typically contain a “gene expression construct”. This phrase is understood to those skilled in the art; by way of illustration, such a construct might be a plasmid, which normally would contain (i) a bacterial origin of replication, so that it can be grown quickly and cheaply under laboratory conditions, inside suitable bacterial cells such as E. coli; (ii) at least one marker gene, such as a selectable marker gene that will allow cells containing the plasmid to survive on an unusual nutrient source or in the presence of a toxic antibiotic, or a scorable marker gene that will cause cells containing the gene to turn a certain color or exert a fluorescent or other trait under certain conditions; and, (iii) at least one passenger gene, containing a suitable promoter and a coding sequence which will express a desired polypeptide, once the gene expression construct is inside transfected mammalian cells.

The non-viral genetic vector is then created by adding, to the gene expression construct, selected agents that can aid entry of the gene construct into target cells. Several commonly-used agents include cationic lipids, positively charged molecules such as polylysine or polyethylenimine, and/or ligands that bind to receptors expressed on the surface of the target cell. For the purpose of this discussion, the DNA-adenovirus conjugates described by Curiel (1997) are regarded as non-viral vectors, because the adenovirus capsid protein is added to the gene expression construct to aid the efficient entry of the gene expression construct into the target cell.

An overview of several major known categories of non-viral gene vectors follows.

1. Cationic Gene Vectors

DNA strands are negatively charged, and cell surfaces are also negatively charged. Therefore, a positively-charged agent can help draw them together, and facilitate the entry of the DNA into a target cell. Examples of positively-charged transfection agents include polylysine, polyethylenimine (PEI), and various cationic lipids. The basic procedures for preparing genetic vectors using cationic agents are similar. A solution of the cationic agent (polylysine, PEI, or a cationic lipid preparation) is added to an aqueous solution containing DNA (negatively charged) in an appropriate ratio. The positive and negatively charged components will attract each other, associate, condense, and form molecular complexes. If prepared in the appropriate ratio, the resulting complexes will have some positive charge, which will aid attachment and entry into the negatively charged surface of the target cell.

The use of liposomes to deliver foreign genes into sensory neurons is described in various articles such as Sahenk et al 1993. The use of PEI, polylysine, and other cationic agents is described in articles such as Li et al 2000 and Nabel et al 1997.

2. Receptor-Targeting Gene Vectors

An alternative strategy for introducing DNA into target cells is to associate the DNA with a molecule that normally enters the cell. This approach was demonstrated in liver cells in U.S. Pat. No. 5,166,320 (Wu et al 1992). An advantage of this approach is that DNA delivery can be targeted to a particular type of cell, by associating the DNA with a molecule that is selectively taken up by that type of target cell.

This type of “selective uptake” depends on cell receptors, which typically are proteins that straddle the outer membrane of a cell, with part of the receptor protein exposed outside the cell, and part of the protein anchored inside the cell cytoplasm. In order to be suitable for this type of receptor-mediated uptake, the cell receptor should be involved in a process called “endocytosis”, which means that, once a suitable ligand binds to the receptor, the reaction that follows will cause both the receptor, and the ligand that is bound to it, to be pulled into the cell. This usually involves a cell membrane process called “invagination”, and is discussed in any number of texts on cell biology.

If a “passenger” molecule is coupled to a “receptor targeting” ligand that can serve as a “carrier” molecule, receptor-mediated endocytosis is likely to cause the cell to swallow up the entire complex (the receptor, the carrier molecule which has bound to the receptor as a ligand, and the passenger molecule which is bound to the carrier molecule). Most carrier-passenger complexes usually are created by using ionic attraction, rather than covalent bonding, to make it easier for the passenger molecule to separate from the carrier molecule, after the complex enters a cell.

A limited number of molecules are known to undergo receptor mediated endocytosis in neurons. Known agents that bind to neuronal receptors and trigger endocytosis, causing them to enter the neurons, include (i) the non-toxic fragment C of tetanus toxin (e.g., Knight et al 1999); (ii) various lectins derived from plants, such as barley lectin (Horowitz et al 1999) and wheat germ agglutinin lectin (Yoshihara et al 1999); and, (iii) certain neurotrophic factors (e.g., Barde et al 1991). At least some of these endocytotic agents undergo “retrograde” axonal transport within neuron. The term “retrograde”, in this context, means that these molecules are actively transported, by cellular processes, from the extremities (or “terminals”) of a neuron, along an axon or dendrite, toward and into the main body of the cell, where the nucleus is located. This direction of movement is called “retrograde”, because it runs in the opposite direction of the normal outward (“anterograde”) movement of most metabolites inside the cell (including proteins synthesized in the cell body, neurotransmitters synthesized by those proteins, etc.).

The fact that certain molecules undergo receptor mediated retrograde transport in certain neurons suggests that they may be useful for targeting delivery of genes to particular neurons expressing receptors for those molecules. The prior art contains very few examples of this. Very recently, Knight et al 1999 documented a non-viral vector that uses the C fragment of the tetanus toxin as a “carrier” molecule for DNA.

The development of novel non-viral vectors that make use of the receptor targeting approach is likely to be accelerated by availability of a much wider range of ligands that have been shown to undergo receptor-mediated endocytosis and retrograde transport in neurons. A process for identifying, isolating, and generating such ligands, using phage display libraries which can be reacted with neuronal tissue in numerous repeated cycles to identify and select the most promising ligands, is disclosed herein.

3. Other Non-Viral Genetic Vectors

Cationic gene vectors and receptor-targeting genetic vectors are not as efficient as viral vectors in leading to expression of foreign genes by target cells. This is primarily due to the mechanism of uptake of DNA complexes by target cells, during endocytosis. When a target cell internalizes a receptor complex formed by the receptor and the ligand which is coupled to it (and also including a DNA segment, if the ligand was used as a carrier molecule to help internalize the DNA segment), the invagination process leads to the DNA complex being surrounded by a tiny lipid sphere. That tiny sphere is called an endocytotic vesicle.

Before the DNA can begin acting as a gene (i.e., before the foreign gene sequence can be transcribed into mRNA, which is then translated into a polypeptide by the protein synthesis machinery of the target cell), the DNA must first escape from the endocytotic vesicle.

While this may occur without great difficulty with viral vectors, escape from the endocytotic vesicle can be relatively inefficient with many non-viral genetic vectors, and that factor can be an obstacle to efficient transfection.

However, many viruses (including adenoviruses) have evolved capsid molecules that enable efficient fusion or rupture of endocytotic vesicles, thereby enabling efficient release of the DNA inside the cell cytoplasm. Therefore, attaching an adenoviral capsid protein of this type to a gene expression construct, to form a DNA-adenovirus conjugate, can result in improved and more highly efficient gene transfection, as described by Curiel (1997). Similarly, the transfection efficiency of a cationic gene vector or receptor-targeting gene vector can be enhanced, in some cases, by utilizing an adenoviral capsid or capsid protein (possibly as one portion of a fusion protein) which can carry out that function.

### VIRUS INFECTIONS OF THE CNS

While some types of viruses can be engineered to efficiently deliver desirable genes, it has always been clear to these researchers that any such viruses must be genetically engineered to prevent their replication. If a viral vector can introduce its genes into a neuron and then replicate in that neuron, then the infection can spread from neuron to neuron and often into the CNS resulting in viral encephalitis.

The fact that viruses can spread from neuron to neuron has allowed researchers to use the spread of virus to identify which neurons are connected into systems or pathways. This process is call trans-neuronal tracing. and is an established tool used in the area of neuroscience research concerned with study of the anatomical relationships within the brain and spinal cord.

It previously has gone unreported that some of the neurons that become infected by viruses in such transneuronal tracing studies show the same or similar patterns of neurodegeneration that occur in a number of important CNS disorders. As one example, there are substantial similarities between the patterns of upper motor neuron degeneration that are seen in patients suffering from amyotrophic lateral sclerosis, and patterns of upper motor neuron infection and damage that are caused by poliovirus infections. As another example, the degeneration of basal forebrain cholinergic neurons that is an early and invariable feature of Alzheimer's disease shows important similarities to damage to a neuronal population known as the horizontal limb of the diagonal band of Broca (HDB), which occurs following administration of the rabies virus to the olfactory epithelium in the nose. These previously undocumented observations are disclosed herein, because the similarities between patterns of viral infection in CNS tissue, and patterns of neurodegeneration in various CNS disorders, suggests that the methods disclosed herein (i.e., delivering therapeutic polypeptides non-invasively into the brain or spinal cord, in a manner which relies on certain neurons to transport and deliver polypeptides to other neurons that share contact points with the carrier neurons) offers the promise of both: (i) potentially powerful therapeutic treatments, and (ii) potentially powerful experimental approaches that can be used by researchers to, for example, selectively alter patterns of gene expression and/or nerve cell function within the brain or spinal cord, and/or to elucidate various networks and principles within CNS tissue, including factors and principles that govern the response of CNS tissue to assaults ranging from viral infections to neurodegenerative diseases.

Despite all of the research in this field of neuroscience, and all of the extraordinary advances in molecular biology in recent years, there still remains a major and unmet need for improved methods of transporting useful polypeptides across the blood-brain barrier, so that they can provide therapeutic benefits to neurons in brains and spinal cords, without requiring invasive damage to the brain using surgery, or needles that must penetrate the skull and brain tissue.

Accordingly, one object of this invention is to disclose a non-invasive method of transporting polypeptides across the blood-brain barrier, using transfection of sensory neurons (such as olfactory neurons) which “straddle” the blood-brain barrier, in a manner which causes the BBB-straddling neurons to deliver therapeutic polypeptides to neurons which are located entirely within the blood-brain barrier.

Another object of this invention is to disclose a non-invasive method of transporting polypeptides across the blood-brain barrier, using motor neurons (or, if desired, neurons of the autonomic nervous system) which “straddle” the blood-brain barrier to deliver therapeutic polypeptides to neurons located entirely within the blood-brain barrier.

Another object of this invention is to use genetic engineering methods and compounds to provide a method of transporting polypeptides into CNS tissue, in a manner which can be used to selectively affect targeted clusters, regions, or types of CNS neurons located entirely within the blood-brain barrier.

These and other objects of the invention will become more apparent through the following summary, drawings, and description of the preferred embodiments.

### SUMMARY OF THE INVENTION

The present invention describes a method for the noninvasive transport and delivery of polypeptides through the blood brain barrier (BBB) and into the central nervous system (CNS) of a mammal or other higher animal, allowing the polypeptides to contact cells located wholly within the BBB. This method enables previously unavailable forms of medical or veterinary treatment, in humans and other animals, and can also be used for commercial purposes, in livestock. The method is accomplished by using a genetic vector to transfect a selected type of neuron which “straddles” the blood brain barrier; examples include sensory neurons such as olfactory or nocioceptive neurons, motor neurons, and pre-ganglionic neurons of the autonomic nervous system. The genetic vector is introduced and administered to the patient or animal in a manner which causes it to contact and transfect a neuronal “projection” which extends outside the BBB. Once inside a transfected “primary” neuron which straddles the BBB, a vector-borne gene which encodes a CNS-active polypeptide will be transported from the neuronal projection into the main cell body, where it will be expressed by the BBB-straddling neuron to form therapeutic or otherwise useful polypeptide molecules. These polypeptides will be transported within the transfected neuron to one or more secretion locations that are positioned within CNS tissue that is protected by the BBB. The polypeptides will be secreted by the transfected primary neuron at that location inside the BBB, and will then be able to contact and deliver to “secondary” target neurons which are located wholly within (and are therefore protected by) the blood-brain barrier. Using this system, polypeptides such as neurotrophic factors can be noninvasively delivered to, and used to treat, target neurons which lie wholly within the BBB. This allows new methods of treating CNS neurons that have been injured, or which degenerate in neurodegenerative diseases. Alternately, this new method allows polypeptides that can suppress neuronal activity (such as neurotrophic factor antagonists) to be transported across the BBB in a manner which can help control and reduce problems involving an unwanted excess of neuronal activity, such as neuropathic pain. This approach also allows new methods of delivering polypeptides that can modulate endocrine functions into the CNS, thereby allowing improved treatment of various medical problems among humans, and improved ability to modulate growth, maturation, reproduction, or other endocrine-related functions among livestock, endangered species, and other animals.

### BRIEF DESCRIPTION OF THE DRAWINGS

FIG. 1 depicts the use of a genetic vector to transfect an exposed projection (“mucosal tip”) of an olfactory receptor neuron, for the purpose of delivering therapeutic polypeptides through the BBB to neurons that reside wholly within the BBB. The transfected olfactory receptor neuron will express the vector-borne gene into polypeptides, which will then be transported through a neuronal axon which crosses the BBB. The polypeptides will then be secreted inside the BBB, by the BBB-straddling neuron, where they can contact CNS neurons that are located wholly inside the BBB, such as cholinergic neurons in the basal forebrain.

FIG. 2 depicts a viral vector which can transfect neurons; this vector includes a capsid shell, binding ligands on the surface, and a genetically-engineered genome which contains a useful “passenger” or “payload” gene.

FIG. 3 depicts transfection of an olfactory receptor neuron, and depicts the route, through the olfactory bulb, that the vector-encoded polypeptide will travel to reach and contact a terminus, located at the tip of a neuronal process which is part of a cholinergic neuron that has its main cell body inside the basal forebrain.

FIG. 4 (which includes FIGS. 4A, 4B, and 4C) depicts the use of a genetic vector for the purpose of delivering anti-neurotrophic polypeptides to neurotrophic-factor-producing cells that reside wholly within the BBB, to reduce an unwanted excess of neuronal connections or activity, as occurs in conditions such as neuropathic pain. In this method, an “NPC” (neuropathic pain control) vector is used to transfect nocioceptive neurons which innervate tissues such as skin. The nocioceptive neurons will express vector-encoded polypeptides having anti-neurotrophic activity, and will release those polypeptides into spinal tissue. As depicted in FIGS. 4B (which shows abnormally large numbers of unwanted pain-signalling neuronal connections, before the treatment) and 4C (which shows a reduced number of unwanted pain-signalling connections, after the treatment), this type of blockade or inhibition of neurotrophic receptors or factors will lead to suppression or atrophy of the excessive neuronal connections that were causing or aggravating the neuropathic pain condition.

FIG. 5 (which includes FIGS. 5A, 5B, and 5C) depicts the use of a genetic vector to transfect “spinal motor neurons” in a muscle for the purpose of delivering therapeutic polypeptides to a class of neurons (called “upper motor neurons”) that lie wholly within the BBB. This type of treatment would be carried out, for example, in a patient with a limb that has become paralyzed or impaired due to stroke, injury, or disease. As shown in FIG. 5A, the gene vector will be injected into muscle tissue, where it will transfect spinal motor neurons that straddle the BBB; those neurons will express vector-encoded polypeptides having neurotrophic activity, and will release those polypeptides into spinal tissue, thereby delivering neurotrophic polypeptides to the upper motor neurons that are fully inside the BBB. FIG. 5B depicts a condition in a stroke-impaired or similar patient before treatment, in which some (but not all) of the neuronal “processes” which belong to upper motor neurons have degenerated and/or atrophied, and are no longer able to interact adequately with spinal motor neurons, leading to impairment or paralysis of a limb. FIG. 5C depicts an improved condition after treatment with a neuronal growth factor; in this condition, the still-functioning neuronal process which belongs to one of the upper motor neurons has created (“sprouted”) new and additional synaptic connections, which can interact with the spinal motor neurons that were not being adequately innervated prior to the treatment.

FIG. 6 depicts the use of a genetic vector to transfect projections of lower motor neurons of the hypoglossal nucleus (a part of the brain stem), for the purpose of delivering therapeutic polypeptides through the BBB to neurons that reside wholly within the BBB. The exposed ends of these neuronal projections are present in the tongue; therefore, this class of neurons offers a relatively direct route for introducing foreign polypeptides into the brainstem portion of the CNS.

FIG. 7 depicts the cyclic use of a phage library to select ligands that will bind to endocytotic receptors on BBB-straddling neurons.

### DETAILED DESCRIPTION

As suggested by the Summary section, above, this invention can be regarded has having four distinct elements (or factors, components, or similar terms). All four of these elements must work together, in a coordinated and sequential manner, in order to carry out the invention successfully. For purposes of analysis and explanation, those four elements can be divided and numbered, in an abbreviated summary, as follows:

A (1) suitable genetic vector carries a DNA sequence which includes at least one desired “passenger gene”, which may include a marker gene (to simplify detection and analysis, during research) and/or a payload gene (which encodes a therapeutic or otherwise desired and useful polypeptide). This type of vector is used to genetically transfect (2) one or more selected types of neurons that straddle the blood-brain barrier. After the passenger gene has entered a BBB-straddling neuron, the neuron will transport the gene to the main body of the neuron, where the gene will be expressed, thereby forming (3) “foreign” or “exogenous” polypeptide molecules, inside the transfected neuron. These foreign polypeptide molecules (which, in some cases, may be identical to “endogenous” or “native” polypeptides that are not being made in sufficient quantities by a patient) will then be transported, still within the transfected BBB-straddling neuron, to a secretion site, located inside the BBB. The polypeptide molecules will then be secreted by the transfected BBB-straddling neuron, at the secretion site inside the BBB. This type of secretion process will cause the foreign polypeptide molecules to contact (and to treat, modulate, or otherwise affect, in a therapeutic or other useful manner) other types of (4) “target” CNS neurons or other cells that lie entirely within the BBB.

In the discussion immediately below, a virus-derived vector is used to illustrate this method of treatment; however, other types of non-viral vectors alternately may be used, as discussed in later sections.

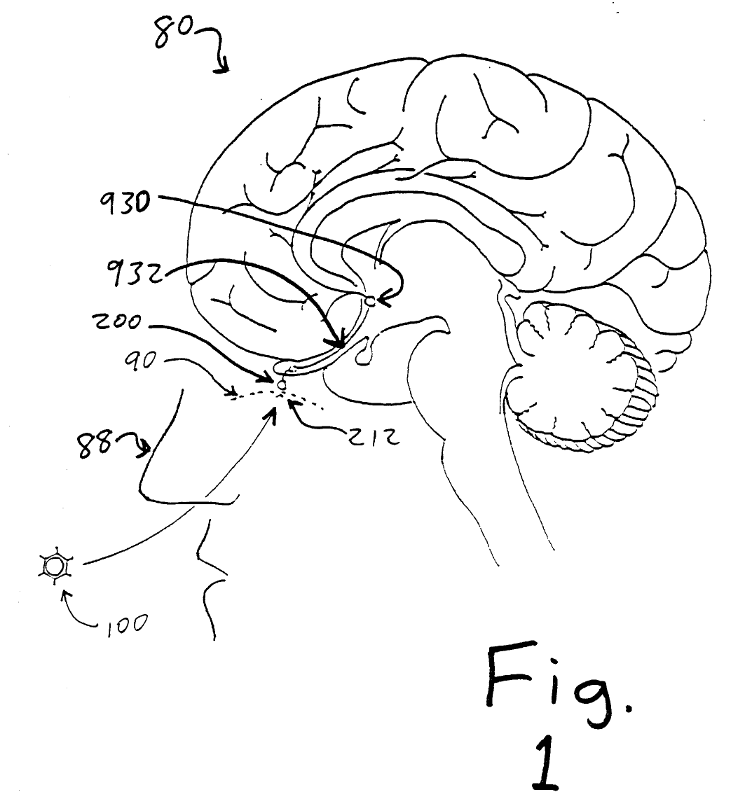

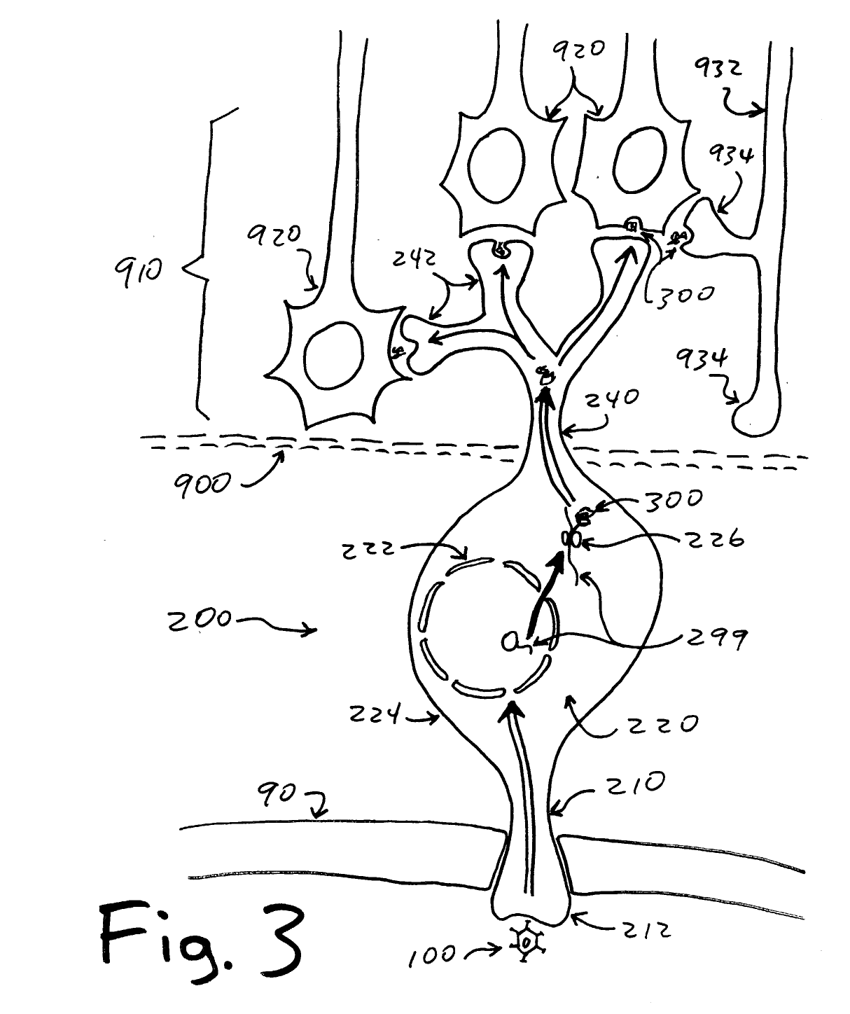

A first example which illustrates this method of treatment is illustrated in FIGS. 1 through 3, using consistent callout numbers in all three drawings. FIG. 1 depicts this treatment on a “macroscopic” level; a liquid containing numerous copies of genetic vector 100 is introduced into the nasal sinuses of patient 80. The vector contacts the exposed “peripheral projection” 212 (see FIG. 3) of olfactory receptor neuron 200; these neuronal projections are accessible on the surfaces inside the nasal sinuses, and they can be directly contacted by various airborne compounds as one of the biochemical processes involved in the sensation of smell.

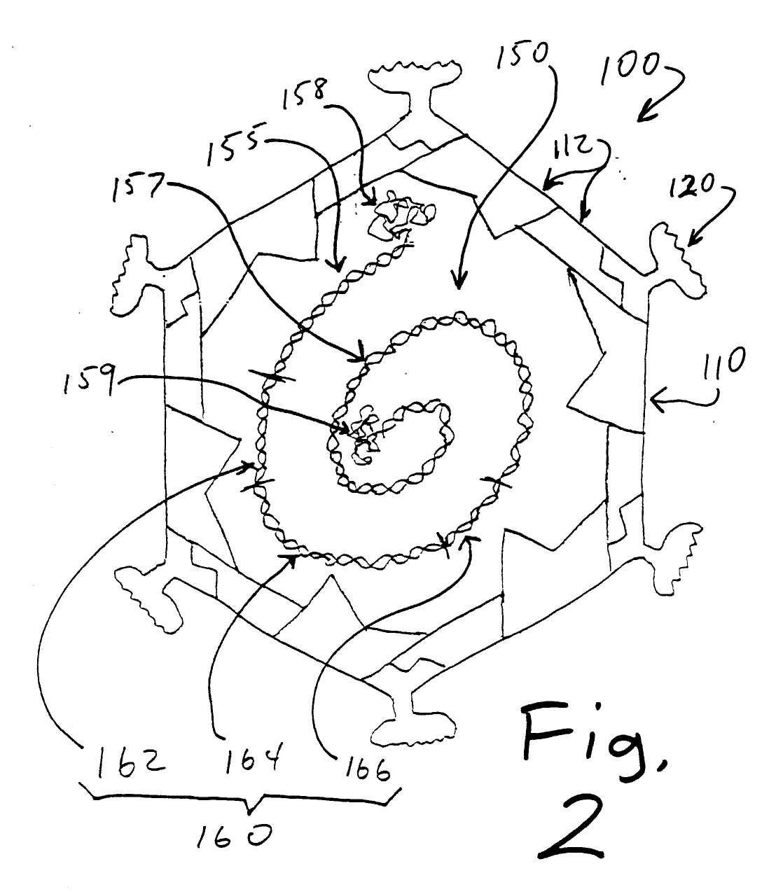

As shown on a different scale (at a microscopic, cellular level) in FIG. 3, once the DNA carried by the genetic vector 100 has entered a neuronal projection 212, it is then carried to the main cell body 224, by a process called “retrograde transport” (discussed below). Because the genetic vector in this example has been derived from a type of virus that is fully capable of infecting nasal receptor cells, the vector DNA (which carries passenger gene 160, as shown in FIG. 2, as merely one component inserted into an disabled but infective virus genome) is able to help promote and facilitate this process. Accordingly, the use of infective viruses to create the vector delivery system will help ensure that the transcribed portion of the passenger gene 160 will be expressed into messenger RNA strands 299 in at least some transfected neurons, as shown in FIG. 3.

The mRNA strands which have been transcribed from the passenger gene 160 will then be translated into multiple copies of foreign polypeptide molecules 300, by the normal cellular mechanism involving ribosomes 226 inside the transfected neuron 200.

Due to natural transport and secretion mechanisms (which can be enabled or enhanced, if desired, by using genetic engineering techniques as discussed below to add one or more specialized transport or secretion sequences to an end of a polypeptide chain), at least some of the foreign polypeptide molecules 300 will then be transported (using a process called anterograde transport) to various synaptic terminals 242 (or other peptide-secreting locations) that belong to the BBB-straddling neuron 200.

At these locations, which are located entirely within BBB-protected CNS tissue, the foreign polypeptide molecules 300 will be released by the BBB-straddling neuron 200. This secretion process allows the secreted foreign polypeptide molecules 300 to directly contact (and to begin exerting therapeutic or other modulating effects on) other classes of neurons which are referred to herein as “target” neurons; these “target” neurons lie wholly within the BBB. In the schematic illustrations shown in FIGS. 1 and 3, the polypeptides are secreted by the transfected BBB-straddling neuron 200 within a roughly spherical globular interface structure called a “glomerulus” 910, which contains various types of “targeted” neuronal structures, as discussed below.

To condense this process into short form, this overview and FIGS. 1-3 indicate how (1) a genetic vector carrying a passenger gene, is used to transfect (2) BBB-straddling neurons, which will then express (3) foreign polypeptides, which will then be secreted into CNS tissue inside the BBB, where they will contact and affect (4) “target” CNS neurons located wholly within the BBB.

Each of these four elements are described in more detail below, making use of the olfactory receptor neuron for illustration purposes. It will be apparent to those skilled in the art how to adapt and apply this description to other types of BBB-straddling neurons.

Since the optimal design of a suitable genetic vector will depend on the particular type of BBB-straddling neuron that is being transfected, the BBB-straddling neurons that will be transfected are described first.

### TYPES OF BBB-STRADDLING NEURONS

Neurons that straddle the BBB can be divided into three main classes: sensory neurons, motor neurons, and pre-ganglionic autonomic neurons. All of these classes of BBB-straddling neurons discussed have been studied extensively, and genetic vectors are known that can be used to transfect each of these classes of neurons.

The first major class of BBB-straddling neurons addressed herein are the sensory neurons, which are the “front line” cells that are directly involved in sight, smell, taste, and various sensations involved in touch, pain, the sense of position of a limb, etc.

Sensory neurons have specialized components (referred to herein as “peripheral projections”) that extend out to (or very near to) certain surface areas located outside the BBB. A “peripheral projection” is the portion of a BBB-straddling neuron which is the part of the cell (excluding the main cell body) that is most directly accessible to a genetic vector that will not need to cross the BBB in order to contact a surface of the projection.

Conversely, the term “central projection” refers to a fibrous portion of a neuron which extends away from the main cell body, and which extends either: (i) through the BBB, if the main cell body is located outside the BBB, as occurs with many types of sensory neurons; or, (ii) closer to the center or the brain or the upper spinal cord, if the main cell body is located inside the BBB, as occurs with motor neurons and pre-ganglionic autonomic neurons.

The term “axon” also should be noted and understood, with regard to central or peripheral projections. In most cases, the term “axon” refers to the single largest and longest projection that emerges from a neuron's main body. However, many sensory neurons can be regarded as having two axons, with one extending toward the periphery, while the other extends toward the brain and crosses the BBB. Because one of the primary roles of most BBB-straddling neurons is to shuttle nerve impulses between the peripheral and central nervous systems, the portion of a BBB-straddling neuron which actually crosses the BBB can almost always be properly classified as an axon.

Two main subclasses of sensory neurons are of primary interest herein, since they are especially useful for this invention. Those two subclasses are: (i) olfactory receptor neurons, which have projections that are accessible on the surfaces inside the nasal sinus; and, (ii) neurons of the dorsal root ganglia, such as various types of nocioceptive (pain-signalling) neurons which are distributed around the trunk, limbs, and extremities, in shallow layers beneath the skin. These will be discussed separately.

### OLFACTORY RECEPTOR NEURONS

One particular class of BBB-straddling neurons preferred for use as disclosed herein includes olfactory receptor neurons. Because of the role they play in the sense of smell, their peripheral projections are exposed and accessible on the interior surfaces of the nasal sinuses, where they can be directly contacted and activated by various types of airborne molecules that are being drawn in through the nose.

These neurons are described in some detail in various textbooks on physiology, such as Guyton and Hall, Textbook of Medical Physiology, 9th edition (1996), pages 678-681, and additional references cited therein on pages 681-682. Very briefly, the olfactory membrane in a human has a surface area of about 2.5 square centimeters, and typically has about 100 million receptor neurons. The exposed tip of each olfactory receptor neuron usually has about 6 to 12 tiny hairs called cilia, which extend downward several microns into a layer of mucus. It is presumed that most molecules which trigger perceptions of odor interact with the cilia and/or receptor proteins that straddle the membrane of the exposed surface of a receptor neuron. Although most molecules which trigger sensations of smell do not enter the neurons themselves, it is known that a few specific types of molecules (such as wheat germ agglutinin, which binds in a non-specific manner to most glycoproteins) are taken up and transported into olfactory receptor neurons, presumably by some form of endocytosis. Several published reports have also stated that adenovirus vectors, when used to contact the olfactory membrane, can indeed transfect (i.e., can insert foreign genetic material into) olfactory receptor-neurons, as evidenced by subsequent expression of marker genes by the transfected receptor neurons.

Olfactory receptor neuron 200, illustrated in a schematic manner in FIG. 3, comprises a number of cell parts that will be mentioned because of how they interact with one or more parts of the genetic vector 100. While the nasal sinus membrane 90 is shown schematically as a distinct layer, it is made up of the exposed apical surfaces of the olfactory support cells of the olfactory epithelium. A neuronal projection 210 passes through the nasal sinus membrane 90, creating an exposed terminus 212 (also called a mucosal tip, end, or knob). This exposed neuronal tip 212, which sits inside the nasal sinus cavity, allows the neuronal tip 212 to be contacted by genetic vectors (as disclosed herein) that are carried by a liquid nasal spray.

Olfactory receptor neuron 200 also comprises cell cytoplasm 220, a nucleus 222 which sits inside the main cell body 224, ribosomes 226 which translate mRNA into polypeptides, and an axon 240 which passes through the blood-brain barrier (BBB). As noted in the Background section, the BBB is not a single barrier-like structure; instead, it is a network of capillary walls that have unusually tight junctions between the endothelial cells that form the capillary walls. While some scientists might argue that the olfactory epithelium (i.e., the mucous membrane surface that contains the olfactory receptor neuron projections, inside the nasal sinuses) can be considered an extension of the brain, and therefore presents a region of CNS tissue where the BBB does not exist, it is nevertheless clear from physiological studies that olfactory glomeruli sit entirely within the BBB, and are protected by the BBB from unwanted molecules that might trigger spurious and unhelpful nerve impulses. Accordingly, to represent that fact, double-dashed line 900 is used in FIG. 1 to schematically represent the BBB. The peripheral projection 210 and main cell body 224 of olfactory receptor neuron 200 sit outside the BBB, while the axon 240 passes through it, and then branches into numerous synaptic terminals 242 which sit inside BBB-protected CNS tissue. Accordingly, the olfactory receptor neuron 200 is referred to herein as a neuron which “straddles” the BBB.

The synaptic terminals 242 of olfactory receptor neuron 200 are located in a roughly spherical globular structure, called a “glomerulus”, shown as region or cluster 910 in FIG. 3. In a human, there are thousands of glomeruli, and each glomerulus contains the synaptic terminals of roughly 25,000 axons from olfactory receptor neurons. Each glomerulus 910 is also the terminus for thousands of dendrites and projections 920 from large “mitral” cells and smaller “tufted” cells. While the cell bodies and dendrites of the mitral and tufted cells are drawn as lying in the glomerulus, their main cell bodies are located in bulb structures positioned above the glomeruli. In addition, each glomerulus (or surrounding regions that are closely proximate to the glomerulus) also contains termini 934 located at the tips of long fibers (called processes) 932 which extend down from neurons 930 that are often called “basal forebrain cholinergic neurons”, since they are located in the basal forebrain, and since they are activated mainly by acetylcholine as the excitatory neurotransmitter.

The schematic representations in FIG. 3 are selective illustrations, and do not attempt to illustrate all targeted neuronal structures. For example, also found in or in the near vicinity of the glomeruli are the termini of: (i) fibrous processes that extend from “serotonergic” neurons in a part of the brain called the raphe nucleus, which have their main cell bodies in a part of the midbrain; and, (ii) other fibrous processes extending from “noradrenergic” neurons which have their main cell bodies in a part of the brain called the locus coeruleus nucleus. Depending on the therapeutic polypeptide being delivered, these or other neurons residing wholly within the BBB might be targeted by a treatment as disclosed herein.

In general, it is anticipated that polypeptides released by a transfected olfactory receptor neuron 200 are likely to be able to contact and exert modulating effects on any (or nearly any) type of neuron that has been shown, using so-called “trans-synaptic tracer studies” (e.g., Lafay et al 1991; Barnett et al 1993), to be infected by virus particles released by virus-infected olfactory receptor neurons. Such neurons that have been shown to be infected in trans-synaptic tracer studies include mitral cells and tufted cells (these are infected in large numbers, since very large numbers of their termini are present in the glomeruli), and at least some basal forebrain cholinergic neurons, raphe nuclei serotonergic neurons, and locus coeruleus noradrenergic neurons (which have also been shown to be infected, in tracer studies).

It should also be recognized that various types of “glial cells” are also likely to be contacted by the foreign polypeptide molecules 300 that are secreted by olfactory receptor neuron 200. As described in more detail below, glial cells (also called neuroglial cells) include various types of cells which cannot receive or transmit nerve signals, and which instead support and serve the neurons that are located inside BBB-protected CNS tissue.

Accordingly, the term “target cells” is used herein to refer to cells which sit entirely within BBB-protected CNS tissue, and which are the intended “targets” of the foreign polypeptide molecules 300 that are encoded by the passenger gene(s) in a genetic vector 100 as described herein. A BBB-straddling neuron which is actually contacted and transfected by a genetic vector is not regarded herein as a target cell; instead, that type of BBB-straddling neuron should be regarded as part of the delivery mechanism, and can be referred to by terms such as “delivery cell”, “transfection conduit”, or as a “primary” transfected neuron.

If olfactory receptor neurons are used as the delivery route, another physiological factor is potentially important, and should be recognized. Olfactory receptor neurons gradually die off, and are constantly being replaced by newly-created neurons. In mice, the “half-life” of olfactory receptor neurons is about 3 months, and the half-life in humans is presumed to be roughly comparable.

Therefore, if olfactory receptor neurons are genetically “transformed” in a stable manner by genetic vectors (i.e., if the genetic vectors cause one or more foreign genes to be inserted into the chromosomes of the olfactory receptor cells), the transformed cells will nevertheless gradually die off over the next weeks and months. As a result, depending on the type of vectors and polypeptides that are involved and the effects that are desired in a particular patient, it may be necessary to readminister additional vectors to the patient, every few weeks.

### NOCIOCEPTIVE NEURONS

Another well-known class of BBB-straddling sensory neurons transmit pain signals from the skin and body, into the spinal cord. These neurons generate nerve impulses in response to impinging substances, signals, or events from the environment; accordingly, these are the neurons that commence the process that generate feelings of pain or discomfort when the skin has been cut, scraped, or hit, or exposed to intense heat or cold. These pain-signalling neurons are usually called “nociceptive” or “nocioceptive” neurons, and are sometimes called nociceptors or nocioceptors. Nociceptive neurons are just one of a number of different functional types of BBB-straddling sensory neurons that form part of the dorsal root ganglia.

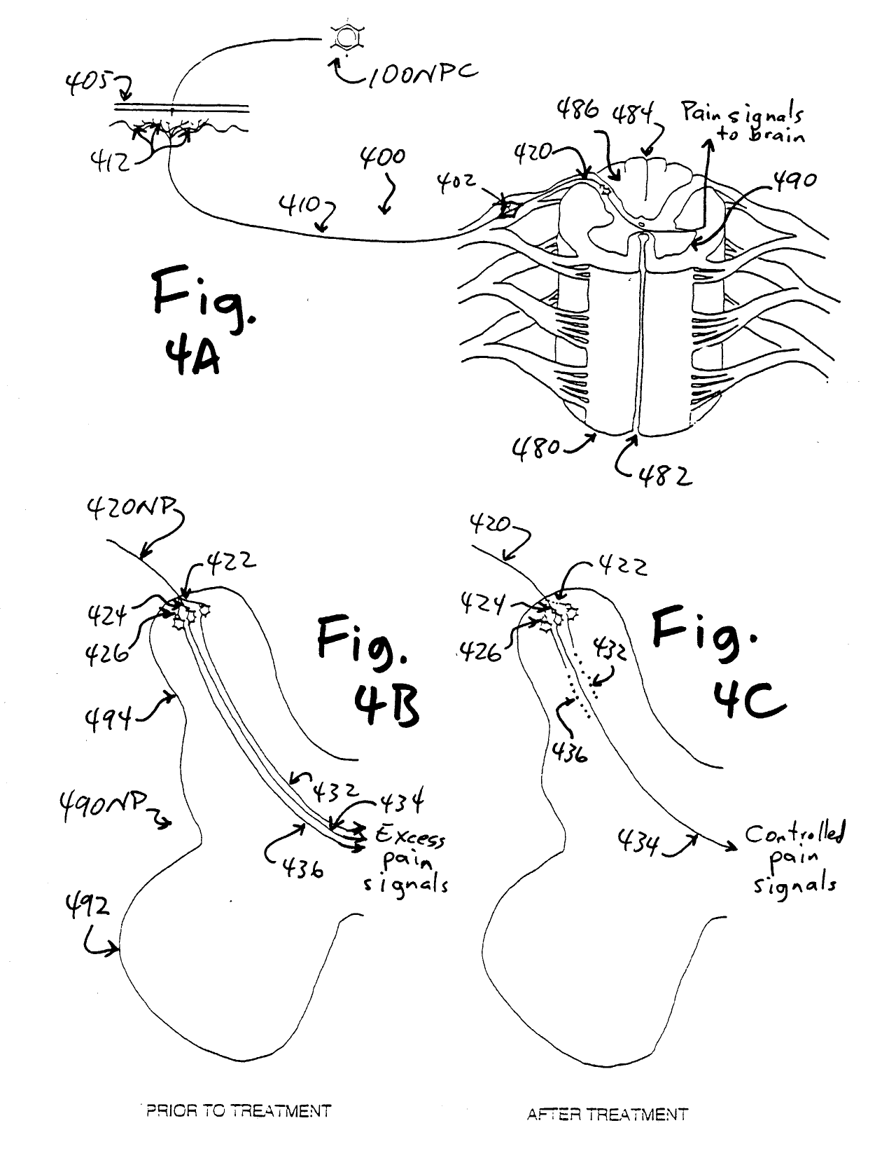

FIG. 4 depicts, in a highly schematic fashion, a nocioceptive neuron 400. Like most other sensory neurons, this neuron 400 has its main cell body 402 in a tissue region which is outside of, and not protected by the BBB; in the case of nocioceptive neuron 400, the main body 402 is located relatively close to spinal cord 480, in a dorsal root ganglion. This neuron has a peripheral projection 410 which extends outwardly, i.e., away from the spinal cord 480, and toward the skin (other projections also extend to other regions deeper within the body). This peripheral projection 410 branches out into numerous “near-surface terminals” 412, located in shallow layers of tissue just beneath the skin surface 405.

In the other direction, nocioceptive neuron 400 also has an axon 420, which passes through the blood-brain barrier. As indicated in FIG. 4B, axon 420 branches into processes 422, 424, and 426 (shown in FIG. 4B), inside BBB-protected CNS tissue in spinal cord 480. Each process terminates in one or more synaptic junctions that allow the nocioceptive neurons to transmit its pain signal to “second order” neurons, inside spinal cord 480.

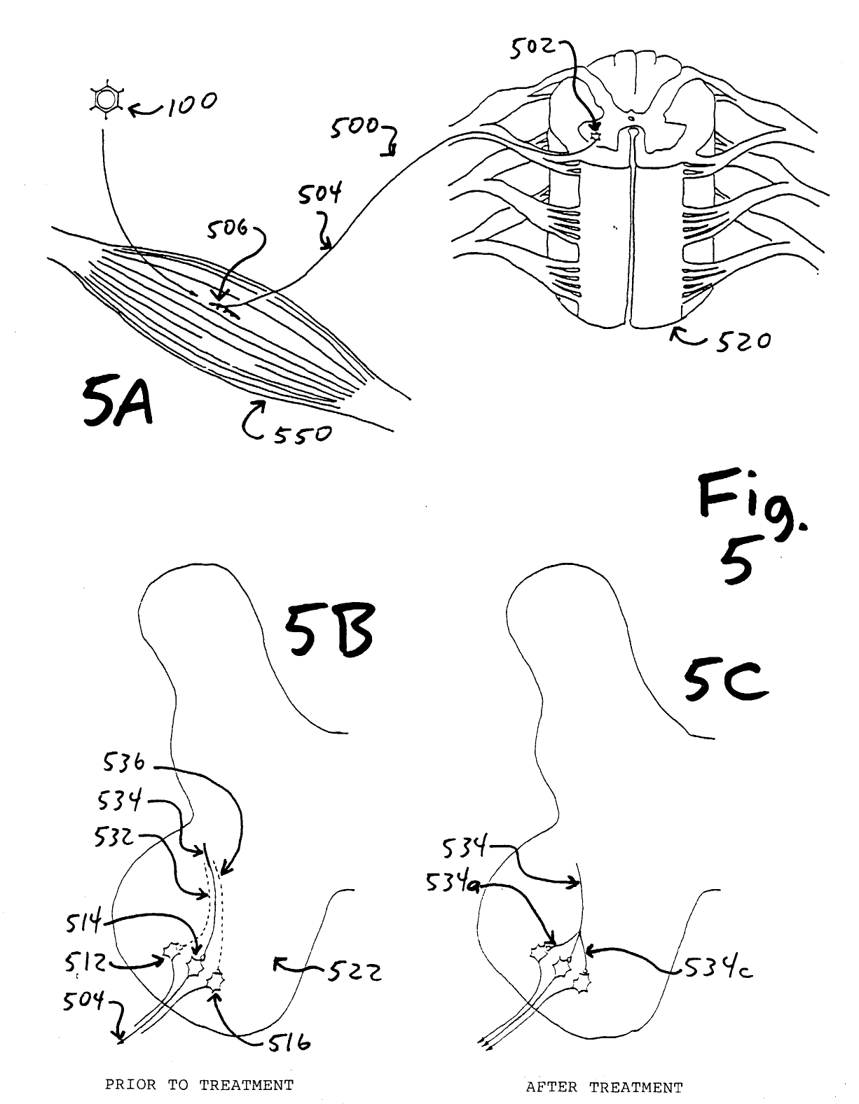

The surface of spinal cord 480, which is shown as a cross-sectioned segment, has: (i) a ventral median fissure 482 (also called an anterior median fissure), positioned toward the front of the patient; and, (ii) a dorsal median fissure 484 (also called a posterior median fissure), flanked by two smaller dorsal-lateral (or postero-lateral) fissures. The mass of spinal cord 480 comprises “white matter” 486 which surrounds “gray matter” 490. The gray matter 490 comprises left and right ventral (anterior) horns 492, and left and right dorsal (posterior) horns 494. These four horns are all connected to a central portion, called the gray commissure. The large bundles of nerve fibers which emerge laterally from the spinal cord, as shown in FIGS. 4 and 5, usually are called the posterior (or dorsal) roots, and the anterior (or ventral) roots; the term “ganglion” (which generally refers to aggregations of neuronal cell bodies outside the central nervous system) can also be used to refer to these nerve bundles.

If skin surface 405 is cut or scraped, the nerve signals that commence at the near-surface terminals 412 of nocioceptive neuron 400 travel through the peripheral projection 410, through the cell body 402, through the axon 420 which crosses the BBB, and into the branched dendrites 422-426 inside the dorsal horn 494 of spinal cord 480. The synapses at the tips of dendrites 422-436 release neurotransmitters, which trigger nerve impulses in the spinal neurons. Those nerve impulses travel up the spinal cord, to centers inside the brain which process the arriving nerve signals in ways that the brain interprets as pain.

Because pain signals are essential to the alert-and-respond mechanisms that animals need and use to protect themselves against serious injuries, they cannot and should not be completely eliminated, in medical practice. However, there are numerous types of medical problems that have been grouped and given the descriptive name, “neuropathic pain.” As indicated by the name, “neuropathic pain” includes any type of pain in which some sort of pathological condition is affecting neurons, in a manner which generates unwanted and excessive pain signals. This often involves some anatomical reorganisation of the nerve connections within the BBB, such that there is a chronic and inappropriate pain response. The term “hyperalgesia” is also used, as a descriptive term that translates directly into “excess pain”, and the term “allodynia” is also used for this condition.

Neuropathic pain is a well-known cluster of medical problems, and this broad category includes diabetic neuropathy, “phantom pain” from limbs or extremities that have been amputated, arachnoiditis, trigeminal pain, post-infective pain (such as outbreaks of “shingles”, caused by herpes zoster viruses), and lingering chronic pain that arises after a traumatic injury or surgery and then will not recede, even after the normal timespan of recuperation has long passed. Other examples of neuropathic pain are also known to physicians, and it should be recognized that neuropathic pain can range over a very wide span of intensity, starting at annoying up the extremes of excruciating and debilitating pain. Indeed, neuropathic pain is involved as a major causative factor in many suicides; chronic and incurable pain can be so intense and relentless that it drives many sufferers to commit suicide.

A cellular mechanism that is believed to be widely involved in many types or cases of neuropathic pain is illustrated in FIG. 4B. In this schematic drawing, axon 420NP has become involved in a neuro-pathological problem which has caused it to sprout too many dendrites 422, 424, and 426, which have begun to interact with spinal neurons 432, 434, and 436 (some of which may be inappropriate) located in the dorsal horn of spinal cord 480NP. This sprouting and chronic activation of too many dendrites from a single nocioceptor axon causes or aggravates the transmission of too many pain signals into and through the pain-plagued spinal cord 480NP. This condition, involving too many neuronal connections involving pain-transmitting neurons, as shown in FIG. 4B, is referred to as “hyper-innervation”.

This invention offers a method of treating (which includes controlling, reducing, suppressing, etc.) neuropathic pain, by administering a genetic vector that can transfect nocioceptor neurons in or near an affected area, as further described herein and illustrated in FIGS. 4A-4C. Subcutaneous, intramuscular, or other relatively shallow injection is a preferred route of administration. Alternately, various other modes of administration also can be evaluated if desired. Such candidate routes might include, for example: (i) topical application of a vector-carrying ointment, cream, or other solution or suspension which contains an agent that promotes permeation through tissue, such as dimethylsulfoxide, methylsalicylate, etc.; (ii) topical application of a vector-carrying formulation to a roughened, abraded, or otherwise physically-treated area of skin; and/or (iii) topical application of a vector-carrying formulation to an area of skin that has been chemically treated, such as by the types of chemicals that are used for “skin-peeling” treatments to remove acne scars, discolored spots, etc.

For purposes of further discussion of neuropathic pain treatments, it is assumed that a liquid containing multiple copies of a genetic vector 100NPC (the letters “NPC” refer to “neuropathic pain control”), as shown in FIG. 4A, will be injected in a shallow subcutaneous manner into a region of skin at or near the location of an apparent “focal point” (also called a locus, seat, hot spot, etc.) where neuropathic pain is perceived most intensely by a patient. Genetic vector 100NPC will carry an “NPC” passenger gene which is designed to suppress (rather than increase) neuronal activity. Such “NPC” genes can encode, for example: (i) monoclonal antibodies (or antibody fragments) that will bind to and inactivate one or more types of neuro-trophic or other neuro-stimulatory peptides or compounds; or, (ii) peptide fragments that will competitively bind to, occupy, and block one or more types of neuronal cell receptors that are involved in neuro-stimulatory processes. The NPC gene will be transported (using retrograde transport, as noted above) to the cell body 402 of nociceptor neuron, where the gene will be expressed to form copies of an NPC polypeptide such as described above. The NPC polypeptides will then be transported (using anterograde transport) by the neuronal axon 420, across the BBB and into the spinal cord 480NP which is being plagued by neuropathic pain which is being caused or aggravated by the hyper-innervation condition shown in FIG. 4B.

The NPC polypeptides will then be released into the BBB-protected spinal tissue, at or near the site of the hyper-innervation condition. By exerting their suppressive effects (such as by binding to, blocking, competing against, or otherwise suppressing cellular agents or processes that stimulate or sustain higher levels of neuronal activity), the NPC polypeptides will help reduce and mitigate the neuropathic pain condition, either on a permanent basis, or on a basis that may last for days or weeks, depending on what type of treatment is used.

### MOTOR NEURONS

Another class of BBB-straddling neurons (in addition to the various types of sensory neurons) is usually called motor neurons. These neurons transmit instructions from the CNS principally to the muscles of the body, to “innervate” skeletal musculature and place the muscles under the control of the CNS. One major subdivision of motor neurons, usually called “spinal motor neurons”, have their cell bodies in the spinal cord, and projections which extend out through the BBB to contact peripheral nerve cells and muscle.

FIG. 5 (which comprises FIGS. 5A-5C) schematically depicts the use of a spinal motor neuron as a “transfection conduit” (as described above) to stimulate and increase neuronal control over a muscle, in a patient who has a weakened limb due to a stroke, traumatic injury, neurodegenerative disease, or similar cause, by delivering therapeutic polypeptides to upper motor neurons that lie wholly within the BBB. FIG. 5A depicts spinal motor neuron 500, which has a cell body 502 located inside BBB-protected tissue in spinal cord 520. Spinal cord 520 has the same structure shown in FIG. 4A, and the motor neuron cell body 502 is located inside the gray matter, in ventral horn 522. The axon of spinal motor neuron passes through the BBB, and forms a long “process” 504 which extends to muscle fiber bundle 550. Inside the muscle fiber bundle 550, the neuronal axon or process 504 branches into a number of dendrites and terminals 506, which (in a healthy person) interact with the muscle tissue to trigger muscle contractions at desired times, thereby providing “voluntary motor control” of the arms, legs, etc.

FIG. 5B is a schematic depiction of the cell bodies of three distinct spinal motor neurons, designated as 512, 514, and 516, in a patient who has suffered a neurological crisis such as a stroke or a head or spinal injury, or who is suffering from a neurodegenerative disease (such as amyotrophic lateral sclerosis, ALS) or a similar problem. The neuronal damage suffered by this patient has damaged or destroyed his voluntary motor control over an arm or leg. This impaired condition is due, at least in part, to dead or damaged upper motor neurons in the brain or brainstem, above the location of the three motor neuron cell bodies. Some of the dead or damaged upper motor neurons in the brain or brainstem, above the location of spinal motor neuron cell bodies 512-516, had supplied nerve impulses to spinal motor neurons 512 and 516; as depicted schematically in FIG. 5B, when various upper motor neurons were damaged or killed, their processes 532 and 536 (indicated by dotted lines in FIG. 5B) fell silent and began to degenerate, leaving only one of the three spinal motor neurons (illustrated as the center process 534) with an active supply of incoming nerve signals, from its upper motor neuron. Since the two motor neurons 512 and 516 are no longer receiving any incoming nerve impulses, they have fallen into silence and disuse, and are in danger of atrophying, deteriorating, and dying over time.

FIG. 5C schematically depicts the result of treatment of this condition, following injection of a liquid containing copies of genetic vector 100 into the muscle bundle 550 that is no longer adequately functioning. Vector 100 carries a therapeutic gene which encodes a neurotrophic factor or other polypeptide that stimulates neuronal activity or replication. The gene carried by this vector is transported into the cell bodies 512-516, where the gene is expressed into neuro-stimulating polypeptide molecules. These polypeptides are then secreted by the three motor neurons, and they act as signals which stimulate and attract the growth and/or activation of additional dendrites from any nearby neurons which are still viable and active, including the upper motor neuron having the active process 534. This causes the active process 534 to sprout additional processes 534a and 534c, which can form synapses with other neurons. The additional newly sprouted synaptic junctions thereafter begin activating the processes from spinal motor neurons 512 and 516 once again. When coupled with a physical therapy and exercise program, this restores to the patient a greater degree of voluntary control over his arm or leg.

One particular subclass of lower motor neurons which may be highly useful in this invention has projections which extend through the BBB to innervate the muscles of the tongue. These “tongue motor neurons” control the movement of the tongue, for both eating and talking. They are mentioned in specific, because their cell bodies are located within the brainstem; therefore, they offer a promising passageway for delivering foreign polypeptides into the brainstem portion of the CNS.

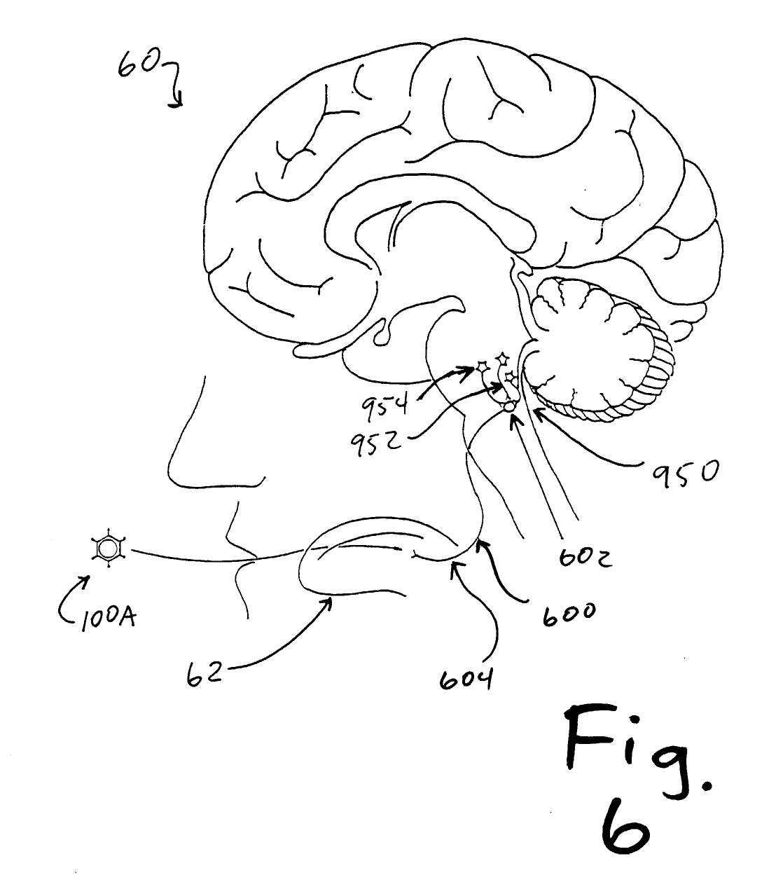

This is depicted in FIG. 6, which depicts a tongue motor neuron 600, having a cell body 602 located in the brainstem 950, and a long peripheral projection 604 which passes through the BBB and terminates inside the tongue 62 of patient 60. The accessible tip of neuronal projection 604 is contacted by a genetic vector 100A, by means of a carrier liquid injected into the tongue 62. The vector DNA will be retrograde-transported through the neuronal projection 604, into the cell body 602. The passenger gene will be expressed into a therapeutic polypeptide, which will be secreted by the tongue motor neuron 600, at locations in the brainstem. These secreted polypeptides will contact and exert their effects on various other neurons 952 and 954, located in brainstem 950.

### PRE-GANGLIONIC AUTONOMIC NEURONS

The third major class of BBB-straddling neurons is usually called pre-ganglionic autonomic neurons. Like motor neurons, their cell bodies are located inside the BBB. Their axons extend out through the BBB, and connect with nerves of the sympathetic and parasympathetic nervous systems. As part of the “autonomic” system, these neurons are involved in the control of various functions that are not under conscious control (such as blood pressure, digestion, excretion, sweating, etc.).

The term “ganglion” implies a bundle of neurons. Accordingly, “pre-ganglionic” neurons include neurons whose cell bodies are found within the CNS in clusters (also called nuclei) and whose axons project through the BBB to innervate and make contact with the neurons found in the ganglion lying outside the BBB.

It is believed that the methods and genetic vectors of this invention can be adapted and used, if desired, to genetically transfect pre-ganglionic autonomic neurons, and it is believed that in at least some cases, such transfected pre-ganglionic autonomic neurons will subsequently transport and deliver the foreign polypeptides into the brainstem and/or spinal cord, and possibly other CNS neurons lying wholly within the BBB (for generally supporting data, see, e.g., Pickard et al, 2002). However, sensory neurons and motor neurons are-likely to be somewhat easier to work with and evaluate, at least during the early stages of development of this invention. This is due to several factors. On one hand, the projections of sensory and motor neurons actually reach (or closely approach) exposed and accessible surface locations (in the case of olfactory neurons), and relatively shallow muscle and subcutaneous regions (in the case of pain-transmitting and motor neurons); by comparison, pre-ganglionic autonomic neuron terminals are buried substantially deeper beneath the skin surface, within the autonomic neuronal ganglions. Therefore, pre-ganglionic autonomic neurons are likely to pose somewhat greater technical challenges (and somewhat greater risks) for vector delivery than sensory or motor neurons, if used as BBB-straddling “transfection conduits” as disclosed herein.

Therefore, the examples and most of the discussion herein focus on using sensory neurons or motor neurons, as the BBB-straddling neurons that are accessible to transfection by injectable genetic vectors, to describe and illustrate the invention herein. Those classes of BBB-straddling neurons are believed to provide generally preferred candidates for initial development and testing of this invention. Nevertheless, pre-ganglionic autonomic neurons should be recognized as having potential use as BBB-straddling “transfection conduits” as disclosed herein, and may eventually become highly useful for various types of therapeutic or other treatments.

Genetic Vectors

As summarized above, genetic vectors (such as viral vectors, liposomes, ligand vectors that target endocytotic receptors on BBB-straddling neurons, etc., as described in more detail in the Examples) that carry one or more useful “passenger” genes (which can include “marker” genes and/or “payload” genes) are contacted with the peripheral projections of BBB-straddling neurons, in a manner which promotes transfection of vector DNA (including the useful passenger gene) into a BBB-straddling neuron. Once inside such neuron(s), at least some copies of the passenger gene(s) will be transported through the projection to the main cell body, by a natural process called “retrograde transport”. Once inside the cell body, the passenger gene(s) will be expressed by the normal intracellular mechanism, to create the gene-encoded polypeptide.

Since most polypeptides of interest herein (such as neurotrophic growth factors) act as hormones or growth factors, which normally and naturally must be secreted by cells in order to carry out their necessary functions, such polypeptides, created in transfected BBB-straddling CNS neurons, will already be well-suited for secretion by the transfected neurons.

Accordingly, this invention discloses a class of genetic vectors that can be used to transfect certain types of selected CNS neurons which straddle the BBB, in a manner which causes the transfected neurons to express and then secrete useful and therapeutic polypeptides, into CNS tissue that normally is protected from foreign polypeptides by the blood-brain barrier.

FIG. 2 shows a schematic depiction of a viral vector 100. This vector can be regarded as having three primary components: an encapsulating portion 110, binding ligand proteins 120, and genome 150 (which, in FIG. 2, is shown as double-stranded DNA, or dsDNA).

In viral vectors which do not have lipid “envelopes” (such as vectors derived from adenoviruses), the encapsulating portion 110 is made of capsid proteins 112, which fit together in a semi-interlocking manner. In such vectors, the protein “shell” is usually called a capsid, and the binding ligand proteins 120 usually are nothing more than capsid protein domains which are exposed on the exterior surface of each viral particle.

In other types of viral vectors, such as herpes viruses, the encapsulating portion (usually called an “envelope”) is made of lipids, usually arranged in a bilayer form which is comparable to the lipid bilayers that make up the outer membranes of most mammalian cells. In such vectors, the binding ligand proteins 120 usually straddle the envelope layer, and a protruding external portion extends outwardly and contacts cells.

Regardless of whether a lipid envelope is present, the viral binding ligands 120 adhere (in a non-covalent manner, comparable to an antibody-antigen binding reaction) to complementary proteins on the surfaces of cells which are susceptible to infection by that type of virus. Some types of viruses (and vectors derived from them) can have binding ligands that are highly specific; these types of viruses can infect only certain types of cells, which have complementary surface proteins. Other types of viruses (and vectors derived from them) have binding ligands that are much less specific, and can bind to and infect a much wider variety of cells.

The vector genome carried by the vector 100 shown in FIG. 2 comprises double-stranded DNA (dsDNA). As known to those skilled in the art, other types of viral vectors can carry single-stranded DNA, or single- or double-stranded RNA. Various genetic vectors with all four categories of genomes are known, and any such vector can be evaluated for use as disclosed herein, using no more than routine experimentation. In general, viral vectors carrying dsDNA genomes are regarded as likely to offer preferred candidates for early evaluation of this invention, for two reasons: (i) various categories of viruses which offer the most initially promising vector vehicles that can infect neurons (this group includes herpes viruses, and adenoviruses) contain dsDNA genomes; and (ii) during laboratory manipulation of synthetic or isolated genes, it tends to be easier to work with dsDNA than with ssDNA (which tends to be “sticky” due to the innate attraction of the bases for each other) or RNA (which tends to be somewhat less stable than DNA).

Using conventional terminology that has previously been developed for use with viral vectors, genome 150 carried by a viral vector (and by various other types of genetic vectors as well) can be regarded as comprising a number of “domains”. One or more “passenger genes” 160 will most commonly be inserted somewhere into the middle of the viral genome, in order to ensure that both ends of the viral genome can function properly once the viral genome enters a transfected cell. Accordingly, the insertion of passenger gene 160 into the center of a viral genome results in creating two “flanking sequences” 155 and 157, containing native or modified viral DNA sequences which flank both ends of the passenger gene. The tips of the flanking sequences 155 and 157 frequently will be coupled to viral polypeptides 158 and 159; in general, these types of polypeptide “caps” evolved to help viral DNA remain relatively stable and resistant to the defensive mechanisms inside a cell that will attempt to chew up and dismantle the viral DNA after it invades the cell. If desired, either or both these polypeptide “caps” can be engineered to promote and speed up various cellular processes, such as retrograde transport, active transport of the viral DNA into the cell nucleus, etc.

The “passenger gene” 160 can also be regarded as having three distinct domains. The promoter region 162 contains a signalling sequence, which directs “transcriptase” enzymes to get ready to begin transcribing that strand in the DNA double helix, to form strands of messenger RNA. This promoter region normally contains a so-called “TATA” box or similar signal, which directs the enzymes to begin transcribing mRNA from the DNA sequence, at a location which is usually about 25 bases downstream from the TATA box. For simplicity of discussion herein, that sequence of about 25 bases, located between the TATA box and the first base which is transcribed into mRNA, is regarded herein as part of the gene promoter. Alternately, if preferred, it can be referred to as part of a “non-transcribed leader sequence”, which would not need to be regarded as part of the actual gene promoter.

The gene promoter is then followed by a “coding” region 164, which determines the sequence of the bases in the mRNA strand that will be transcribed from that gene. This region includes the DNA sequence that specifies an AUG “start” codon in the mRNA strand (which specifies a methionine residue as the first amino acid at the N-terminus of the polypeptide), and a “stop” codon (which truncates the translation of the mRNA strand by the ribosomes). In some vectors, the coding region in a passenger gene may also contain introns, which will be deleted from the final mRNA strand by “editing” processes inside the host cell.

The coding region is then followed by a “non-translated” sequence 166. This domain will be transcribed and will be part of the mRNA strands that are created inside a host cell. These domains, when present on mRNA strands, help stabilize the mRNA strands inside host cells, and protect them against rapid degradation by enzymes. However, this non-translated sequence is not translated into a polypeptide sequence, by ribosomes.

Variations on this general genetic structure are possible and may be used in this invention. As just one example, after the coding region 164 for one polypeptide and before the non-translated sequence 166, an “internal ribosome reentry site” (IRES), such as derived from a RNA virus such as encephalomyocaditis virus, may be inserted with a second coding sequence to allow co-expression of a second polypeptide (e.g., Wong et al 2002). The IRES instructs ribosomes to bind to the mRNA segment in a second location, and to commence expressing a second polypeptide in parallel with the first.

Viruses have evolved various different ways of introducing their DNA (or RNA) into cells, and genetically engineered viral vectors can make use of the same types of infective processes to deliver their DNA “payload” into susceptible cells.

As one example, in a process that is fairly common process among viruses that do not have lipid envelopes, a virus's capsid proteins will attach to protein molecules that are displayed on the host cell's outer membrane. This attachment process initiates a process called “invagination”, which results in the virus being drawn into and becoming packaged within a lipid bilayer envelope, or vesicle, that is formed entirely of lipids from the cell's membrane, but which is now located inside the cell, suspended in its cytoplasmic fluid. This bubble-like vesicle, often called an “endosome,” subsequently ruptures, either with the aid of the virus capsid proteins, or due to digestive processes or organelles inside the cell's cytoplasm. This rupturing of the endosome liberates the viral DNA into the cell's cytoplasm.

In another example, in a process which is fairly common among herpes viruses and other viruses that have lipid bilayer envelopes, the different lipid bilayer membranes which enclosed the virus and the cell become fused together, and effectively merge with each other. This results in the entire contents of the virus's lipid envelope being transferred into the cell cytoplasm.

Regardless of which infective method is used by a selected type of viral vector, the result is that an effective viral vector transfers some or all of its genetic material into the cytoplasmic fluid of a cell. Returning to the example illustrated in FIG. 1, viral vector 100 inserts its DNA 150 into the exposed mucosal tip 212 of olfactory receptor neuron 200.

In some cases, after this type of genetic vector carrying a useful passenger gene has transfected neurons which straddle the BBB, any of several other fates and effects can result, depending on the replication and transmission traits of the vector system that was used. In most cases, it is assumed that the vector and passenger genes will remain inside transfected BBB-straddling neurons, and polypeptides expressed by the passenger genes in those transfected neurons will simply be secreted by the transfected neurons. However, some types of vectors (which are referred to in the examples as “trans-neuronal” vectors) may themselves be secreted by the BBB-straddling neurons, at locations inside the BBB; this would allow such trans-neuronal vectors (or their genetic material) to contact and transfect other neurons which are entirely inside the BBB.

It also should be noted that genetic vectors and gene constructs developed for use as disclosed herein can use any of various techniques and DNA sequences that are known for expressing higher quantities of an encoded polypeptide. Several such techniques and sequences are discussed below, under the subheading, “Gene and Vector Constructs; Expression, Transport, and Secretion Enhancers”.

It also should be recognized that “marker genes” (also called “reporter genes”), which can allow easier, faster, less expensive, more reliable, or otherwise enhanced detection, quantification, and/or isolation of transformed cells and cellular pathways and fates, offer an important category of passenger genes which can be carried by genetic vectors as disclosed herein. Such genes, which are well known in the art of genetic engineering, can allow facilitated and improved research, evaluation, and development in various fields of industry, commerce, and medicine, in various ways that will be readily apparent to those skilled in the art.

Additional comments on various types of genetic vectors are contained in the Examples, below.

### CANDIDATE THERAPEUTIC POLYPEPTIDES

A wide assortment of potentially therapeutic or other useful polypeptides can be introduced into CNS tissue, by using BBB-straddling neurons in ways that can be regarded as conduits, passageways, delivery agents, etc. To illustrate just one area of application (i.e., therapeutic treatment of various known neurodegenerative or neuropathic diseases, in humans) in somewhat more detail, a partial list of polypeptides with known activities (and therapeutic potential) in the CNS is shown in Table 1, and Table 2 provides a very brief and partial listing of various CNS disorders, correlated with polypeptides that are currently thought to have promise in treating or preventing these disorders, and BBB-straddling neurons that may be used to deliver those polypeptides to the target neurons located entirely within the BBB. A number of Examples are provided, below, which discuss how such polypeptides may be administered therapeutically, with the aid of genetic vectors as disclosed herien; since those Examples are relatively complex, a narrative summary and overview is provided below, as a preface to the Examples. This discussion is not an exhaustive list of medical problems that can be treated therapeutically by this invention, and those who are skilled in neurology and neuropharmacology will recognize other potential uses and therapies that may become feasible, after the methods disclosed herein have been made known to the public.

1. Various neurotrophic factors, growth factors, or neurite inhibitory factors, such as listed in Table 1, may help prevent or repair various forms of neuronal damage caused by CNS disorders such as neurodegenerative diseases, or by ischemic or hypoxic crises such as stroke, cardiac arrest, suffocation, blood loss, or other types of physical injury or trauma.

2. Some types of neurotrophic hormones, growth factors, or neurite inhibitory factors can help stimulate the formation of new synaptic connections between existing neurons and/or guide the outgrowth of neuronal processes to facilitate some connections and discourage others. In some patients, this type of treatment can help facilitate the recovery of nervous function loss due to aging or various diseases. It may also help patients regain muscular, speech, and other functions after a stroke, head injury, or other ischemic, hypoxic, excitotoxic, or similar crisis.

3. Various types of endocrine, paracrine, and related or similar polypeptides can help treat various glandular, growth-related, maturation-related, sexual, and other disorders.

4. Polypeptides that can increase the quantities of certain neurotransmitter molecules inside the BBB can treat various neurodegenerative diseases. For example, polypeptides that can increase dopamine levels inside the brain (by acting as enzymes, hormones, or release factors, or through various other mechanisms) can be used to treat Parkinson's disease. Alternately, polypeptides that can increase acetylcholine levels can be used to treat Alzheimer's disease.

5. Cytotoxic or growth-suppressing polypeptides can be used inside the BBB to treat cancer or certain other diseases.

6. Various types of receptor antagonists, antibodies, and other polypeptides that can block or suppress one or more types of neuronal activity can be used to help control and reduce neuropathic pain, hyperalgesia, and similar problems.

7. Lysosomal storage diseases due to lack of a particular polypeptide in the CNS may be treated by delivery of that polypeptide into the CNS.

8. Infections of the CNS by viruses, prions, or bacteria may be treated by delivering into the CNS that help control or reduce the spread of the infection. For example, delivery of polypeptides that bind to the receptors and inhibit virus docking can reduce the spread of viruses such as HIV within the CNS.

9. Delivery of recombinant antibodies to antigens within the CNS can be used to modulate physiological processes in a beneficial or useful way. For example, delivery of recombinant antibodies to myelin associated neurite inhibitory molecules such as No-Go may be able to enable regrowth and regeneration of CNS nerves following spinal cord injury and other traumatic injuries.

### TARGETED CNS NEURONS OR GLIAL CELLS

The term “target cell” is used to refer to a neuron or glial cell that: (i) is part of the CNS, and lies wholly within the BBB; and (ii) is contacted by, or is intended to be contacted by, an exogenous polypeptide that has been delivered into BBB-protected brain or spinal tissue by a method and vector as disclosed herein.

It should be noted that this term deliberately excludes BBB-straddling neurons, even though such neurons can be regarded and referred to by terms such as “primary” or “initial” targets of the genetic vectors that will be used to contact and transfect such cells. In the overall scheme of the invention disclosed herein, the transfection of such BBB-straddling “initial targets” will be valuable and useful, only insofar as that step in the multi-step process will later lead to the subsequent delivery of vector-encoded polypeptides into CNS tissue that is protected by the BBB. Accordingly, the real “targets” of this invention are neurons or glial cells that are entirely within, and protected by, the BBB.

Any references herein to “glial” cells arises from the fact that, within BBB-protected brain and spinal tissue, cells are divided into two categories, referred to as neurons, and glial cells (also called “neuroglia cells” in some medical texts). By definition, the term “neuron” is limited to cells that can receive and transmit nerve signals. The term “glial cells” is a broader residual term, and it includes all types of CNS cells that cannot receive or transmit nerve signals. These glial cells perform various activities that can be regarded as supporting, housekeeping, and “nursing” functions within the CNS; this helps neurons do their essential work. The word “glia” comes from the same root word as “glue”; glial cells were initially thought of as the “glue” that holds CNS tissue together. Glial cells are divided into various categories, including oligodendroglia cells, astrocytes, ependymal cells, and microglia cells. They are discussed in nearly any textbook on neurology, and are a crucial part of the CNS.

For several reasons, the initial work to develop this invention is likely to focus upon using exogenous polypeptides to contact and modulate neurons, rather than glial cells. One major reason driving that trend is that it is likely to be much easier to confirm and quantify CNS responses that directly involve neurons, as compared to effects and responses that involve glial cells first, and affect neurons only as a secondary effect.

Accordingly, while some researchers will prefer to evaluate this invention by focusing on “target cell” neurons lying entirely within the BBB, it should be recognized that: (1) the methods and vectors of this invention may also be useful for providing ways to treat CNS tissue by using exogenous polypeptides to contact and treat glial cells; and (2) certain specific conditions involving glial cells are likely to merit relatively early research and evaluation, such as potential treatments for certain types of glial cell cancers (including glioblastomas and astrocytomas), and methods for modulating the responses of glial cells to traumatic injury, or hypoxic or ischemic insult.

As implied by the term “target”, not all neurons or cells wholly within the BBB will necessarily make contact with polypeptides delivered using the invention. Where a BBB-straddling neuron (or cluster of neurons) is transfected by a genetic vector, the “target” neurons or glial cells lying wholly within the BBB will be either: (i) positioned in close proximity to the BBB-enclosed synapses or other terminals of transfected neurons; or, (ii) have cell processes or extensions (such as dendrites, axons, or terminals) that are in close proximity to the BBB-enclosed synapses or other terminals of transfected neurons. Where a “transneuronal” vector is used (i.e., where the genetic vector itself will be able to travel through a BBB-straddling “primary” neuron, to a second- or third-order neuron that is located inside the BBB, and that will be transfected by the vector so that it will subsequently express the encoded polypeptide), “target” neurons or glial cells may be located in close proximity to second-order or third-order transfected neurons which will express and secrete polypeptides encoded by vector-borne foreign genes.

As examples of how one or more classes of neurons inside the brain can become “target” neurons inside the BBB, basal forebrain cholinergic neurons, serotonergic raphe neurons, and noradrenergic locus coeruleus neurons do not have direct synaptic junctions with BBB-straddling olfactory receptor neurons; nevertheless, these classes of cholinergic, serotonergic, and noradrenergic neurons inside the BBB can be target neurons, if olfactory neurons are transfected by a genetic vector as disclosed herein, because they can take up polypeptides released by transfected olfactory receptor neurons. This is illustrated by the fact that these classes of cholinergic, serotonergic, and noradrenergic neurons inside the BBB can be infected by rabies or herpes simplex viruses released from virus-infected olfactory receptor neurons (Lafay et al 1991, Barnett et al 1993).

The principle that CNS neurons or glial cells can be “targeted” for contact and treatment by exogenous polypeptides, if they are positioned inside the BBB adjacent to or within fairly close proximity to a central projection of a BBB-straddling cell, can be used most advantageously if supported by the best currently available information on the anatomical relationships between transfected and targeted cells. This type of anatomical knowledge can be found in the technical literature where investigators have made use of viruses or other “transneuronal labelling” molecules. For example, if a particular class of CNS neuron or glial cell has been shown to become infected with a transneuronal virus, following application of that type of virus to BBB-straddling neurons in lab animals, the subsequently-infected class of CNS neurons or glial cells can be used as target neurons or glial cells for the purposes of this invention. This type of neuronal mapping has already been carried out to some degree by various researchers, and is described in references such as Loewy 1998 and Norgren et al 1998. In addition, this type of work continues to this day, and as it continues to provide more information on neuronal circuitry, that additional information can be taken into account by anyone practicing this invention.

The preferred method for contacting a genetic vector with a peripheral projection of a sensory or motor neuron will depend on the structure and location of the targeted peripheral projection. For example, administration to olfactory receptor sensory neurons can be via nasal instillation, such as by using a nasal spray, or by using a liquid-saturated packing material that can be placed in the nasal sinuses, in direct contact with the nasal surface area which contains olfactory projections, for some period of time (such as 30 to 90 minutes).

If desired, direct and sustained contact between a gene vector and the olfactory neuron projections can be further promoted by steps such as (i) using a nasal decongestant to reduce and minimize any mucous covering the nasal sinus surfaces; (ii) using a preparation of a cleaning or similar agent, such as dilute isopropyl alcohol, acetone, etc., to further clean and prepare the area to be contacted; and/or (iii) using a mechanical scraping procedure, as is commonly performed by otolaryngologists to treat patients with recurrent nasal sinus infections. In general, neuronal projections in a surface area which has become irritated to a point of mild inflammation tend to be more receptive, to cellular uptake of foreign molecules, than cells which can be regarded as being in a quiescent or resting state.

If desired, neuronal contact and uptake can be further promoted and increased by means of various compounds that are referred to as “muco-adherents”. Such compounds are being actively developed for use in increasing the delivery of various drugs into the bloodstream, via transmembrane routes such as through nasal sprays. Example of muco-adherents that are being developed for this purpose include chitosan (discussed in articles such as Schipper et al 1999) and various types of polysaccharide colloidal preparations (discussed in articles such as Janes et al 2001); also see Rillosi et al 1995 and Lim 2000 for further discussion of muco-adherents.

In addition, it may also be possible to further stimulate entry of the genetic vector into the neuronal projections by means of an electrical charge or surge applied to the nasal surface, in a manner comparable to the in vitro transfection technique known as “electroporation”. If desired, any or all of these steps can be carried out while a patient is under general anesthesia, or under a mild local anesthesia.

Administration to projections which are part of nocioceptive neurons can be via cutaneous or sub-cutaneous injection, by various controlled skin abrasion techniques, and possibly by topical application in some cases, if adequate penetration can be achieved. If motor neuron projections are used as the access route, administration usually will require a sub-cutaneous or intramuscular injection. For example, projections which are part of the lower motor neurons of the hypoglossal nucleus can be accessed by injecting a genetic vector into the muscles of the tongue. Although in some cases, gene vectors may be administered via an intravenous route (especially if a particular nerve-targeting component is included), it is generally believed to be preferable to administer gene vectors by intramuscular or similar injections that will establish and sustain high concentrations of vectors at desired targeted locations.

This also points out a significant difference between this mode (involving targeted transfection of selected neurons), and delivery of genes, polypeptides, or other compounds via injection into cerebrospinal fluid. Physical delivery into the cerebrospinal fluid system (such as via catheter into the ventricles of the brain or intrathecal space of the spinal cord) will distribute a neurologic agent to a large number and array of CNS cells. In contrast, this invention describes how delivery of desired therapeutic polypeptides can be targeted to, or result in controlled and preferential delivery to, only limited number of cells or neuronal processes, which are in close contact with (or synapsing with) transfected BBB-straddling neurons.

It is anticipated that the same procedures and classes of genetic vectors disclosed herein can be adapted and used, if desired, to introduce polypeptides into the brains and spinal cords of non-human mammals, and into other classes of animals that have blood-brain barriers, including reptiles and birds. As such, this invention may well become useful for controlling and regulating the rate of growth and/or reproductive status of livestock, pets, and other animals. For this and other purposes, hypothalamic releasing factors can be delivered via the olfactory receptor neurons to regulate the release from the pituitary gland of potent hormones. For example, GHRH may be delivered to stimulate release of growth hormone to accelerate an animal's growth rate. Also, sustained delivery of GnRH at supramaximal dose may be used to inhibit normal release of LH and/or FSH release, in a manner which may achieve or promote contraception, including reversible contraception. If the methods disclosed herein are being adapted to other mammalian species, or to other classes of animals such as birds and reptiles, the primary adaptations that will need to be made include (i) selection and use of genetic vectors that are well-suited for transfecting the projections of BBB-straddling neurons in the chosen animal type, and (ii) selection and use of a polypeptide type which has the desired activity in that particular species of animal.

Without disregarding these and other potential applications, the remainder of the discussion herein focuses solely on animal tests to prove the methods and procedures disclosed herein, and on treating humans for medical purposes.

### VIRAL VECTORS

Review articles that describe various types of mammalian viral vectors include Karpati et al 1996, and Kaplitt and Makimura 1997. At least four types of viral vectors have been used to transfect neurons. Those virus types are:

### A. Adenoviruses

These are double-stranded DNA viruses that are often found in various glands; wild-type viruses cause respiratory infections, conjunctivitis, and various other problems. Genetically-engineered adenoviruses that have been rendered incapable of replicating (except in special types of cells and/or culture media that exist only in laboratory conditions) have become the main class of viral vectors used in in vivo studies on mammals, including gene therapy efforts on humans. Adenovirus vectors have been used to transfect various types of neurons, as reviewed in Smith and Romero 1999.

### B. Adeno-Associated Virus (Dependovirus; Adenosatellite virus)

These are single-stranded DNA viruses that depend on Adenoviruses for replication. Methods for preparing adeno-associated virus vectors can be found in the chapter by Bartlett and Samulski, in Robbins (ed.) 1997.

### C. Herpes Simplex Viruses (HSV)

These are double-stranded DNA viruses, with capsids that are surrounded by lipid envelopes. Use of engineered HSV vectors to transfect neurons is discussed in Staecher et al 1998.

### D. Retroviruses, Including Lentiviruses

These viruses contain RNA, rather than DNA. Use of a lentivirus vector to deliver and express the GDNF gene into lower motor neurons of mice was described in Hottinger et al 2000.

These four classes of viruses appear to be receiving the most effort and attention at this time, in attempts to create viral vectors that can transfect cells, but which are generally nonpathogenic after they enter their target cells (usually due to deletions or defects in one or more genes which encode proteins required for replication). However, other classes of known neurotropic viruses (which includes numerous types of viruses that cause viral encephalitis, including some RNA viruses) offer promising candidates for vectors that can selectively (or at least preferentially) transfect neurons. If any of those classes of viruses can be rendered safe and non-pathogenic by means of genetic manipulation comparable to the steps used to render other viral vectors non-pathogenic, they may be well-suited for use as disclosed herein.

Alternately or additionally, using genetic engineering techniques, various types of viral vectors that do not have a strong affinity for neurons can be provided with selected and/or modified surface proteins (including chimeric surface proteins) that bind preferentially to surface proteins on the projections of olfactory neurons, motor neurons, or other types of neurons that straddle the BBB. If properly developed and used, modified viral surface proteins with increased neuron-binding affinity may increase the speed, efficacy, and other benefits of viral vectors when used as disclosed herein.

### NON-VIRAL VECTORS

As briefly summarized in the Background section, and as discussed in more detail in various examples below and in numerous articles and review articles on the subject, several types of known non-viral genetic vectors can be evaluated for use as disclosed herein. Primary candidates for classes of nonviral vectors which can transfect neuronal projections include cationic liposomes, and protein-DNA complexes containing polypeptide ligands that bind to endocytotic receptors on neurons.

Numerous methods and tricks are known to those skilled in the art, for increasing the likelihood that a non-viral vector will succeed in accomplishing its intended results. As just one example, a neuron-targeting non-viral vector was recently reported which makes use of neurotensin, to target plasmid delivery to neurons of the nigrostriatal and mesolimbic dopaminergic systems (Martinez-Fong et al 1999). Various other examples are discussed in the following sections.

In general, plasmid vectors constructed for use with a cationic liposome or receptor-mediated endocytosis delivery mechanism will contain both: (i) sequences to enable replication of the plasmid in a host cell, such as E. coli, and (ii) sequences that enable expression of the gene for a therapeutic polypeptide in target cells. For replication, the vector ordinarily carries a replication site, as well as marker genes that allow simple selection of transformed cells. For example, for replication in E. coli, the plasmid pBR322 (Bolivar et al 1977) contains genes for ampicillin and tetracycline resistance, which allow quick and simple identification of transformed cells by using those antibiotics.

The sequences that enable expression of the gene for a therapeutic polypeptide in a target cell ordinarily include an origin of replication (if necessary), a promoter located in front of the gene to be expressed, along with any necessary ribosome binding sites, RNA splice sites, a polyadenylation site, and transcriptional terminator sequences.

### GENE AND VECTOR CONSTRUCTS; EXPRESSION, TRANSPORT AND SECRETION ENHANCERS

A variety of known genetic engineering techniques and DNA or polypeptide sequences can be used to improve and increase: (i) the likelihood that this method will successfully accomplish a detectable level of a desired and intended result, in any particular animal or patient; (ii) the potency, efficacy, duration, or other desired aspects of the treatment, in treated animals or patients; and, (iii) the ability of researchers and physicians to track and monitor the status, progress, and results of a treatment, and the locations and concentrations of exogenous genes and/or polypeptides.

This section describes various examples of such known techniques and sequences, along with a brief indication of how they can be applied to the methods and vectors of this invention. This listing of illustrative examples is not exhaustive or exclusive, and those skilled in the art will recognize various other genetic engineering techniques, reagents, and gene and peptide sequences and fragments that can also be adapted for use as disclosed herein.

In addition, it should be understood that the techniques and/or sequences disclosed herein can be combined with each other, in various ways that will be apparent to those skilled in the art; as just one example, a gene construct can be developed, for expressing a gene encoding for a mature neurotrophin in BBB-straddling neurons, which encodes a polypeptide that contains both: (i) a pre-pro-BDNF leader sequence, at the N-terminus of the sequence encoding the mature neurotrophin, and (ii) an “epitopic tag” placed elsewhere in the sequence encoding the mature neurotrophin. Similarly, it should be recognized that other, additional genetic engineering techniques or sequences that are now known or hereafter discovered may also be adapted for use as disclosed herein.

As one example of a technique for increasing the expression of a passenger polypeptide carried by a genetic vector, the polypeptide coding sequence can be placed under the control of a powerful “gene promoter” sequence that will drive high levels of transcription of mRNA strands containing the coding sequence. Various promoter sequences that act as strong promoters in human cells (including human neurons) are known, and include, for example, promoter sequences derived from various types of pathogenic viruses, such as a cytomegalovirus (CMV) promoter, a Rous sarcoma virus (RSV) long terminal repeat (LTR) promoter, and a simian virus 40 (SV-40) “early” promoter.

Alternately, in some cases, it may be preferable to place one or more genes (such as, for example, a gene which encodes a marker polypeptide) under the control of a so-called “inducible” promoter, which will be active only under certain conditions or when a certain compound is present.

Of potentially even greater interest herein than “inducible” promoters is a class of gene promoters that are usually referred to as “tissue-specific” promoters. These types of promoters cause a polypeptide coding sequence to be efficiently expressed into mRNA, only in specific types of tissues or cells. If a gene construct containing a tissue-specific promoter is delivered into the wrong cell or tissue type, those “non-intended” cells usually will not have the transcription factors or enzymes which can recognize that particular tissue-specific promoter; therefore, the gene construct will not be expressed (or will be expressed only at low rates) in those types of “non-intended” cells. Accordingly, tissue-specific promoters offer potentially very useful candidates for evaluation as described herein.

From published transgenic animal studies, a number of tissue-specific promoters have already been identified and published, and their number is increasing as additional studies on transgenic animals are published. The following non-exhaustive, non-exclusive list contains several examples of tissue-specific promoters that may be useful in this invention:

(i) tissue-specific promoters which appear to be much more active in olfactory receptor neurons than in other classes of neurons include an olfactory marker protein promoter described in Servenius et al 1994, and an M4 olfactory receptor protein promoter described in Qasba and Reed 1998;

(ii) a tissue-specific promoter which appears to be much more active in nocioceptive sensory neurons than in sympathetic neurons is part of the gene which expresses the “minimal calcitonin gene related peptide” (CGRP), as described in Watson et al 1995. This gene may also offer a chemically-inducible gene promoter as well, since it appears to be much more active in the presence of nerve growth factor (NGF). Therefore, if the CGRP gene promoter is used, gene expression in sympathetic neurons could be minimized, which probably would minimize undesired side effects. This type of gene promoter may be especially useful, since nocioceptive sensory neurons as well as sympathetic neurons are known to internalize NGF, via a process called “receptor-mediated endocytosis”. Since both nocioceptive and sympathetic neuronal types will likely be transfected, if peripheral administration is used with a non-viral vector that exploits NGF-receptor-mediated endocytosis to target gene delivery to accessible neurons, the ability to create a gene construct that will not be expressed at substantial levels inside transfected sympathetic neurons, by using a gene promoter such as the CGRP promoter, may be highly advantageous.

(iii) a tissue-specific promoter which appears to be much more active in spinal motor neurons than in nocioceptive or other sensory neurons drives expression of the alpha-1 subunit of glycine receptors, as described in review articles such as Bechade et al 1994, Rajendra et al 1997, and Zafra et al 1997.

In general, where a transgene construct has been shown to restrict expression of a selected gene to a particular class of neurons in a line of transgenic animals, the same pattern of restricted gene expression can be expected when an appropriately prepared gene construct (containing the same promoter and associated gene expression elements) is delivered in vivo by viral or non-viral vectors. Thus, reference should be made to transgenic animal protocols (e.g., Causing and Miller 2000) when designing gene expression constructs.

In a related and similar manner, it also may be possible and desirable to use naturally-occurring or synthetic promoters and/or enhancers to further stimulate, restrict, or control expression of a foreign gene in various ways. For example, it may be possible to establish or increase foreign gene expression in injured neurons, without substantially affecting other neurons, by using a gene promoter which appears to be specifically activated after a nerve lesion, but which appears to be otherwise silent (see Funakoshi et al 1998). Alternately, a glucocorticoid-responsive promoter may be used to drive gene expression after neurological insult when, as a result of the stress response, glucocorticoid-adrenal stress steroids are present in high levels (e.g., Ozawa et al 2000). Alternately, the latency-associated transcript promoter from herpes simplex viruses (Lachmann et al 1997) is likely to drive gene expression in a manner that may be more prolonged than can be achieved by various other promoters.

As another example of a technique that can be used to increase protein expression, scientists who create engineered genes for transfection often delete one or more “non-preferred” codons and replace them with “preferred” codons. The distinction between preferred vs. non-preferred codons arises from the fact that most species (and many different cell types, within a certain species) have evolved with a repertoire of both: (i) preferred codons, which will pass quickly and without delay through the translation system inside ribosomes, and (ii) non-preferred codons that will slow things down, inside ribosomes. This system of preferred and non-preferred codons provides a cellular mechanism that is highly useful for regulating gene expression, so that properly balanced quantities of thousands of different proteins can all be present at suitable concentrations, inside a single cell. However, in genetic engineering, by getting rid of non-preferred codons and replacing them with preferred codons, a genetic vector can bypass the normal control system, and drive the production of unusually high levels of a foreign protein. This type of approach is described in numerous published works, such as U.S. Pat. No. 5,795,737 (Seed et al, 1998) and various articles cited therein.

Another known genetic engineering trick involves “cysteine-depleted” variants of a polypeptide. As is well known, residues of cysteine (an amino acid with a highly reactive sulfhydryl group, —SH) in a polypeptide chain tend to react with other cysteine residues. When two cysteine residues react with each other, they form a disulfide bond. Disulfide bonds are very important in ensuring that, when a polypeptide is being synthesized inside a cell and is being subjected to “post-translation processing”, it will be folded into its proper three-dimensional configuration. However, when genetic engineering is used to create alternate processes, cysteine residues often become highly problematic, and stand in the way of the desired result. Therefore, such problems can often be avoided and overcome by replacing one or more cysteine residues, at certain locations in a polypeptide sequence, with other residues. This is done by replacing the codon which specifies a cysteine residue, at some particular location in a gene, with a different codon that will specify some other non-cysteine amino acid residue. These types of cysteine-depleted “muteins” are described in various U.S. Pat. No., such as 4,737,462.

Genetic engineering techniques also can be used to add a so-called “leader sequence” and/or “signal sequence” to a foreign polypeptide that is encoded by a genetic vector as disclosed herein. Although the term “leader sequence” is not always used consistently or precisely, it generally refers to a polypeptide sequence which has either or both of the following traits: (i) it causes or promotes the transport of the resulting leader-plus-polypeptide molecule to a certain location in a cell, or the secretion of the leader-plus-polypeptide by host cells, as can be demonstrated when the leader sequence is added to foreign polypeptides that normally do not undergo that type of transport or secretion; and/or, (ii) it is the portion of an initial polypeptide which is subsequently cleaved off, by natural “post-translational processing,” from a smaller version of the final, “mature”, or active polypeptide. In nearly all cases, a leader sequence will be positioned at the N-terminus of a polypeptide; that is the end (often called the “head”) which is created first, and which emerges first from the ribosome as the polypeptide is created.

Similarly, the term “signal sequence” is not always used consistently or precisely. In general, it refers to a polypeptide sequence that leads to some particular type of result, effect, or process, as can be demonstrated by the ability of that signal sequence to impart the same result or process to other peptide sequences that normally do not undergo that result or process. As examples, signal sequences can lead to (i) enclosure, sequestering, or other processing or “packaging” of a polypeptide into vesicles or other compartments; (ii) transport of a polypeptide to a particular component or region of a cell; or (iii) secretion of a polypeptide by the host cell. Accordingly, “signal sequence” is used more broadly than “leader sequence”, and generally includes leader sequences. In addition, there is no implication that a signal sequence must be cleaved off of an initial polypeptide, to create a mature final version of the polypeptide; signal sequences are often retained by fully mature polypeptides.

Various neuronal leader and/or signal sequences are known that are believed to increase either or both of two processes that are highly useful in this invention. Those two processes are: (i) anterograde transport of a polypeptide, out of the main body of a neuron and through a “central projection” which will transport the polypeptide, while still inside the cell where it was synthesized, across the blood-brain barrier and/or closer to a desired targeted area of the brain; and/or, (ii) secretion of a polypeptide, by a neuron, at synaptic or other terminals belonging to the neuron.

One such type of leader sequence which may be highly useful, in various settings, is a leader sequence that is initially found in a polypeptide called “pre-pro-BDNF”, where BDNF is the acronym for brain-derived neurotrophic factor. BDNF is a homologue of nerve growth factor (NGF), but unlike NGF, pre-pro-BDNF is known to be efficiently transported, in an anterograde direction, by nocioceptive neurons. This has been confirmed by animal tests, showing that BDNF that was expressed inside nocioceptive neurons can be detected later in spinal tissue. The shorter, final (or “mature”) form of BDNF is created when the leader sequence is cleaved off of the initial longer polypeptide. The BDNF polypeptide, and the pre-pro-BDNF leader sequence, are described in a number of published articles, including Conner et al 1998, Altar et al 1999, and Tonra 1999. Anyone skilled in this particular art who evaluates what is known about the BDNF polypeptide and the pre-pro-BDNF leader sequence, will recognize that this leader sequence, and any other peptide leader sequence which is known to enable or increase anterograde transport within neurons and/or secretion by neurons, may be highly useful in increasing the success rates and efficacy levels of this invention.

### EPITOPIC TAG SEQUENCES

Another genetic engineering technique also deserves attention, since it may be highly useful in this invention. This technique involves “epitope” sequences, tags, and constructs.

The prefix “epi-” refers to an exposed and accessible surface; as examples, the epidermis is the outermost dermal (skin) surface, and the epithelium is the exposed surface of a mucous membrane.

Similarly, the term “epitope” is used to refer to a surface-exposed domain of a protein which can be firmly bound by an antibody. An antibody does not wrap itself around the entire surface of an antigenic protein; instead, a localized binding domain which is part of the antibody will bind to a localized binding domain which is part of the antigen, in a manner that is analogous to two jigsaw pieces fitting together along one (and only one) edge of each piece.

By using tests in which digested (cleaved) fragments of an antigenic protein are passed through a column in which monoclonal antibodies have been affixed to tiny beads, it is not difficult to identify the epitopic-region of an antigenic protein, with respect to a particular line of monoclonal antibodies. However, different lines of monoclonal antibodies will bind to different surface areas of an antigenic protein; accordingly, a protein generally has a number of different epitopic regions, and the common factor that determines which regions are epitopic is whether a region is exposed, and accessible to antibodies, on the surface of the protein. For this reason, epitopic analysis is often used by researchers to help them determine the three-dimensional structure of a complex protein; as a general rule, the amino acid sequences which show up as epitopic sites in a protein are the amino acid sequences that are exposed, and accessible to antibodies, on the surface of the protein.

The practice of using epitopic “tag” sequences in genetic engineering arises from the following fact: it is relatively easy to create and identify monoclonal antibodies that will bind, with very high levels of selectivity, to known epitopic sequences. Several such monoclonal antibody lines (and the corresponding high-affinity antigenic amino acid sequences to which they bind) are well-known; examples include monoclonal antibody lines that will bind to known amino acid sequences derived from polypeptides such as c-myc, and a haemagglutinin protein from the influenza virus.

Because polypeptide expression in cells starts at the N (amino) terminus, and ends at the C (carboxy) terminus, there is a generally high probability that amino acid segments located at or near the C terminus will be exposed on the surface of the protein. This arises from the fact that the folding and shaping process which generates the final three-dimensional of the polypeptide will begin taking place as the strand is being created and extended, due to various amino acid residues or domains located along the strand in specific locations attracting or repelling each other. Although this depiction is highly simplified and does not adequately address the subtleties of protein folding, one can envision the first part of the strand that emerges from a ribosome as forming the “core” of the emerging polypeptide, and the rest of the strand generally being wrapped around or otherwise added to the initial core.

Because of this factor, amino acid sequences that are at or near the “tail end” (the carboxy terminus) of a polypeptide have a relatively high likelihood of being exposed and accessible, as epitopic sites, on the surface of the polypeptide. To take advantage of this, factor, and to reduce the risk that insertion of an epitopic site into a central domain might disrupt or degrade an essential activity or trait of the polypeptide, the normal practice is to position epitopic tag sequences at or near the tail end (the carboxy terminus) of a polypeptide that is being modified to include an epitopic tag. This generally offers the best approach to initial research using epitopic tags, and experimental data obtained using this approach can be used to optimize subsequent efforts, if necessary.

Alternately or additionally, if the full three-dimensional structure of a polypeptide molecule is known, and if the polypeptide is known to have an “active” site that is crucial for catalytic activity or receptor binding, it may be possible to insert a surface-accessible epitopic site into the polypeptide, on the opposite side of the molecule.

By using relatively simple in vitro screening tests, any such modified “tagged” protein can be tested to determine whether it still has the desired activity, and tagged proteins which pass those initial tests can be tested more extensively using in vivo animal models.

Accordingly, the genetic vector system disclosed herein allows a gene construct to be created, in which a unique or at least highly uncommon and detectable “epitopic tag sequence” can be added to (or inserted into) nearly any type of known protein (such as, for example, nerve growth factor). Using known genetic engineering techniques, it is a straightforward procedure to modify the coding sequence of a gene construct carried by a genetic vector, in a manner which will add a relatively small number of additional codons to the “native” coding sequence, or which will substitute and replace a few codons in the native coding sequence by other codons that do not normally appear in the native protein.

In various forms of research and treatment that are taught herein, genetic vectors carrying gene constructs that will express epitope-tagged polypeptides can be highly useful, since they can make it much easier (and in some cases, they may be required to make it possible) for researchers and physicians to monitor and quantify the results and effects of the genetic treatments disclosed herein. As one example of the types of problems that can be encountered when naturally-occurring polypeptides must be analyzed, under the current state of the art, naturally-occurring nerve growth factor (NGF) tends to be degraded by most types of tissue fixation methods, it is very difficult for even highly skilled researchers to measure it accurately, and frustratingly high degrees of homology and antibody cross-reactivity exist in the different versions of NGF that are found in animals as different as rats and humans. Epitope-tagged versions of NGF (and of various other neuroactive polypeptides) offer promising methods of avoiding and overcoming those types of difficulties.

It also should be noted that epitope-tagged polypeptides are used much more frequently and widely in research, than in clinical practice on humans. In general, epitope-tagged polypeptides are used most commonly for developing, optimizing, and proving the effects and efficacy of a certain treatment approach, in cell culture tests and animal tests. In addition, they sometimes are used in small-scale “Phase 1” or “Phase 2” human clinical trials, which are authorized under “Investigational New Drug” applications in the U.S., and under similar governmental review and approval mechanisms elsewhere. By the time a treatment is ready to be tested in larger multi-site Phase 3 clinical trials, any epitopic or other “foreign” sequences usually will be removed, to reduce the risk of generating an immune response involving antibodies that would recognize and attack the epitopic sequence.

### REPEATED CYCLIC LIGAND SELECTION METHOD

This patent application also discloses what appears to be a novel approach to using a cyclic/repeating series of steps to identify, isolate, and develop effective polypeptide ligands that can drive active and selective endocytosis, to take advantage of receptor-mediated endocytotic receptors on targeted neurons. This cyclic ligand method is regarded as a separate and distinct invention, since it has a number of potential uses that extend beyond use as disclosed herein; however, since it appears to be very well suited to providing new and enhanced ligands that can selectively drive the process of neuronal endocytosis, which is one method for carrying out the invention disclosed herein, this cyclic ligand method is briefly summarized herein. A more extensive description is contained in an Australian provisional patent application, number PS1935, which was filed in April 2002. A copy of that Australian provisional application is being submitted along with this patent application, and that copy of the provisional application will become open for public inspection and copying upon issuance (and possibly upon earlier publication) of this patent application.

This approach takes advantage of two key facts, which can be summarized as follows. First, various types of cells (including neurons) can internalize phage particles, if the phage particles can attach and bind in a reasonably tight manner to “endocytotic receptors” on the surfaces of the cells. As noted above and as described elsewhere, eukaryotic cells have evolved with various types of cell surface receptors that, when bound by a “ligand” molecule, will commence a series of steps that lead to internalization of the ligand-receptor complex (i.e., the receptor protein, plus the ligand molecule which has become temporarily attached to that receptor). This type of internalization process most commonly involves the creation of a “lipid vesicle” which surrounds the ligand-receptor complex, and which is made of the same type of lipid bilayer that forms the cell's outer membrane. These lipid vesicles can be large enough to enclose entire phage particles (e.g., Hart et al 1994; Becerril et al 1999).

The term “phage” is used to describe certain types of viruses that will attack and kill E. coli, the well-known bacterial “workhorse” of genetic engineering. The entire genomes of various strains of phages have been completely sequenced, and many also have been manipulated to create “restriction sites” at unique and useful locations. These restriction sites allow a strand of DNA to be opened up at a unique location, by a type of enzyme called a “restriction endonuclease”. This allows insertion of a foreign DNA sequence into a location in the phage DNA which will cause a polypeptide sequence encoded by the foreign DNA to appear in an exposed location on the surface of a phage's capsid proteins.

In addition, some types of phages have been genetically engineered to insert an “origin of replication” into the phage. This DNA sequence allows the phage DNA to be reproduced, in selected types of host cells, in a manner directly comparable to a plasmid being replicated inside that type of host cell. These types of phages are often referred to as “phagemid”, which is a hybrid name derived from phage and plasmid. As used herein, phagemids are regarded as a subset of phage, and any references to phages include phagemids as well.

For the reasons discussed above, phages are highly useful for various types of genetic engineering work and research, since they allow each of the following steps to be carried out in a relatively cheap, simple, and rapid but fully controllable manner: (i) replication of phage DNA containing foreign sequences that have been inserted into controlled locations in the phage DNA; (ii) creation of abundant copies of proteins containing polypeptide sequences encoded by the inserted DNA sequences; (iii) the use of “shotgun cloning” and various other techniques (described in articles such as Winter et al 1994, Schatz 1994, etc.) to create “phage libraries”, which can contain thousands of randomly generated, selected, enriched, or other random or controllable mixtures of different DNA sequences and/or polypeptide sequences, inserted at known and controlled locations into phage genomes that can then be manipulated easily; and (iv) the use of assay procedures to screen phage libraries, to identify and isolate particular phages that happen to express a polypeptide sequence that is of interest in some particular procedure or test.

The second fact which is used and exploited, by the cyclic ligand selection process disclosed herein, involves a matter of cell physiology in neurons. As discussed above, in neurons that straddle the BBB, the long cellular fiber that crosses the BBB is called an “axon” of that neuron. In a surgical technique that is usually referred to as ligation, or emplacing a “ligature”, the flow of cellular fluids through that axon can be choked off and blocked, by placing a loop of suture material around the axon at that location, and then tightening the loop. This is most commonly done by placing a ligature loop around an entire nerve bundle, rather than around just one neuronal axon.

It should be noted, in passing, that the terminology involved can be confusing. When used in the surgical manner, as briefly described above, “ligate” refers to a procedure for dividing a neuron into two separate domains, without actually cutting the axon, by tightening a suture strand around the axon. In an entirely different meaning, used in biochemistry and genetic engineering, “ligate” refers to connecting two different strands of DNA together, to form a single DNA strand (or connecting two different ends of the same strand to each other, to form a closed loop). This is normally done by using an enzyme called DNA ligase. Since those two meanings for ligation are entirely different from each other, they should not be confused.

Also, it should be noted that both of these terms are entirely different from yet another similar term, “ligand”, which refers to a molecule that will react and bind with specific affinity to another molecule, such as a receptor or antibody.

Surgical ligation of neuronal axons is accomplished most commonly by placing a ligature strand around a sciatic nerve bundle, in or near the hip or hind leg of a rat. The term “sciatic” was derived from the Greek word referring to “hip”, and each sciatic nerve passes through the hip structure and innervates one of the animal's hind legs. This nerve bundle is still fairly large in the hip and upper leg, before it divides into smaller branches farther down the leg, and it can be accessed without difficulty during a relatively minor surgical procedure. Procedures for ligating neurons (and for injecting phage into sites in a way which will maximize contact with axon termini) in this type of work can be adapted from methods previously used to study selective internalization and retrograde transport of radiolabelled neurotrophic factors in other contexts; such procedures are described in articles such as Ferguson et al 1990 and 1991.

If a ligature is placed around a sciatic nerve bundle and then tightened, the flow of intracellular fluids through the sciatic axons will be choked off and stopped, at the location of the ligature. Within a span of roughly 24 to 36 hours, the “distal” portions of the axons (the portions between the ligature, and the foot) will begin to die, and will eventually disintegrate and dissolve, leaving the hind leg permanently paralyzed.

The part of an axon located between a ligature and an animal foot is sometimes referred to as being “downstream” of the ligature, since most fluid transport in an axon travels away from the main cell body, toward the tip of the axon (i.e., toward the animal's foot). However, the term “downstream” can be confusing when used in the manner of interest herein, since the tip of the axon, in the animal's foot, will be upstream of the ligature, with regard to molecules undergoing retrograde transport. To avoid confusion, the portion of an axon that is being starved and killed by a tightened ligature is referred to herein as the distal portion, rather than using terms such as downstream or upstream.

The process of death and necrosis in the distal portion of a ligated axon does not occur immediately, and a variety of cellular processes inside the distal axon will continue for a period of roughly a day after a ligature is emplaced and tightened. In particular, if a ligand molecule binds to and activates an endocytotic receptor at or near the tip of a ligated sciatic axon, shortly before or after the ligature is tightened, endocytosis of the ligand-receptor complex will still occur, and it will pull the receptor-ligand complex into the cytoplasmic fluid inside the neuron. Once that has been accomplished, retrograde transport of the ligand-receptor complex will commence, and will continue for a period of roughly 18 hours, inside the distal axon. However, the internalized ligand-receptor complex will not be able to pass through the “chokepoint” where the ligature crimps and squeezes the axon. Therefore, any ligand-receptor complexes which have been taken into distal axon, via receptor-mediated endocytosis, will be effectively trapped inside the distal axon fiber.

If the facts summarized above are placed next to each other, the logic and reasoning behind the cyclic use of phage libraries to identify and select ligand polypeptide molecules that can trigger and drive the process of receptor-mediated endocytosis will begin to become clear. A phage (or phagemid) library can be constructed, with hundreds or thousands of phages, wherein each phage contains a single polypeptide sequence which is exposed on the surface of that particular phage. A liquid solution containing this “library” of phages is then injected into the foot or hindleg of a rat, in a location that will cause the injected phages to contact the termini of sciatic nerve axons, in the leg or foot of the animal. This injection is carried out shortly before or after a ligature is tightened around that sciatic nerve bundle. The phage particles which happen to display polypeptide sequences that will bind to endocytotic receptors, on the surfaces of the sciatic axons, will be taken inside the axons by the process of endocytosis, and will then be retrogradely transported toward the main cell body of the neuron. However, the process of retrograde transport will not be able to carry the ligand-receptor complex(es) past the tightened ligature, located near the animal's hip. Therefore, the ligand-receptor complex(es) that have been taken inside a sciatic axon, by receptor-mediated endocytosis, will be trapped inside the distal portion of the axon.

At an appropriate time (such as roughly 18 hours after the ligature loop was tightened around the sciatic axon bundle), before the process of necrosis and disintegration begins to destroy the distal portions of the axons, the rat is sacrificed, and segments of the axons distal to the ligature point (generally including, for example, a range of about 1 to about 3 mm beyond the ligature point) are surgically harvested, and separated to at least some extent from surrounding cells and tissues that are not of interest. Since the harvesting region can be relatively distant from the injection site, the risk of contamination, by undesired phages that (for example) might have become bound to non-internalized molecules on the surfaces of neurons and/or target tissues, can be minimized.

The harvested axonal segments can then be processed in a way that will break part the axons without destroying viable phage particles contained in the axons. This can be done by suspending the nerve segments in a liquid, and processing the liquid and cell fragments briefly in a suitable device, such as a tissue homogenizer or possibly a sonicator.

The resulting crude suspension will contain phages that were located inside the sciatic nerve axons. Since entry of phages into neuronal axons occurs at a substantial rate only through the process of receptor-mediated endocytosis, this creates the presumption that those phages which are present in a homogenized liquid suspension as described above will be phage particles that happened to display polypeptide sequences that reacted with endocytotic receptors on the axons, in the foot or leg where the phage library was injected. Those phage particles, in viable form, can then be used to infect E. coli cells, where they will reproduce into another library that will contain multiple different phages, each displaying a certain type of polypeptide sequence. This library will be “enriched” for phages which display polypeptide sequences that will bind to endocytotic receptors on the accessible peripheral projections of sciatic neurons.

This enriched phage library can be carried through the same selection process again, and that same cycle can be repeated as many times as desired. At any given point during that type of cyclic screening and selection process, a clonal colony of phages can be isolated from the library, and grown in E. coli cells. That clonal line can then be analyzed, to determine the genetic sequence of the DNA insert that is being expressed in the coat protein of that particular phage line. This DNA sequence will reveal the exact amino acid sequence of the polypeptide fragment that was binding to endocytotic receptors on the sciatic neurons.

That revealed polypeptide sequence (and/or a random or controlled mutant or variant thereof, which appears in high quantities when tested in the same cyclic screening process) can then be used as a polypeptide ligand sequence, which will bind with relatively high affinity to endocytotic receptors on the peripheral projections of at least some types of neuronal axons. As such, that polypeptide ligand sequence can be incorporated into one or more types of viral and/or non-viral genetic vectors, thereby causing those enhanced genetic vectors to be taken up, into BBB-straddling neurons, at higher rates.

Accordingly, it is believed and anticipated that the types of polypeptide ligand sequences which can be identified, isolated, and analyzed through this type of cyclic screening process, can enable researchers and physicians to create substantially enhanced genetic vectors, which will be taken up into BBB-straddling neurons at increased rates, when compared to other vectors which do not use this approach. It is also believed and anticipated that this approach may be able to enable the creation and refinement of various types of enhanced non-viral vectors that will be able to accomplish highly useful results that cannot be achieved by any non-viral vectors available today.

In another potentially useful offshoot of this work, it also appears likely that a subpopulation of internalizable ligands selected by this approach may be able to enable delivery of functioning genes (rather than just polypeptides) to neurons that reside wholly within the blood-brain barrier. This might be accomplished if a selected ligand can bind reversibly to receptors which mediate trans-synaptic transport of various molecules. Various such trans-synaptic transport molecules are known, including tetanus toxin (Price (1975; Schwab et al 1979) and certain lectins, such as barley lectin (Horowitz et al 1999) and wheat germ agglutin (Yoshihara et al 1999).

### USING GENETIC VECTORS FOR DRUG ADMINISTRATION/DELIVERY INSIDE BBB

The invention may be used by administering, into or onto tissue which is not protected by the BBB, one or more copies of a gene vector that can transfect one or more types of BBB-straddling neurons. Clearly, the range of preferred and potential modes of administration will depend on the type of BBB-straddling neuron that is to be transfected by the vector, and the type and location of the peripheral projection that is to be contacted by the vector. As described above and in the examples, administration to olfactory receptor sensory neurons can be via nasal instillation; administration to nocioceptive neurons can be via cutaneous, subcutaneous, or possibly intramuscular injection, and possibly by topical administration (which can be accompanied by one or more agents or techniques that will increase epidermal penetration and tissue permeation, as described above); administration to lower motor neurons innervating the skeletal musculature can be via intramuscular injection; and, administration to the lower motor neurons of hypoglossal nucleus can be by injection into the muscles of the tongue.

It will be apparent to those skilled in the arts of medicine or neuroscience that there exist other ways to administer gene vectors to peripherally-projecting neurons. It also will be apparent to such persons that: (i) other types of neurons that straddle the BBB (including various types of sensory and motor neurons, as well as pre-ganglionic neurons of the sympathetic and parasympathetic systems) offer potentially useful targets for transfection; and, (ii) the known features and traits of the anatomy and structure of any such class of neuron will allow skilled neurologists and researchers to develop various methods for administering gene vectors to such neurons.

### MODULATION OF ENDOCRINE AND PARACRINE HORMONAL SYSTEMS

It should also be recognized that, by enabling non-invasive delivery of specific gene-encoded polypeptides to cells, systems, and regions within the brain, this invention may also be able to provide new and previously unavailable methods and approaches to controlling or modulating various types of “downstream” effects or activities, such as by increasing or suppressing the release of various types of endocrine and/or paracrine hormones by various glands or organs, either in the CNS-protected brain tissue (such as the pituitary and pineal glands), or in other parts of the body (such as the thyroid, thymus, adrenal, or other glands, or in the pancreas, reproductive organs, etc.).

This patent application is not the appropriate location for a detailed analysis of the endocrine or paracrine systems, and the comments below are intended solely as a very brief introduction and overview. For more information on the endocrine and paracrine systems, good overviews are provided in nearly any good textbook on physiology, and more information is contained in numerous full-length textbooks, such as Wilson & Foster 1992, Barrow & Sleman 1992, Brown 1993, DeGroot 1994, etc. Recent review articles are not especially helpful,in establishing a working knowledge of the endocrine or paracrine systems, since they focus mainly on problems (such as glandular tumors, hormone disruptors such as pesticides, etc.), interventions (surgical or drug), or interactions between hormone systems and other systems such as immune responses; however, review articles offer a good base of information when the goal is to move beyond a working knowledge of the endocrine or paracrine systems, and into the realm of potential interventions for purposes such as therapy of human disorders or malformations, or livestock breeding.

One of the crucial components of the endocrine system is the pituitary gland, which sits at the base of the brain, suspended from a region of brain tissue called the hypothalamus. The anterior lobe or gland of the pituitary is known to release at least six different hormones, and the release of each of these hormones is either triggered or suppressed by an “upstream” hormone, called a hypothalamic hormone. These hormonal systems (or pairings, relationships, etc.) include the following:

1. a hypothalamic hormone called thyrotropin-releasing hormone (abbreviated as TRH; formerly called thyroid-stimulating hormone releasing hormone) causes the pituitary to release a hormone called thyrotropin (formerly called thyroid-stimulating hormone, or TSH);

2. a hypothalamic hormone called corticotropin-releasing hormone (CRH) causes the pituitary to release adrenocorticotropin;

3. a hypothalamic hormone called growth hormone releasing hormone (GHRH) causes the pituitary to release growth hormone (also called somatotropin, and often referred to as hGH or HGH in the case of human growth hormone);

4. a hypothalamic hormone called growth hormone inhibitory hormone (GHIH, also called somatostatin) inhibits the release, by the pituitary, of growth hormone (somatotropin);

5. a hypothalamic hormone called gonadotropin releasing hormone (GRH or GnRH) causes the pituitary to release two types of “gonadotropic” hormones, called luteinizing hormone, and follicle-stimulating hormone; and,

6. a hypothalamic hormone called prolactin inhibitory hormone (PIH) inhibits the release, by the pituitary, of a hormone called prolactin.

By administering a genetic vector that will cause an increase in the concentration of one of the above-listed hypothalamic hormones inside BBB-protected brain tissue (it should be noted that a transient rather than permanent increase can be achieved by the methods disclosed herein, and transient increases are generally presumed to be preferable in therapeutic treatments of human medical or developmental disorders), it is likely to be possible to cause, in a controllable manner, either stimulation or inhibition of the release of a “downstream” or “dependent” pituitary hormone. Accordingly, as a result of that type of triggered and/or targeted pituitary stimulation or inhibition via a genetic vector, it is possible to stimulate, inhibit, or otherwise modulate the same types of physiological effects that are caused by the release, or inhibition, of the pituitary hormones.

As an alternate approach, it may be possible in at least some cases to stimulate or inhibit a targeted endocrine or paracrine system by administering a genetic vector that directly encodes a pituitary hormone, rather than its “upstream” hypothalamic hormone. Along these lines, radiolabelled tracer studies have shown that at least some types of proteins which have been delivered into the CNS by direction injection into a brain ventricle are cleared fairly rapidly from the CNS into the blood circulation (e.g., Ferguson 1991). Therefore, if the rate of delivery of a hormone-type polypeptide into BBB-protected CNS tissue is sufficiently high, some of those hormone polypeptide molecules will diffuse into circulating blood, and will be distributed systemically.

With regard to delivering hormonal or other polypeptides into the brain with the intent of causing the polypeptides to contact specific regions, cells, or structures within the brain, it should be borne in mind that this invention may be able to offer, in at least some cases, an approach which will allow targeted delivery in ways that have not previously been available. Most prior art methods of delivering neuroactive molecules (such as neurotrophic factors) into the CNS appear to assume that the endocrine model of drug delivery is the appropriate method for delivering such molecules. Evidence that this assumption is prevalent is seen in numerous animal studies, as well as limited human clinical studies in which recombinant neurotrophins are injected or infused into cerebrospinal fluid (typically into the lateral ventricles). These drug administration approaches are based on the assumption that the flow of cerebrospinal fluid within the brain and spinal cord represents an internal CNS circulation, analogous to the circulation of blood within the periphery.

However, that assumption is unwarranted, because the flow of CSF in the CNS is more analogous to the lymphatic system, in the periphery, than to blood circulation. Like the flow of lymph fluid, the flow of CSF is more of a uni-directional drainage, rather than a re-circulation of fluid.

Accordingly, when neuroactive molecules (such as neurotrophic factors) exert their physiological effect, not in an endocrine (system-wide) manner but rather in a paracrine (localized) manner, an appropriate drug delivery method preferably should not involve systemic administration, such as by intravenous infusion or injection, both because of (i) high levels of wastage of highly expensive drug compounds, and (ii) the potential for unwanted adverse effects, when systemically-injected molecules react with various cells or organs other than the desired targets. Instead, a more narrowly focused and targeted system should be used, if available.

This invention appears to offer a substantially improved method of drug delivery, which in some respects emulates paracrine delivery. This method can achieve or at least promote localized and focused delivery of drug to a target cell or region (especially when compared to other methods, such as intravenous injection) by transfecting only certain selected populations of BBB-straddling neuron(s), which will subsequently release polypeptides in a limited and desired “secretion zone”.

The ability to use gene therapy to achieve sustained drug delivery has been recognized in the art, as evidenced by numerous studies involving transplant into the CNS of cells that have been genetically engineered to secrete particular recombinant molecules, such as neurotrophins. However, nearly all such studies illustrate or imply that the desired intention is to achieve an endocrine-like form of drug delivery, with the transplanted cells secreting drugs in a manner that causes or allows systemic distribution. By contrast, this invention discloses an entirely different form of genetic therapy, which can achieve a paracrine-like, localized delivery of therapeutic polypeptides to a specific cluster and/or type of neuron within the BBB, by transfecting a limited number of neighboring cells that straddle the BBB.

### SELECTIVE MODULATION OF NEURONAL SYSTEMS

The invention enables development of completely new approaches to treating disorders of the nervous system, and the disclosed paracrine-like method of drug delivery enables development of new processes or methods for selectively modulating the function of particular systems of neurons within the CNS.

Physiologically, the function of systems of neurons within the central nervous system can change, in response to functional changes in the neurons that penetrate through the BBB. For example, changes in the electrical activity of nocioceptive neurons that penetrate through the BBB cause changes in the electrical activity of second order sensory neurons and the associated system of CNS neurons involved in sensation of pain and response to that pain. The anatomical connections between the neurons that project through the BBB, and neurons that reside wholly within the BBB, are not fixed and static; instead, the nature, number, and distribution of these connections can change, both over time and in response to various types of events (and often resulting in downstream changes in still other systems of neurons within the CNS). In other words, the systems of neurons within the CNS are plastic, changeable, and responsive to functional changes in the neurons that penetrate through the blood brain barrier.

Based upon that physiological fact, it is believed that in at least some cases, this invention may render possible to selectively modulate and alter the structure and functioning of at least some types of neuronal systems within the CNS, by altering the patterns of innervation (including strength, number, and distribution) of synaptic connections between transfectable peripherally-projecting systems, and targeted neuronal systems within the CNS.

As an example, it is well known in the field that neurotrophic factors are intimately involved in the plastic changes in synaptic density and innervation pattern, in CNS systems, in response to changes in electrical activity, such as the number and frequency of nerve impulses that arrive from other neurons located closer to the periphery. Physiologically, these neurotrophic factors act in what has been described above as a paracrine-like manner. It follows that the paracrine drug delivery approach disclosed herein can be used to selectively modulate the nature and extent of connections between one or more peripherally projecting neurons, and the CNS neurons or neuron systems with which they interact. That is, by using gene vectors to transfect BBB-straddling neurons, it will be possible (in at least some cases) to alter the nature and extent of the connections between those BBB-straddling neurons, and CNS neurons lying wholly within the BBB, and thereby selectively modulating one or more densities, functions, or other traits of the interacting CNS neuronal systems.

As examples, this principle can be illustrated by two preferred embodiments: (i) administration of recombinant anti-NGF, via transfection of nociceptive neurons that straddle the BBB, can modulate the BBB-protected neuronal systems that are involved in the perception of pain; and, (ii) administration of recombinant NT-3 or GDNF, via transfected BBB-straddling spinal motor neurons, can modulate the CNS neuronal systems involved in voluntary control over motor function.

### TREATMENTS FOR NEUROLOGICAL DISORDERS

This invention is likely to become useful for treating neurodegenerative disorders of the CNS (such as Alzheimer's disease), by methods which include delivering neurotrophic or neuroprotective factors to the neurons at risk of degenerating. It is believed, for example, that Alzheimer's disease probably can be treated in a useful manner by administering neurotrophic factors (such as nerve growth factor) into the CNS. In accord with that goal, this invention allows neurotrophic factors to be delivered, in a relatively focused and targeted manner, to basal forebrain cholinergic neurons that are at risk of degenerating in patients with Alzheimer's disease. In at least some patients, this type of treatment may be able to help slow, and potentially even halt, the neurodegenerative process.

This invention is also likely to become useful for treating trauma or injury to the CNS (such as that which occurs in head injury), by delivering neuronal growth factors (such as neurotrophic factors) to injured and surviving neurons. As just one example, GDNF, which acts on cortical motor neurons (which are frequently damaged in stroke or sometimes head trauma), can be delivered to these neurons by methods disclosed herein. Its potential benefits are described in various articles such as Schacht et al 1996.

This invention may also become useful for treating some cases of learning or memory dysfunction, such as occur in aging, dementia, after brain trauma or injury, and after various types of major surgery, especially surgery involving a cardiopulmonary bypass machine. Such trauma and insults often lead to loss of function within one or more regions of the CNS. Restoration of function, if it can be achieved, apparently requires and involves compensatory changes in the organization of the CNS, typically including the sprouting and outgrowth of various affected neurons, and the establishment of functional synapses on other neurons. These types of “neuroplastic” processes have been demonstrated both in animal studies, where a digit or limb was severed, and in human cases, where a stroke victim regained substantial use of a paralyzed limb by undergoing therapy in which one or two healthy limbs were strapped to the body and immobilized, while the patient did repetitive exercises which actively and aggressively challenged the patient to begin moving and using the impaired limb once again, building upon and expanding the range of motion and control that remained in the limb after the stroke.

Neurotrophic factors can play important roles in such processes (e.g., Lo 1995), and administration of NGF into the brain has been shown to enhance various CNS activities and functions, such as memory and learning (e.g., Fischer et al 1987 and 1991).

Accordingly, this invention provides a major avenue for expanding and enlarging upon that type of highly useful therapy, by allowing NGF and other neurotrophic and neurostimulatory factors to be delivered through the CNS and into the brain, using improved delivery systems.

This invention is also likely to become useful for treating disorders due to excitotoxic damage of neurons, or resulting from diseases or injuries that involve ischemia (inadequate blood flow, as occurs during a stroke or cardiac arrest) or hypoxia (inadequate oxygen supply, as occurs during drowning, carbon monoxide poisoning, etc.) or traumatic head injury. In animal studies, NGF infusion can slow or reverse the retrograde atrophy of cholinergic cell bodies and fiber networks and other changes in the cholinergic system that are caused by infarction or measured by infarct volumes or severity (e.g., Cuello et al 1992). Administration of NGF (or induction of NGF synthesis in vivo by clenbuterol) has been shown to reduce infarct volume in rat models of permanent middle cerebral artery occlusion (Semkova et al 1999). Other animal data suggests that NGF is able to act after a brain insult to block progression of neuronal damage (e.g., Guegan et al 1998). Since this invention is likely to prove useful for administering NGF after a stroke or other brain injury or insult, it likely will be able to reduce the extent and severity of subsequent neuronal loss.

The present invention further relates to methods for treating disorders of sensory function by modulating the function of the sensory neuron and/or the nerve cells which make synaptic contact with it in the CNS. For example, severe and persistent pain involves both nocioceptive neurons and CNS (spinal or brain) changes, and it has been reported that intrathecal administration of anti-NGF can reverse these types of changes, and alleviate the pain (e.g., Christensen et al 1996 and 1997). This invention allows the function of the sensory system to be modulated by delivering polypeptides (such as antibodies which will bind to and inactivate NGF, or which will occupy and block NGF receptors) which act on one or more components of the sensory system, to alter its physiological function.

This invention can also be used for modulating neuronal physiology by delivery and expression of neuropeptide genes, such as genes that express polypeptides that can block and suppress pain (such as so-called “endorphins”); genes that express growth factors; genes that express polypeptides to promote regeneration or prolong the life-spans of cells; and genes that express toxic polypeptides, such as to kill tumor cells.

Beyond that, this invention provides an approach that can be adapted to treatment of various types of CNS-related neurological disorders or deficiencies which are correlated with either too little or too much of some particular polypeptide. This can be accomplished by using this method to deliver, into BBB-protected CNS tissue, either: (i) a polypeptide which provides an additional quantity of a polypeptide, to reduce or eliminate a deficiency; or, (ii) a polypeptide which blocks, antagonizes, or otherwise suppresses a certain molecule, receptor, or reaction, thereby helping to controlling a CNS disorder that is caused or characterized by too much of a particular molecule.

### EXAMPLES

The Examples below are organized as follows:

Examples 1-8 relate to delivering neuron-stimulating polypeptides to neurons which lie wholly within the BBB, by transfecting olfactory receptor neurons, using vectors that carry genes which encode such polypeptides. For purposes of illustration, human NGF is used as the prototypic polypeptide; as will be recognized by those skilled in the art, genes which encode other forms of NGF (such as mouse, other rodent, or simian NGF, or any mutated, epitope-tagged, fragmented, or other form of NGF which may be of interest in medicine or research) may alternately be used.

Examples 1-7 describe how NGF (or other neurotrophic or similar polypeptides) can be delivered to cholinergic neurons in the basal forebrain of a laboratory animal, such as a rat. Examples 1-4 contain a complete embodiment, divided into various sequential steps. Example 1 describes the assembly of the vectors; Example 2 describes administration of those vectors to the nasal sinuses; Example 3 describes methods of monitoring delivery of the polypeptide through the blood brain barrier; and Example 4 describes methods of measuring the physiological and behavioral effects of such treatments on lab animals.

Following that “start to finish” description, Examples 5-8 describe delivery of an NGF-encoding or similar genes into olfactory receptor neurons, using different types of vectors. Example 5 describes vectors derived from herpes viruses; Example 6 describes liposome vectors; Example 7 describes vectors with ligands that bind to endocytotic receptors on neuron surfaces; and Example 8 describes vectors that use “transneuronal” polypeptides that can promote transport of the entire vector from one neuron to another.

Examples 9-13 involve a different approach, using gene vectors that encode nerve suppressing (rather than stimulating) factors; a polypeptide called “anti-NGF”, which binds to and inactivates NGF, is used as illustration. These vectors are injected into skin or muscle regions, in order to transfect nocioceptive (pain-signalling) neurons, in areas that suffer from unwanted and excessive pain signals (often called neuropathic pain, or allodynia). By suppressing over-active pain signalling circuits, this approach can help reduce and control neuropathic pain.

Examples 14-20 describe a third major line of approach, in which genetic vectors carrying genes that encode nerve-stimulating factors are injected into muscle tissue that is impaired due to a stroke, spinal injury, etc. These types of impaired muscles often suffer from a lack of (or impairments in) voluntary control, caused or aggravated by a loss of properly functioning connections between upper motor neurons (which lie wholly within the BBB) and lower motor neurons (which straddle the BBB). Transfection of lower motor neurons in such impaired muscles, using genes that encode nerve-stimulating factors, can help expand, repair, and reconnect the damaged motor control networks, by means such as establishing new and additional connections between the lower and upper motor neurons, and/or by increasing synaptic activity levels between those classes of neurons, thereby reestablishing proper innervation and CNS control over such muscle systems.

Example 21 describes transfection of certain types of motor neurons located inside the tongue. This route of administration deserves special attention, since it offers a route for delivering polypeptides into certain portions of the brainstem.

### Example 1

### Construction of Adenoviral Vector for Transfecting Olfactory Receptors with NGF Gene, to Deliver NGF to Cholinergic Neurons in the Basal Forebrain

Methods for preparing non-pathogenic vectors derived from adenoviruses that cannot replicate, except in genetically engineered host cells that exist only in laboratories, have been published in articles such as Graham and Prevec 1995. Methods for creating gene constructs which are small enough to be carried and delivered by adenoviral vectors, and which will express NGF (or some other CNS-active polypeptide, such as GDNF, NT-3, CNTF, or BDNF, or any of numerous other polypeptides such as listed in Table 1) at significant levels, inside transfected human neurons, are described in articles such as Romero et al 2000, Baumgartner and Shine 1998), Dijkhuizen et al 1997, and Gravel et al 1997. Methods for propagating, purifying, concentrating, and titering adenoviral vectors carrying such gene constructs can be found in publications such as the chapter by Engelhardt (pp. 169-184) in Methods in Molecular Medicine: Gene Therapy Protocols (P. Robbins, ed., 1997).

The gene construct that will drive expression of the NGF polypeptide (or other selected CNS-active polypeptide) will require proper selection of all relevant portions of the gene. As used herein, terms such as “gene” or “gene construct” normally refer to a complete and functional transcription unit, which can be manipulated under laboratory conditions using known procedures, and which, if transfected into a BBB-straddling neuron, is capable of: (i) causing the normal transcription and translation mechanisms inside the neuron cell body to synthesize the polypeptide encoded by the gene, and (ii) instructing the cell to appropriately process and secrete the mature polypeptide from dendrites, processes, synapses, or other terminals located inside the BBB.

A number of variants and enhancements of the simple “plain vanilla” type of gene constructs are known to those skilled in the art, including a number of variants and enhancements described in the Detailed Description section. Any such variants or enhancements are included within the terms “gene” or “gene construct” as used herein.

The complete amino acid encoding sequences (without introns) of human NGF, mouse NGF, and certain other forms of NGF, have been published and are known and available, in easily manipulated plasmid form, from various researchers; alternately, they can be created using well-known techniques and published information.

Also, if a polypeptide having an unusual “epitopic tag” sequence (such as c-myc) is desired, then the coding portion of the gene construct must include the DNA sequence that will encode the “epitopic tag” sequence. The inclusion of an epitopic tag may assist in demonstrating, refining, and otherwise enhancing the invention, in situations where epitopic tag sequences can assist researchers to distinguish between exogenous polypeptide molecules that are encoded by a genetic vector, and endogenous (native) polypeptides that are present naturally within the BBB of test animals or patients.

The other crucially important part of a gene construct is the gene promoter, and numerous known gene promoters are known and available which can drive gene expression in olfactory receptor neurons. Examples of “selective” gene promoters that are known to drive gene expression specifically in olfactory receptor neurons include promoters identified in the olfactory marker protein gene (Servenius et al 1994) and the M4 olfactory receptor gene (Qasba and Reed 1998). Use of such selective promoters can help minimize the chance that undesired or uncontrolled expression of the foreign gene will occur in surrounding cells or tissue.

Alternately, various types of viral and other promoters which are known to be unusually strong promoters in mammalian cells can be used if desired, for purposes such as inducing the highest practical levels of expression of NGF (or other CNS-active) polypeptide in transfected cells. Examples of such strong promoters include the early gene promoter from cytomegalovirus, and the late gene promoter from simian virus-40. Inducible gene promoters can also be used if desired, so long as the inducing factor which activates the selected promoter can be administered in a way which ensures that it will be transported into transfected neurons in adequate quantities.

As noted above, for simplicity of discussion herein, the term “gene promoter” as used herein includes the so-called “TATA box”, and the sequence of about 25 bases between the TATA box and the actual start of transcription.

Once the promoter and coding sequence of the gene construct have been chosen, various other portions of the gene construct can be chosen to enhance expression, packaging, transport, secretion, and/or performance of the vector-encoded polypeptide. These gene portions can include, for example: (i) a DNA sequence which will encode a “leader” or “signal” peptide sequence, which will instruct the transfected neuron to appropriately process and secrete the vector-encoded polypeptide (or a “mature” form from which the leader or signal sequence may be removed); (ii) a DNA sequence that will be transcribed into an mRNA sequence that will serve as a non-translated “tail” region, which will follow the “stop” codon in the mRNA, to facilitate translation and release by ribosomes; and, (iii) a transcription terminator sequence, which will direct RNA polymerase to truncate the mRNA strand when the DNA sequence is being transcribed into mRNA. These types of functional sequences are well-known, and can be obtained or adapted from nearly any mammalian gene cloning vector that allows a cloned gene to be expressed at high levels by transfected mammalian cells. By making use of the pre-pro-BDNF sequence that precedes the mature BDNF polypeptide, in place of the pre-pro-NGF sequence that precedes the mature NGF sequence, anterograde transport and release from the sensory neuron terminals within the BBB can be facilitated, in at least some types of neurons.

### Example 2

### Administration of Adenoviral NGF Vector to Olfactory Epithelium, to Deliver NGF to Cholinergic Neurons in the Basal Forebrain

Adenovirus-derived (or other virally-derived) vectors which carry a gene construct that encodes NGF (or some other CNS-active polypeptide) may be administered to the olfactory epithelium, of either a human patient (for medical purposes) or a test animal (for test or other research purposes), by using nasal instillation of an aqueous suspension of the vector, at a suitable titer concentration.

The aqueous carrier liquid should be compatible with adenovirus and olfactory epithelium vigor. Physiological saline (with buffering agents, if desired) can be used, and hypotonic or hypertonic solutions, or solutions containing any other component that may induce higher levels of viral transfection, can also be tested using routine experimentation. Articles such as Holtmaat et al 1996 provide information on dosage and administration techniques for efficient administration of adenoviral vectors via nasal instillation in mice; such procedures may be adapted for use in larger rodents such as rats or rabbits, or in other mammals, using methods known to those skilled in the art.

If desired, steps can be taken to increase (i) the extent and/or duration of contact between the fluid containing the gene vector and the olfactory neurons, and (ii) the receptivity of the neurons for taking in such genetic vectors. This can be done by, for example, administering a nasal decongestant to test animals (or human patients) a few hours prior to administration of the gene vectors, and by using an aqueous solution (and possibly a swabbing step, using a diluted solvent, such as isopropyl alcohol or acetone, that can help remove any mucous, oleaginous,, or other viscous coating) to rinse and/or clean the nasal sinuses immediately prior to vector administration. The receptivity of the olfactory neurons may also be increased by using mild mechanical abrasion, using a small wire loop, rounded spatula tip, or similar tool. In general, neuronal projections in a surface area which has become irritated to a point of mild inflammation tend to be more receptive to cellular uptake of foreign molecules than cells which can be regarded as being in a quiescent or resting state.

After allowing sufficient time for gene expression (usually in the range of about 24 to 72 hours), the effectiveness of gene vector delivery can be assessed by sacrificing some of the test animals, removing the olfactory epithelium, olfactory bulb, and basal forebrain, and processing each tissue type separately, to measure for the locations and concentrations of the vector-encoded polypeptide within that type of tissue.

The tasks of measuring and monitoring can be relatively simple where the vector-encoded polypeptide is distinguishably different from endogenous (presumably rodent) polypeptides. These types of analyses can use methods such as: (i) hybridization of cellular mRNA with DNA probes that are complementary to the vector mRNA sequences, but not to endogenous mRNA sequences, using procedures as described in articles such as Xian and Zhou 2000; (ii) techniques which use “polymerase chain reaction” (PCR) reagents and methods to detect DNA or mRNA sequences from the vector, as described in articles such as Chie et al 2000; and, (iii) immunostaining or similar methods which use monoclonal antibodies that selectively bind to the polypeptide or epitope tag encoded by the vector that was used, and do not bind to endogenous polypeptide in the test species (such antibodies are commercially available; they also can be prepared if desired, using methods disclosed in articles such as Conner 2000, Rush et al 2000, and Zhang et al 2000).

If desired, time-dependent levels of exogenous mRNA and/or polypeptide expression by transfected olfactory receptors can be measured by repeating one or more tests, over a range of times after administration of the genetic vector.

If desired, to provide control populations of cells and animals for purposes of data analysis, a genetic vector carrying a human (or epitope-tagged animal) polypeptide gene construct can be administered to the olfactory epithelium on one side of a test animal's nasal sinus, and a control vector which carries some other gene (such as a marker gene that encodes an easily-detected polypeptide) can be administered to the olfactory epithelium on the other side of the animal's nasal sinus. After the animal is sacrificed, histological and immunological examination of the left and right olfactory receptors, olfactory bulbs, and basal forebrain regions can be used to evaluate (i) polypeptide expression by the transfected olfactory receptors, and (ii) polypeptide transport and delivery by the BBB-straddling receptor cells to other classes of neurons located inside the BBB.

### Example 3

### Monitoring NGF Polypeptide Delivery to Cholinergic Neurons in the Basal Forebrain

The quantity of NGF polypeptide (or any other vector-encoded CNS-active polypeptide) which is delivered via transfected olfactory receptor neurons (or any other type of sensory neurons which straddle the BBB and which are transfected by a genetic vector as disclosed herein), into CNS regions that are entirely within the BBB, can be monitored in any specific class or cluster of neurons of interest, using histological and immunological analysis of cells and tissues from sacrificed test animals.

Well-established immunological procedures, such as immunohistochemistry and “enzyme-linked immunosorbent assay” (ELISA) tests, can be used to monitor and quantify the production, release, and distribution of human NGF (or any other CNS-active polypeptide encoded by a genetic vector that has been administered to an animal), at any location of interest within the CNS, following administration of a genetic vector to olfactory receptor neurons, or other type of sensory neuron. If human NGF is the vector-encoded polypeptide, monoclonal and polyclonal antibody preparations which recognize and bind to human NGF, but not to mouse, rat, or other rodent NGF, are commercially available, or may be generated using procedures that are well documented in the literature (e.g., Conner 2000; Rush and Zhou 2000). Methods for measuring NGF levels using immunohistochemistry are described in articles such as Conner et al 1992, and methods for measuring NGF levels using ELISA are described in articles such as Zhang et al 2000, and in manufacturer's instructions which are included in NGF-ELISA detection kits that can be purchased from commercial sources.

As briefly mentioned above, monitoring of a vector-encoded human polypeptide, in lab animals such as mice or rats, can be rendered simpler and more certain if monoclonal antibodies are used that recognize and bind to the vector-expressed (presumably human) polypeptide, but which do not bind to the mouse or rat version of the same polypeptide. Hybridoma cell lines which express monoclonal antibodies that selectively bind to human NGF but not mice or rat NGF have been created, and such monoclonal antibody preparations are available. Similar hybridoma cell lines which express monoclonal antibodies that selectively bind to human but not rodent forms of other CNS-active polypeptides have also been created, or can be developed using methods known to those skilled in the art.

Alternately or additionally, as noted above, it is possible to incorporate an epitopic tag (such as c-myc) in a neurotrophic gene construct sequence, to facilitate immunological detection of polypeptides expressed by that gene construct in transfected neurons, using methods described in articles such as Moller et al 1998. If such an approach is used, it is important to confirm that the polypeptide expressed by the vector construct is able to undergo all steps that are necessary for proper delivery to the neurons that are being targeted for treatment by that polypeptide.

For example, if the goal of a particular procedure is to deliver therapeutic polypeptides into the main cell bodies of basal forebrain cholinergic neurons, and an “epitopic tag” sequence is used to facilitate monitoring of the polypeptide, the targeted basal forebrain cholinergic neurons can be evaluated to ensure that they carried out uptake (such as receptor-mediated endocytosis) and retrograde transport of the vector-encoded, epitope-tagged polypeptides that were secreted by the transfected BBB-straddling neurons. These types of confirmatory tests can be carried out by using methods described in articles such as Altar & Bakhit 1991, Ferguson et al 1991, DiStephano et al 1992, and von Bartheld 2000, using reagents such as monoclonal antibodies that will bind specifically to the epitope tag sequence (and/or to a domain in the fusion polypeptide which contains part of the native amino acid sequence along with the tag sequence), to demonstrate that the vector-encoded polypeptides were taken up by the targeted basal forebrain cholinergic neurons.

If a vector-derived NGF carries an antigenic or epitopic tag, monoclonal antibodies which bind selectively to the tagged form may be creating using well-known methods, and can be used for any subsequent immunohistochemistry or ELISA measurements. If desired, confirmation that the delivery of NGF or some other CNS-active polypeptide (and its subsequent neurological and physiological effects on tissue inside the BBB) was indeed mediated by genetic vector administration to olfactory receptor neurons can be achieved by ablating these neurons in control animals, by treatment using a compound such as zinc sulfate solution, as described by Horowitz et al 1999.

Based on trans-synaptic tracer studies (e.g., Lafay et al 1991, Barnett et al 1993), it is generally anticipated that a vector-expressed polypeptide such as NGF will be detectable in the basal forebrain cholinergic neurons after allowing sufficient time for gene expression in the olfactory receptor neurons, which is likely to require a range of roughly 24 to 72 hours. This time period is anticipated to include anterograde transport of the polypeptide (after it has been expressed) to the receptor neuron's synaptic terminals in the olfactory glomeruli, secretion of the polypeptides within the glomeruli (roughly 8 to 24 hours), and transport of the polypeptides to and into basal forebrain cholinergic neurons (roughly 8 to 24 hours).

As discussed in Example 8, certain types of “trans-neuronal” vectors are also disclosed herein. That class of vectors may be able to be transmitted by sensory neurons to other types of neurons inside the BBB, in a manner which will transfect the recipient neuron with the foreign gene, causing one or more classes of “downstream” neurons lying wholly within the BBB to begin expressing the foreign therapeutic polypeptide.

However, unless specific steps are taken to provide “trans-neuronal” transport between neurons, it is assumed that: (i) transfection of initial or “primary” neurons (BBB-straddling neurons) by a genetic vector as disclosed herein will not lead to transmission of the vector to other non-primary neurons which are inside the BBB; and, (ii) transient gene expression will result, rather than permanent genetic transformation. If sensory neurons such as olfactory receptor neurons are used as the transfection targets, expression of the vector-borne gene(s) in such cells is expected to decrease substantially, and may stop entirely, during a period of several days to several weeks following the highest peak or plateau levels of polypeptide expression. If desired, reapplication of the same or a similar genetic vector to the olfactory receptor neurons can deliver an additional supply of NGF into BBB-protection CNS tissue.

### Example 4

### Monitoring Physiological Effects of NGF Delivery to Cholinergic Neurons in the Basal Forebrain

To enable and improve analysis of the actual physiological, behavioral, and other effects of CNS-active polypeptide delivery into BBB-protected CNS tissue using the genetic vector methods disclosed herein, experimental animals (or humans who have volunteered for clinical trials of this type of therapy) may be divided into two groups: (i) a test group which will receive a genetic vector which encodes NGF or some other selected neurotrophic or CNS-active polypeptide; and, (ii) a control group which receives a placebo treatment. The placebo treatment should use identical treatment of the instillation site (such as identical treatment with a decongestant, rinsing, swabbing, scraping, and/or other irritation of the nasal linings), followed by either: (a) nasal instillation of a plain saline solution with no genetic vector; or, (b) nasal instillation of a solution containing a genetic vector which might carry, for example, an innocuous and/or non-functional gene, a nonsense DNA sequence which does not encode any polypeptide, or a marker gene which encodes a polypeptide that can be easily detected if expressed in mammalian cells, but which has no significant physiological effect.

When animals are tested, introducing a CNS-active polypeptide (such as NGF or some other neurotrophic factor) through the BBB and into protected brain tissue may lead to either or both of at least two categories of observable differences: (i) effects on the neuroanatomy of the brain, which can be evaluated by histological, immunological, or other biochemical analysis of sections of brain tissue removed from animals that have been sacrificed; and, (ii) observable and measurable effects on the behavior of the animal.

A number of tests have been developed for assessing what appears to be happening inside a laboratory animal's CNS system, based on observable and measurable forms of behavior (such as the ability of treated mice or rats to remember what they encountered in prior challenges involving mazes, water mazes, etc.). In addition, other such tests are being developed, and any such test which is currently known or hereafter discovered can be used, provided that it is appropriate for assessing the physiological effects of the polypeptide on targeted neurons lying wholly within the BBB.

It should be noted that many of these tests involve a surgical or drug intervention which inflicts some type of damage on the animal's CNS, to model an injury, stroke, neurodegenerative disease, or other CNS disorder. Subsequent tests then seek to evaluate whether a certain treatment can help such animals recover from the inflicted injury or disorder.

One example of such a test is the “fimbria fornix lesion model”, a commonly used model in which the basal forebrain cholinergic neuron axons projecting to the hippocampus are “axotomized” (i.e., the main axon of a neuron is surgically severed; if untreated, this typically will cause the neuron to atrophy and die over a period of about two weeks or less, in most species). This induces atrophy and degeneration of the basal forebrain cholinergic neurons (e.g., Hefti 1994). However, that may not be a preferred injury model for evaluating the invention disclosed herein, because the basal forebrain cholinergic neurons which project through the fimbria fornix do not also project to the olfactory bulb.

Instead, preferred injury models for use herein should disrupt one of the neuronal pathways that is likely to be directly involved in, or affected by, the type of treatment disclosed herein. For example, surgically severing the cortical projections of neurons in the hindlimb of Broca (which project to both the olfactory bulb and the cortex) while leaving intact the olfactory bulb projection (so that retrograde axonal flow of NGF from the olfactory bulb is not blocked) would likely offer a better form of challenge to allow researchers to evaluation the effects of the treatments disclosed herein.

An alternative animal model approach to severing an axonal tract is to ablate a selected type of appropriate target tissue, in a manner which will deprive basal forebrain cholinergic neurons of endogenous NGF, thereby inducing atrophy and degeneration of basal forebrain cholinergic neurons. This approach can generally model the effect of excitotoxic injury associated with stroke. Published examples include Sofroniew et al 1993, which describes ablation of hippocampal tissue. This procedure can be modified to ablate entorhinal cortex tissue, which receives innervation from the hind limb of Broca (Wenk et al 1980), to induce atrophy of basal forebrain cholinergic neurons in the hindlimb of Broca.

The general methods described in Sofroniew et al 1993 may be used to monitor the effect of NGF delivery (using genetic vectors as disclosed herein, such as by transfection of olfactory receptor neurons) on the basal forebrain cholinergic neuron cell body size, and/or by evaluating levels of one or more enzymes or other polypeptides that will be directly affected by the presence and quantity of NGF, which can therefore be used as an indicator polypeptide (one example of a candidate enzyme that might be useful for such measurements is choline acetyl transferase, or ChAT; this enzyme is required for synthesizing acetylcholine). Another neuroanatomical effect that can be observed and measured when NGF is administered into the CNS (such as by ventricular injection or infusion) to test animals which have had selected regions of cortical CNS tissue ablated, axotomized, or otherwise challenged, is shown by the ability of the NGF administration to prevent or reduce injury-induced reductions in the density of ChAT-immunoreactive fibers in the surviving cortex material (e.g., Garofalo et al 1992). Accordingly, methods described in articles such as Garofalo et al 1992 can be used to monitor the effects of NGF delivery (arising from genetic vectors as disclosed herein), on the density of ChAT-immunoreactive fibers in the surviving cortex, after a surgical or other intervention which ablates or otherwise damages one or more selected regions of cortical material.

In addition to neuroanatomical changes, cortical ablation results in reduction in certain measurable behaviors that can distinguish between normal and impaired performance on various tests that require learning and memory in test animals. Examples include the “Morris water maze”, passive avoidance tests, responsive tests, and various other animal models, as described in articles such as Garofalo and Cuello 1994. It has been shown that administration of exogenous NGF into CNS tissue (such as by ventricular injection or infusion) in such test animals can significantly attenuate injury-induced deficits in behavioral performance by such animals. Accordingly, such behavioral, memory, and/or learning tests may be adapted and used to monitor the effects of NGF delivery into the forebrain region via genetic vector treatment of neurons which straddle the BBB.

Aging also results in a reduction in memory and learning performance, as measured by tests such as the Morris water maze, (Fischer et al 1987 and 1991), and age-related declines in memory and learning performance have been used in efforts to measure and quantify, for example, the loss of memory and learning that occurs in Alzheimer's disease (both in human patients, and in various animal models of Alzheimer's disease). It has been shown that administration of exogenous NGF into CNS tissue (such as by ventricular injection or infusion) can significantly attenuate at least some types of age-related declines in memory and learning (Fischer et al 1987 and 1991). Accordingly, the types of learning and memory tests used in such animal models can be used to monitor the effects of NGF delivery into the CNS by the methods and genetic vectors disclosed herein.

The monitoring and measuring methods mentioned in Examples 3 and 4 herein are not exhaustive or exclusive; they can be supplemented by other monitoring and measuring methods known to those skilled in the art, or hereafter discovered.

### Example 5

### Construction and use of Vectors Derived from Herpes Simplex Virus for Non-Invasive Delivery of NGF

The Examples above describe the use and evaluation of adenovirus-derived vectors, to genetically transfect BBB-straddling olfactory receptor neurons, as a route and method for non-invasive delivery of NGF into BBB-protected CNS tissue. However, it should be recognized that adenoviral vectors are not the only types of genetic vectors that can be used for non-invasive delivery of polypeptides into BBB-protected CNS tissue. Accordingly, Examples 5-8 describe the use of several other classes of genetic vectors.

To construct genetic vectors which are derived from herpes viruses, and which carry one or more “passenger” genes which encode CNS-active polypeptides as disclosed herein, methods described in articles such as Goins et al 1999 or Federoff et al 1992 can be used. HSV-derived vectors are capable of transfecting a wide variety of human cells, including olfactory receptor neurons, and they can induce transfected BBB-straddling neurons to express passenger genes, and to secrete significant levels of such polypeptides.

Accordingly, HSV-derived vectors may be used, if desired, in a manner directly comparable to the use of adenovirus-derived vectors as described in Examples 1-4. The methods described in Examples 2-4 (supplemented when appropriate by other or additional methods known to those skilled in the art) may be used for nasal instillation of HSV-derived vectors, and for post-transfection monitoring and evaluation.

### Example 6

### Construction and use of Lipid-Based Vectors for Non-Invasive Delivery of NGF

Methods for preparing DNA plasmid-lipid complexes designed for transfecting mammalian cells are described in numerous publications, such as the chapter by Nabel (pp. 127-133) in Methods in Molecular Medicine: Gene Therapy Protocols (P. Robbins, ed., 1997). A range of liposome preparation kits designed for use in gene transfer are available commercially, or can be designed and created by any skilled technician using published methods. The preferred choice of lipid formulation and preparation parameters (including lipid and DNA concentrations and ratios in the preparation mixture, temperature, etc.) for use with a particular size, type, or concentration of plasmid DNA or other DNA preparation can be determined by routine testing of various preparative mixtures, using parameter ranges and confirmatory tests that are discussed in numerous articles and in the instructions that accompany most commercially available kits.

Administration of lipid-based genetic vectors to olfactory epithelium, via nasal instillation of a lipid-gene complex suspended in an aqueous saline solution or other carrier liquid, can use the same general procedures described in Example 2, adapted for such use by means known to those skilled in the art. The methods described in Examples 3 and 4 (supplemented when appropriate by other or additional methods known to those skilled in the art) may be used for post-transfection monitoring and evaluation.

### Example 7

### Construction and use of DNA Vectors that Target Endocytotic Receptors; Cyclic Ligand Selection Process

The general principals and procedures that can be used to prepare “receptor-targeting gene vectors” are described in various publications, such as the chapter by Findeis (pp. 135-152) in Methods in Molecular Medicine: Gene Therapy Protocols (P. Robbins, ed., 1997). As described previously, these vectors contain ligands that will bind to certain types of receptors that are exposed on the surfaces of neurons.

As mentioned in the Background section, a number of known types of neuronal surface receptors undergo a process called “endocytosis”, after a ligand molecule becomes bound to the receptor. As suggested by the name, an endocytotic receptor will be taken inside the cell, after its ligand molecule becomes bound to it. This type of activity can be shown by tests using radiolabelled ligands.

One example of an endocytotic receptor is the “p75” receptor, which is accessible on the surfaces of various types of sensory neurons (including olfactory receptor neurons). It has been shown to exist in both humans and rats, making it highly useful for various types of research. Also, known as the p75NGFR receptor (where NGFR indicates “nerve growth factor receptor”), it is of special interest for use as described herein, because it has been shown to be “up-regulated” (i.e., the expression of mRNA encoding the receptor is increased, and the number of receptors that appear on the surfaces of the neurons is increased) in various types of neurons that are subjected to crisis or stress conditions. As examples, p75 expression increased in the motor neurons of rats following a peripheral nerve injury (Yan et al 1988), and p75 expression also increased in the motor neurons of human patients suffering amyotrophic lateral sclerosis (Seeburger et al 1993).

Selection and/or creation of an appropriate ligand that will bind to an endocytotic receptor on the type of neuron being targeted (ideally, in the species being tested or treated) is key to construction of an effective receptor-targeting gene vector of this type. A variety of such ligands are already known, and others can be created, using methods such as briefly summarized above.

Since endocytotic receptors are proteins, it is usually possible to create a complementary polypeptide that will bind to any such receptor, by using a polypeptide sequence from the receptor as an antigen, during the creation of monoclonal antibodies. Using well-known techniques, the antigenic sequence derived from the receptor is injected into animals such as mice, rats, or rabbits; the resulting antibody-producing cells are then fused with an immortalized cell line; and, the resulting “hybridoma” cells are screened, to identify and isolate a clonal cell line which secretes monoclonal antibodies that will bind with high affinity to the receptor of interest. Once the desired monoclonal antibody line has been created and identified, a smaller domain or fragment usually can be isolated from the variable binding portion of that monoclonal antibody (often referred to by acronyms such as the “scFv” fragment, where “sc” refers to “single chain” and “Fv” refers to the variable fragment, which comprises the binding domain). A gene sequence which encodes that binding fragment can then be incorporated into a plasmid or other vector, to allow unlimited quantities of the receptor ligand fragment to be synthesized by fermentation of host cells.

Using that general type of approach, monoclonal antibodies can be created which will bind to essentially any type of known endocytotic receptor, on any type of sensory neuron having one or more peripheral projections.

To continue the example of the p75 receptor, a monoclonal antibody known as 192-IgG (described in Chandler et al 1984), has been shown to bind with high affinity to p75 receptors in rats. The 192-IgG monoclonal antibody (as well as various fragments derived from it) has been shown to undergo endocytosis, and retrograde transport, in peripherally-projecting neurons which express the p75 receptor (Yan et al 1988).

Once a polypeptide sequence that can serve as a receptor-binding ligand has been identified, various reagents can be used to temporarily couple a DNA segment to the ligand polypeptide, thereby creating a polypeptide-DNA complex. In one preferred method, a polypeptide can be created having “polylysine” domain, with a series of lysine residues (or other amino acid residues having a positive charge at physiological pH) coupled to the ligand polypeptide. This can be done by any of several methods, such as adding a string of lysine codons to the polypeptide-encoding gene which is used to express the polypeptide in a host cell, or chemically conjugated polylysine to a polypeptide by using bifunctional crosslinking reagents. Because of chemical attraction (a polylysine strand has a positive charge, and a DNA strand has a negative charge), DNA segments will adhere in a non-covalent manner to polypeptides that have polylysine “tails”. When the ligand portion of the polypeptide binds to an endocytotic receptor, it commences a process that will draw the entire polypeptide-polylysine-DNA complex (plus the receptor protein as well) into the cell. After that complex has been taken inside the cell, the DNA molecule will be released, and can be retrogradely transported inside the cell.

Alternately, a normal ligand for a neurotrophin receptor (such as NGF, which binds to NGF receptors) may be used. Radiolabelled NGF has been shown to undergo receptor-mediated internalization and retrograde transport, in sensory neurons which express a receptor called “trkA” (e.g., Barbacid 1995); accordingly, the NGF polypeptide itself can be used as a ligand, for cells having that receptor. It should be noted that the NGF polypeptide itself (and, it is believed, various other neuronal receptor ligands) has a net positive charge, similar to the positive charge found on histones, a class of DNA-binding proteins that are associated with chromosomes; accordingly, the need to first attach a polylysine or other similar positively-charged domain or conjugate to an NGF or other positively-charged polypeptide might be avoided entirely, or minimized, and the polypeptide itself may be able to function as both a receptor targeting ligand and a DNA carrier.

Various other known compounds also provide good candidates which can be evaluated for potential use as ligands which can bind to endocytotic receptors on one or more types of BBB-straddling neurons, and/or as peptide sequences which can promote retrograde transport or some other function after a genetic vector or some portion thereof has been taken inside a neuron. As just one example, Wiley and Lappi 1993 describes a conjugate formed by coupling (i) monoclonal antibody 192-IgG, which binds to the p75 neuronal receptor, to (ii) a plant-derived toxin called saporin, which is believed to promote intracellular transport to a neuronal cell body, where it inactivates ribosomes. This conjugate was reported by Wiley and Lappi to be useful for causing selective and targeted neuronal lesions, for research purposes; accordingly, it can be regarded herein as a “probe” compound. It may be possible, using methods known to those skilled in the art, to modify and adapt that or similar types of probe compounds, to render them nonlethal to neurons, in a manner that will allow a segment of DNA to be (i) non-covalently coupled to the “probe” compound; (ii) transported to the neuronal cell body, with the aid of the probe compound, and then (iii) released from the probe compound, into the cell body, after the internalization and transport steps have been completed.

In addition, numerous types of neurotoxins have been derived from the venoms of spiders, wasps, snakes, marine snails, and other venomous organisms. One of the more common features of neurotoxins is that they bind (often with extremely high levels of binding affinity) to one or more types of receptors and/or ion channels on the surfaces of neuronal projections; this is one of the principle mechanisms that organisms in the wild use to paralyze or otherwise incapacitate their prey, and to defend against attackers. A substantial number of such neurotoxins have been modified by research biochemists, to form analogs, conjugates, and other variants and derivatives which have lower, non-toxic levels of binding affinity for neuronal receptors or ion channels, in the search for therapeutic agents that can be medically useful without inflicting pain at the levels normally caused by a bite or sting from a highly venomous animal.

In addition, various types of toxins from pathogenic microbes (including tetanus toxin, cholera toxin, etc.), and various compounds-derived from plants (including compounds that fall within a category known as lectins or agglutinins) offer a number of candidates for use as described herein, which can be evaluated to determine whether that can provide selective ligands that will help a genetic vector bind to and transfect one or more types of BBB-straddling neurons.

Accordingly, any such known or hereafter-discovered neurotoxin, microbial toxin, plant lectin, or other functionally similar compound which can bind selectively to one or more receptors, ion channels, proteins, glycoconjugates, or other molecules that are found on the surfaces of BBB-straddling neurons, and any analog, conjugate, or other variant or derivative of any such neuron-binding compound, can be evaluated for potential use as disclosed herein, as a ligand that may help genetic vectors selectively bind to and transfect one or more types of BBB-straddling neurons.

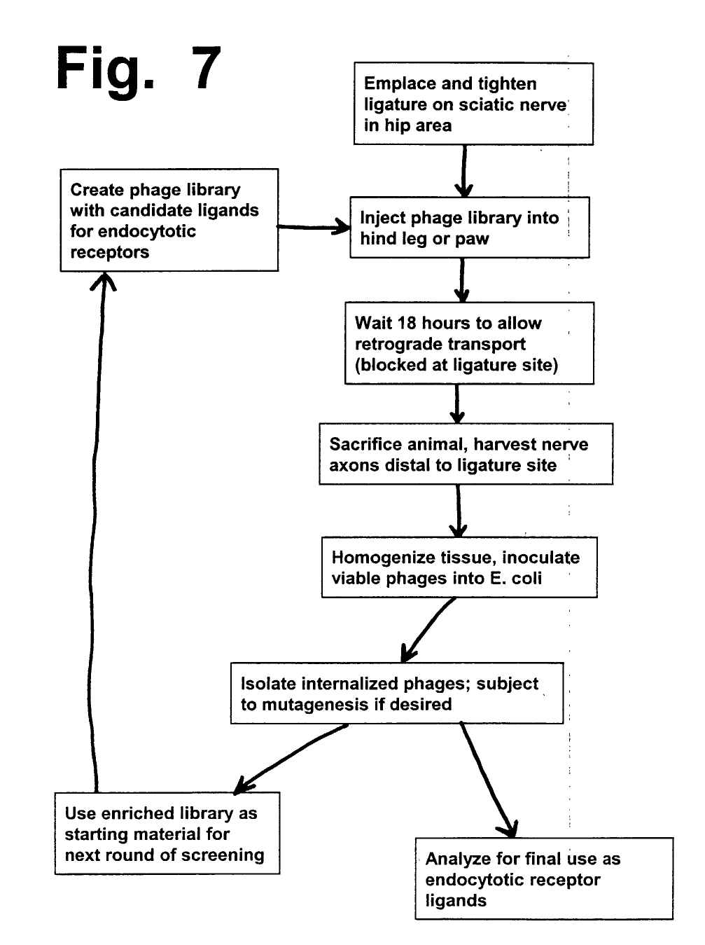

As a third alternative approach, a “combinatorial chemistry” approach which uses phage display libraries can be used, as described in the Preferred Embodiments section, and as illustrated in FIG. 7. Briefly, this approach uses phage display libraries to generate an array of potential ligand polypeptides, wherein each phage expresses a single polypeptide fragment. The entire array of phages is screened, using a process of receptor-mediated endocytosis carried out by neurons in vivo, such as in rats that have had ligature-type constrictions placed on nerve bundles such as a sciatic nerve. Intact phage particles (which can infect host bacteria) are subsequently harvested from a segment of extracted nerve cells, immediately adjacent and “distal” to the point where the ligature was placed. The harvested phage particles are then inoculated into E. coli cells, which are used to grow a subsequent generation of phages. This new generation of phages will contain those particular phages which were present inside the nerve cells, at the location adjacent to the ligature. As described in more detail in the Background section, because of how these phages were inoculated into and subsequently harvested from a treated lab animal, the particular phages which are selected by this screening process presumably must express ligand polypeptide sequences which caused the selected phages to be: (i) taken inside the peripheral projections of neurons, by a process of endocytosis; and, (ii) retrogradely transported, inside the neuronal axon, to the site adjacent to the ligature.

By repeating that type of screening and selection process several times (and by subjecting phage lines which performed well to random or targeted mutagenesis, if desired), clonal phage lines can be identified which will have incorporated genes that encode polypeptide sequences which are highly effective as ligands that can bind to endocytotic receptors and trigger endocytosis. Those phages can be analyzed, and the gene and amino acid sequences of those ligands can be determined, for subsequent use in creating highly effective receptor-targeting transfection vectors.

If desired, this type of cyclic screening process can be further enhanced by the use of various types of assays, such as “fluorescent activated cell sorting” (FACS), which can be carried out using known types of machines that are usually called “flow cytometers”. As an example of how this process can be used, plasmid, phage, or “phagemid” DNA can be fluorescently labelled, using a compound such as rhodamine. Cells grown in tissue culture, which are known to have p75 receptors on their surfaces, are incubated with various polypeptide-DNA complexes, and the cells are then passed through a cell sorting machine equipped with a fluorescent detector. If the fluorescent light emitted by a particular cell is relatively strong, that will indicate that a certain cell took in large quantities of labelled polypeptide-DNA complexes, and a synchronized very brief jet of gas or fluid can cause the fluid stream carrying that particular cell to be diverted into a separate collection device. In this manner, cells which contain large quantities of “uptaken” DNA can be isolated rapidly and easily, using high-speed automated equipment, and can subsequently be reproduced, analyzed. or otherwise processed to evaluate or replicate the types of polypeptides which caused high levels of polypeptide-DNA uptake into the cells. This is just one example of how automated analysis and manipulation can be used to simplify and enhance this invention, and other methods will also become apparent to those skilled in the art.

As yet another alternative, DNA-protein complexes may be prepared by coupling a DNA segment to a known type of capsid protein derived from adenovirus (or from various other viruses which act in a similar manner). This adenoviral capsid protein is known to help promote efficient release of the vector-carried DNA from the endocytotic vesicle, after the complex has entered the target cell. This approach is described in the chapter by Curiel (pp. 25-40) in Methods in Molecular Medicine: Gene Therapy Protocols (P. Robbins, ed., 1997). If desired, the adenoviral capsid protein sequence and the receptor-binding ligand sequence may be combined, in a fusion protein.

Nasal instillation of these types of receptor-targeting transfection vectors can use the same general procedures described in Examples 2 and 6; those general procedures can be adapted specifically for use with receptor-targeting ligand vectors, by means known to those skilled in the art. The methods described in Examples 3 and 4, and other methods known to those skilled in the art, can be used for post-transfection monitoring and evaluation.

### Example 8

### Construction and use of Trans-Neuronal Vectors to Deliver NGF Gene to Second-Order Neurons in the Brain

This example describes a method of developing “transneuronal” gene vectors, which may be able to transport foreign genes (rather than just polypeptides expressed by foreign genes) into certain classes of neurons that are located entirely within the BBB. In other words, the goal of such transneuronal gene vectors is to transfect, and genetically transform, not just neurons which straddle the BBB, but also other “secondary” or “second order” neurons which share synaptic junctions with transfected BBB-straddling “primary” neurons (and, potentially, tertiary neurons as well, which share synaptic junctions with secondary neurons). Transneuronal vectors may be able to greatly increase both the quantity and the distribution of new and/or supplementary polypeptides, which can be secreted by neurons that reside wholly within the BBB. Such treatment may become an effective way to treat certain neurodegenerative diseases characterized by widespread and disseminated brain damage, such as Parkinson's disease and Alzheimer's disease.

It should be recognized that these types of transneuronal vectors are not expected to replicate and produce multiple copies of themselves, after they enter secondary (or tertiary, or subsequent) neurons. Instead, the goal of such transneuronal vectors is simply to place these vectors in “downstream” neurons which are fully within the BBB, rather than limiting their placement to “primary” neurons which straddle the BBB. However, it is recognized that in some cases (which will become more probable and more frequent if additional steps are taken to promote integration of the gene sequences into chromosomes within the downstream neurons, by steps such as making use of terminal repeats derived from adeno-associated virus to bracket the transcription unit of the plasmid), this approach may lead to non-invasive methods of genetic therapy on CNS neurons that are entirely within, and protected by, the blood-brain barrier.

The currently anticipated approach to constructing transneuronal vectors arises out of various facts known about transneuronal transport of certain pathogens and polypeptides, including: (i) the “nontoxic fragment C” of tetanus toxin (e.g., Knight et al 1999); (ii) barley lectin (Horowitz et al 1999); and, (iii) wheat germ agglutinin (Yoshihara et al 1999). Barley lectin and wheat germ agglutinin have been shown to undergo active transneuronal transport from olfactory receptor neurons, into basal forebrain cholinergic neurons, and into other classes of neurons as well, including neurons in the locus ceroleus and raphe nuclei. Other polypeptide sequences with transneuronal transport capability can also be discovered and created, using the same type of combinatorial selection strategy described in Example 7, above; this can be done by repeated cyclic testing of phage display libraries, to identify phages which have gene sequences that encode polypeptides which drive transneuronal transport of phage into secondary (or tertiary, or subsequent) neurons.

Known methods can be used to construct a gene vector using a polypeptide which is known to have transneuronal transport capability. For example, polylysine can be covalently bonded to a transport polypeptide, using a chemical reaction, or using a genetically modified gene which encodes for numerous lysine (or other positively charged) residues at one end of the polypeptide. Since polylysine is positively charged, it will attract DNA segments (which are negatively charged) when the polypeptide is mixed with DNA segments in solution. This leads to moderately strong but noncovalent binding of the DNA to the polypeptide. This type of preparation is described in Knight et al 1999.

Administration of these genetic vectors to olfactory epithelium, via nasal instillation of vectors suspended in an aqueous saline solution or other carrier liquid, can use the same general procedures described in Example 2, adapted for such use by means known to those skilled in the art.

The methods described in Examples 3 and 4 (supplemented when appropriate by other or additional methods known to those skilled in the art) may be used for post-transfection monitoring and evaluation. Demonstration of expression of transneuronally transported NGF gene constructs within neurons inside the BBB (such as basal forebrain cholinergic neurons) can be done with sensitive procedures such as in situ hybridization and/or polymerase chain reaction (PCR). As described in Example 3, detection of expression will be facilitated by making use of NGF gene sequences which express polypeptides that are distinguishably different from the corresponding polypeptides in the host species.

Additional monitoring of the physiological and behavioral effects caused by such transneuronal vectors can be done by procedures such as described in Example 4.

### Example 9

### Construction and use of Viral Vectors to Deliver Anti-NGF Polypeptides into Dorsal Horn Regions in the Spinal Cord

While Examples 1-8, above, describe delivery of neurotrophic polypeptides such as nerve growth factor (NGF) to help stimulate neuronal growth or repair, this Example 9 and several following examples describe a completely different type of polypeptide, referred to herein as an “anti-NGF” polypeptide. Such polypeptides, if they are transported through the BBB and delivered into properly targeted locations, can help treat and reduce various conditions, such as neuropathic pain or autonomic dysreflexia. Such polypeptides can work by at least two known mechanisms: (i) by binding to NGF molecules in cerebrospinal fluid (CSF) or synaptic fluid, thereby inactivating the NGF molecules by rendering them unable to bind to NGF receptors; and/or, (ii) by binding to NGF receptor proteins on neurons, thereby occupying those receptors and rendering them inaccessible to NGF molecules, in a manner which does not trigger the cellular reactions that occur when free NGF molecules bind to the receptors.

Anti-NGF polypeptides which bind to free NGF molecules in CSF or synaptic fluid are known, and are described in articles such as Ruberti et al 1993 and 1994. Gene sequences which encode these anti-NGF polypeptides are also known, and can be incorporated into complete gene constructs having suitable gene promoters which will drive gene expression in mammalian cells, as disclosed in Example 1 above.

If such a gene construct is intended for expression in a nocioceptive neuron, rather than an olfactory receptor or other type of sensory neuron, attention must be given to the gene promoter that will be used to drive expression of the gene. A range of potential gene promoters that function in nocioceptive neurons are known, and suitable promoters which can drive desired levels of gene expression in nocioceptive neurons can be selected from those candidate promoters by routine experimentation. Preferred promoter selection also can be influenced by expression levels that occur naturally in different types of neurons.

For example, by using a gene promoter which drives expression of the CGRP gene (described in Watson et al 1995), expression of a vector-carried anti-NGF gene can be restricted to transfected nocioceptive neurons which express the neuropeptide CGRP. The CGRP promoter may be especially useful if one objective of a treatment is to reduce the level of NGF in the spinal cord of a patient suffering from neuropathic or other chronic pain, because the activity of this promoter is enhanced in the presence of NGF. Thus, when NGF levels in the spinal cord increase, this promoter may be able to increase synthesis and secretion of anti-NGF in response, thereby creating a self-limiting, self-regulating gene expression system.

A gene construct having an anti-NGF coding sequence and a selected promoter can be placed in a gene vector derived from replication-deficient adenoviruses, using methods such as described and cited in Example 1 and Ruberti et al 1993 and 1994, or in a gene vector derived from replication-deficient herpes viruses, using methods such as described in Example 5 and the articles cited therein.

Since this example relates to methods of reducing and controlling NGF-aggravated neuropathic pain, the anti-NGF vectors disclosed herein will not be inserted into sensory receptor neurons such as olfactory neurons. Instead, these vectors will be injected into regions that contain high concentrations of nocioceptive nerves, including subcutaneous tissues or muscle tissue in regions that are (i) plagued by neuropathic pain, such as allodynia, and/or (ii) innervated by BBB-straddling nocioceptive neurons, using methods such as described by Sahenk et al 1993, or as determined to be optimally effective during clinical trials or during a treatment procedure involving a specific patient.

After injection into regions with nocioceptive nerve projections, at least some of the anti-NGF gene vectors will contact nocioceptive neuron projections outside the BBB. Using the same mechanisms that viruses normally use to inject DNA into cells during infection, the viral vectors (or analogous mechanisms used by non-viral vectors, as described in Examples 6-8) will inject the genetically-engineered vector DNA into the outer projections of the BBB-straddling nocioceptive neurons that innervate those tissues. Using retrograde transport, the vector DNA will be carried through the cell cytoplasm into neuronal regions called dorsal root ganglions, which flank the spinal cord. The gene which encodes the anti-NGF polypeptide will be expressed in the nocioceptive neurons residing within the ganglions (the plural form is also spelled “ganglia”), thereby creating anti-NGF polypeptide molecules within BBB-straddling neurons.

At least some of these anti-NGF polypeptides will be transported, using normal anterograde transport, through the axons of BBB-straddling neurons, until they reach synaptic terminals or similar exit points which are located in “dorsal horn” regions (and possibly other regions) of the spinal cord, in tissue that is enclosed within and protected by the BBB. The efficiency of this anterograde transport step may be enhanced by including a leader or signal peptide, at the start of the secreted or mature polypeptide, that will instruct the transfected cell to package the polypeptide in vesicles containing neurotransmitter peptides destined for anterograde transport and release at synapses lying within the BBB. An example of such a leader sequence suitable for use in nocioceptive neurons is the pre-pro-BDNF sequence, which directs nocioceptive neurons to anterogradely transport mature BDNF (which these neurons normally synthesize) into dorsal horn regions within the spinal cord.

When released by nocioceptive neuron terminals at these locations, the anti-NGF molecules will bind to NGF molecules, thereby reducing the amount of free NGF in the spinal tissue at that location. Reduced access to NGF does not kill mature nocioceptive neurons; however, it does lead to a reversible form of suppression (often called “down regulation”) of nocioceptive functions and activity, as demonstrated by direct administration of anti-NGF into laboratory animals, as described in articles such as Christensen et al 1996.

Anti-NGF may be detected in the spinal cord after allowing sufficient time for gene expression in the dorsal root ganglion (anticipated to be about 24 to 72 hours) and anterograde transport and release of the anti-NGF into the dorsal horn regions of the spinal cord (anticipated to be another 8 to 24 hours).

In laboratory animals that have been sacrificed, the release of anti-NGF polypeptides in the spinal cord can be monitored directly with appropriate methods, such as electron microscopy, immunological staining, and various methods of labelling the vector-derived anti-NGF, such as inclusion of an appropriate antigenic tag in the amino acid sequence of the polypeptides encoded by the vector gene. It also may be possible to monitor the concentration and/or effects of anti-NGF by measuring the concentration of free NGF. When anti-NGF is released, it will bind to and neutralize endogenous NGF in the spinal cord. Accordingly, changes in the level of NGF in the spinal cord can be monitored by making use of various immunological procedures, such as those outlined in Example 3, and applying these procedures to examination of the spinal cord tissue receiving innervation from the transfected sensory neurons. Changes in the level of NGF in the spinal cord can also be inferred from characteristic neuroanatomical and behavioral changes, as outlined in more detail in Example 10.

### Example 10

### Monitoring Physiological and Behavioral Effects of Anti-NGF Delivery into Dorsal Horn Regions in the Spinal Cord

To allow for statistical comparison of test results, experimental animals may be divided into two groups. One group (the test animals) will receive the anti-NGF gene vector, as described above, while the other group (the control group) will receive a vector containing noncoding DNA, an innocuous gene, or a marker gene that has no known neuroactive effect on CNS tissue. The physiological effects of administering anti-NGF into the spinal cord include effects on the neuroanatomy of the brain and effects on the behavior of the animal, as discussed in articles such as Christensen et al 1996 and Christensen et al 1997.

Appropriate choice of animal model(s) is important in evaluating the physiological effects of anti-NGF delivery into the spinal cord. In addition to animal models of inflammatory pain, various models of hyperalgesia and “allodynia” (chronic and/or neuropathic pain, in which moderate stimuli are interpreted by the CNS as severe pain) can be generated by injuring or challenging a peripheral nerve.

As just one example of a common method for modelling neuropathic pain, and for testing potential treatments for neuropathic pain, a loop (“ligature”) of suture thread can be surgically placed and then tightened around half to two-thirds of the sciatic nerve, in one of the hind legs of an animal such as a rat. Within several days to a week, the leg will become hyper-sensitized and susceptible to serious pain in response to mild stimulus. Rats that have been treated in that manner can then be placed in a special box with automated sensors, which can measure and record how many seconds pass after the floor plate begins to warm up, until the rat lifts the hyper-sensitive paw to get it off the warmed surface. If a candidate treatment can extend the average number of seconds that pass before nerve-ligated paws are raised, until those values approach the comparable times for unoperated control animals, that indicates that the treatment may be effective in reducing neuropathic pain, and may deserve more elaborate testing on larger animals, and/or clinical trials on human patients.

It should be recognized, however, that peripheral nerve ligation or other challenges or injuries may interfere with retrograde transport of vector DNA from an injection site to the dorsal root ganglion, and may complicate the design and interpretation of such tests for use to evaluate the therapies disclosed herein. Accordingly, challenges which directly affect and involve the spinal cord may be preferred in at least some situations. Chronic or neuropathic pain due to spinal injury is frequently observed in clinics, and over-production of NGF within the spinal cord has been implicated in a condition known as “primary afferent sprouting”, which often follows spinal cord injury (e.g., Krenz et al 2000). Accordingly, spinal cord injury models which reproducibly causes hyperalgesia or chronic pain can be used to monitor the effects of anti-NGF delivery into the spinal cord on hyperalgesia or chronic pain. Examples include the spinal cord injury model described in Krenz et al 1999, which is believed to be suitable for evaluating the physiological effects of anti-NGF delivery into the spinal cord using the methods disclosed herein.

Neuroanatomical changes in the pattern of sensory innervation of the dorsal horn associated with hyperalgesia or chronic pain includes abnormal sprouting of “A” fibers and nocioceptive CGRP-containing fibers into lamina II of the dorsal horn (e.g., Bennet et al 1996). The effect of administration of an anti-NGF gene vector on such neuroanatomical changes can be monitored by using the methods described by Bennet et al 1996 or Christensen et al 1997, while using an agent such as cholera toxin B subunit to transganglionically label the A fibers, and by using CGRP immunohistochemistry to visualize the nocioceptive (NGF-responsive) fibers in the dorsal horn.

Alternately, the effect of delivery of anti-NGF on the number and extent of synaptic connections between peripherally-projecting sensory neurons and second-order neurons in the spinal cord may be evaluated by transneuronal tracing methods, using procedures such as described in Blessing et al 1994.

Established methods for functional monitoring of pain responses in animal models include: (i) measuring foot withdrawal latency in response to thermal stimulus, as briefly summarized above and described in more detail in articles such as Romero et al 2000; (i) measuring responses to application of von Frey hair stimulus, as described in articles such as Deng et al 2000.

Another condition that may be susceptible to treatment by anti-NGF or comparable polypeptides is often referred to as “autonomic dysreflexia”. This condition occurs most commonly in patients with spinal cord injuries in the upper thoracic or cervical region, which disrupt the normal set of connections in the preganglionic sympathetic neurons in the upper spinal cord. As a result, the normal feedback control mechanisms in the autonomic nervous system can be disrupted, in ways that can cause a normal stimulus (such as colon distension, indicating full bowels) to trigger other reflex results, some of which can be potentially life-threatening (such as increases in heart rate and blood pressure, to levels which can pose major risks of severe cardiovascular consequences, such as a heart attack or stroke).

As shown in animal model studies (e.g., Krenz et al 1999), direct infusion of anti-NGF into the spinal cord in animal models of this condition can prevent or help control this type of unwanted reflex response, by helping suppress abnormal and unwanted innervation of the dorsal horn by nocioceptive neurons, as can occur after that type of spinal cord injury.

Therefore, the use of this invention to introduce anti-NGF polypeptides into spinal regions that are protected by the BBB, by using genetic vectors to transfect exposed and accessible projections of nocioceptive neurons (and possibly other spinal neurons, including motor neurons), holds substantial promise in treating autonomic dysreflexia.

### Example 11

### Construction and use of Lipid-Based Gene Vectors to Deliver Anti-NGF Polypeptides into Dorsal Horn Regions in the Spinal Cord

The procedures described in Example 9 and articles cited therein may be used to create an anti-NGF gene construct that will be expressed in one or more targeted classes of nocioceptive neurons. The resulting gene construct copies can be placed inside a DNA vector such as a plasmid or other stable form, using known methods. The DNA vectors can then be placed inside lipid vesicles (liposomes), using methods such as described in Example 6 and in the chapter by Nabel (pp. 127-133) in Methods in Molecular Medicine: Gene Therapy Protocols (P. Robbins ed., 1997).

The resulting liposome vectors may be administered to peripheral projections of nocioceptive neurons by means such as injecting an aqueous suspension of liposomes in saline solution into regions that contain high concentrations of nocioceptive nerves, including subcutaneous tissues or muscle tissue in regions suffering from neuropathic or other chronic pain, using methods such as described by Sahenk et al 1993, or as determined to be optimally effective during clinical trials or during a treatment procedure involving a specific patient. At least some of the liposome vectors will adhere to and merge with nocioceptive neuron projections which are located outside the BBB, in the muscle tissue, and that reaction will deliver the anti-NGF encoding DNA which was carried by the liposomes into the neuronal projections. Retrograde transport of the DNA segments will carry at least some of the anti-NGF DNA to the neuronal cell body, where the anti-NGF genes will be expressed into anti-NGF polypeptides.

Subsequent delivery of the anti-NGF polypeptides into the spinal cord, and the physiological and behavioral effects of the anti-NGF polypeptides in spinal tissue, can be measured and monitored using procedures such as described in Examples 9 and 10, above.

### Example 12

### Construction and use of DNA Vectors that Target Endocytotic Receptors on Nocioceptive Neurons, to Deliver Anti-NGF Polypeptides into Dorsal Horn Regions in the Spinal Cord

Various known methods, such as those described in Example 9, can be used to create anti-NGF gene constructs that will be expressed in nocioceptive neurons. Other known methods, such as those described in Example 11, can be used to place the anti-NGF gene constructs into plasmid form or other stable forms that can be transported into nocioceptive neurons by non-viral vectors such as liposomes.

These stable DNA forms can then be-used to form protein-DNA complexes, which incorporate polypeptide segments that will bind in a specific manner, as ligands, to nocioceptive neuron receptors which undergo endocytosis, using procedures such as described in Example 7. Various such receptor-specific polypeptide segments are already known, and others can be identified and developed using the phage library approach described in Example 7.

Accordingly, these steps, when compiled together in proper sequence, will create genetic vectors that can specifically target endocytotic receptors on peripheral projections of nocioceptive neurons, in regions that are not enclosed within the BBB and which therefore provide relatively simple access to the peripheral projections. Such receptor-specific endocytotic gene vectors can be used to transfect such neurons with anti-NGF genes that will be expressed in those neurons, and the neurons themselves will then deliver and secrete the anti-NGF polypeptides in dorsal horn regions of the spinal cord, in a manner which can help control and reduce neuropathic pain and possibly other pain disorders.

Delivery of such anti-NGF polypeptides into spinal cord tissue can be monitored using procedures such as described in Example 9, and the physiological and behavioral effects of anti-NGF polypeptides in such spinal cord tissue can be evaluated using procedures such as described in Example 10.

### Example 13

### Trans-Neuronal Anti-NGF Vectors that will Transport Anti-NGF Gene to Second-Order Neurons in the Spinal Cord

Gene constructs (including suitable gene promoters) that can express anti-NGF polypeptides in nocioceptive neurons can be created as described in Example 9. These gene constructs can be placed in plasmids or other stable forms, using known methods.

Such anti-NGF plasmids or other DNA vectors can then be coupled to “transneuronal polypeptides” that can help enable and promote the transfer of a protein-DNA complex from one neuron, to another. Various such “transneuronal polypeptides” are known, and include, for example, the “nontoxic fragment C” of tetanus toxin (e.g., Knight et al 1999), barley lectin (Horowitz et al 1999), wheat germ agglutinin (Yoshihara et al 1999) (also listed in Example 8, above).

By binding reversibly to proteins that are (i) exposed at synaptic terminals in the periphery, and (ii) internally transported by the BBB-straddling neuron to synapses within the BBB, such “transneuronal polypeptides” can enable the transport of protein-DNA complexes through the cytoplasm of transfected “primary” BBB-straddling nocioceptive neurons, through the BBB, and into neuronal terminals located in the dorsal horn, and possibly elsewhere in spinal tissue. The transneuronal proteins will then help the protein-DNA complexes exit the synaptic terminals of the BBB-straddling nocioceptive neurons, and enter into adjacent spinal cord neurons that are located entirely within the BBB, thereby transfecting “secondary” spinal cord neurons protected by the BBB.

Delivery of such anti-NGF gene vectors and encoding sequences into BBB-protected “secondary” spinal neurons can be monitored by methods such as in situ DNA probe hybridization and PCR analysis, using spinal cord cells and tissue from sacrificed lab animals. Expression of anti-NGF polypeptides by transfected “secondary” spinal neurons can be monitored using procedures such as described in Example 9, and the physiological and behavioral effects of anti-NGF genes and polypeptides in such spinal cord cells can be evaluated using procedures such as described in Example 10.

### Example 14

### Use of Adenoviral Vectors for Transfecting Spinal Motor Neurons that will Transport Neurotrophic Polypeptides to Upper Motor Neurons

Procedures for preparing non-pathogenic adenoviral vectors that cannot replicate in normal cells have been published in articles such as Graham and Prevec 1995. Articles that describe examples of such vectors which contain genes that encode various neurotrophic factor include Dijkhuizen et al 1997, Gravel et al 1997, Baumgartner et al 1998, and Romero et al 2000.

Gene sequences which encode “glial cell line derived neurotrophic factor” (GDNF), and which have been isolated and put into conveniently-handled forms such as plasmids or adenoviral vectors, are described in articles such as Lindsay et al 1996, Choi-Lundberg et al 1997, and Baumgartner et al 1997, and in various other articles cited therein.

Gene sequences which encode “neurotrophin-3” (NT-3) and which have been isolated and put into conveniently-handled forms such as plasmids or adenoviral vectors, are described in articles such as Snider 1994 and Bothwell 1995, and in various other articles cited therein.

GDNF and NT-3 are regarded as preferred candidates for initial testing for use in treating motor neurons as disclosed herein, since they appear to have relatively strong activity and effects (compared to other known neurotrophic factors) when administered to motor neurons in particular, as indicated by tests done in the prior art.

Numerous gene promoters that drive gene expression in motor neurons are known and available, and can be used in tests such as disclosed herein. One class of promoters that may deserve particular attention for use to transfect spinal motor neurons include the promoters that drive expression of the so-called alpha-i subunits of glycine receptors, in spinal motor neurons. Since glycine receptors are not present at high concentrations in nocioceptive or other sensory neurons, it is anticipated that use of one or more types of promoters derived from genes which express one or more subunits of such glycine receptors (or other genes that are expressed more actively in spinal motor neurons than in sensory neurons) may help increase selective expression of vector-borne neurotrophic genes in the desired target neurons, while minimizing potential adverse side effects that might be caused by unwanted expression in untargeted cells.

Another class of promoter that might be useful in some situations involving spinal motor neuron transfection drives the expression of a protein known as the polio virus receptor; this receptor protein is not present at substantial levels in sensory neurons, so the promoter is presumed to be silent in sensory neurons. However, it should be noted that many types of lab animals (including mice and rats) do not have polio virus receptor genes or promoters; therefore, it likely would be somewhat more difficult to carry out various types of research in animal models, if polio virus promoters are used.

Alternately, various types of viral and other promoters which are known to be unusually strong promoters in mammalian cells can be used if desired, for purposes such as inducing the highest possible levels of expression of NGF (or other CNS-active polypeptides) in transfected cells. Examples of such strong promoters include the early gene promoter from cytomegalovirus, and the late gene promoter from simian virus-40. Inducible gene promoters can also be used if desired, so long as the inducing factor which activates the selected promoter can be administered in a way which ensures that it will be transported into transfected neurons in adequate quantities.

Accordingly, adenoviral vectors carrying neurotrophic genes that function in motor neurons, such as GDNF or NT-3, can be assembled, using various components and methods as disclosed in the articles cited above.

If desired, an “epitopic tag” sequence can be incorporated into the coding region of the vector gene, to facilitate detection and monitoring of the polypeptide encoded by the vector gene in various tissues of test animals. This approach is discussed in more detail in Example 1, and in articles such as Moller et al 1998. If such an approach is used, it is important to confirm that the polypeptide expressed by the vector construct is able to undergo all steps that are necessary for proper delivery to the neurons that are being targeted for treatment by that polypeptide, using methods described in articles such as Altar & Bakhit 1991, Ferguson et al 1991, DiStephano et al 1992, and von Bartheld 2000.

Adenovirus vectors carrying the desired gene construct can be administered to spinal motor neurons via intramuscular injection, into a lower limb, of adenovirus vector suspended in a volume of solution compatible with adenovirus and tissue vigor (such as physiological saline solution). Methods for propagating, purifying, concentrating, and titering solutions containing adenoviral vectors can be found in publications such as the chapter by Engelhardt (pp. 169-184) in Methods in Molecular Medicine: Gene Therapy Protocols (P. Robbins, ed., 1997), and Haase et al 1998 provides information on dosages and administration techniques for efficient administration of adenovirus vectors via intramuscular injection, for transfer of genes into spinal motor neurons in laboratory rats. If desired, electromyographic injection procedures can be used to help ensure that the fluid is injected into the exact desired location.

### Example 15

### Monitoring Vector Genes and Polypeptides in Transfected Motor Neurons, Upper Motor Neurons, and Brain Tissue

Tests that use laboratory animals should be designed in a way that will simplify the tasks involved in (i) measuring and monitoring the movement, locations, and concentrations of the genetic vectors and the vector-encoded polypeptides in various cells and spinal cord regions that are likely to be contacted and affected by the polypeptides; and (ii) obtaining reliable and useful statistical data which accurately reflect such results. The necessary tasks can be simplified and rendered more reliable by administering the gene vector to the muscles on one side of a test animal (such as injecting the vector solution into a hind leg), and treating the other side of the same animal as a control, by using a control vector which carries, for example, an innocuous and/or non-functional gene, a nonsense DNA sequence which does not encode any polypeptide, or a marker gene which encodes a polypeptide that can be easily detected if expressed in mammalian cells, but which has no significant physiological effect. Upon subsequent histological processing, the left and right regions of the spinal cord (and the brain, if desired) can be compared against each other, to assess the movement, concentrations, and effects of the vector DNA and/or the polypeptide(s) encoded by the vector gene(s).

After allowing sufficient time for gene expression (24 to 72 hours), the effectiveness of gene vector delivery can be assessed by removing the lumbar spinal cord from some experimental animals and processing the tissue to monitor for expression of the neurotrophic factor gene within the spinal motor neurons. These types of analyses can use methods such as: (i) hybridization of cellular mRNA with DNA probes that are complementary to the gene vector mRNA, but not to endogenous mRNA sequences, using procedures as described in articles such as Xian and Zhou 2000; (ii) techniques which use “polymerase chain reaction” (PCR) reagents and methods to detect DNA or mRNA sequences from the viral vector, as described in articles such as Chie et al 2000; and, (iii) immunostaining, ELISA, or similar methods which use antibodies that selectively bind to the vector polypeptide but not to the endogenous polypeptide in the test species. Many such antibody preparations are commercially available; alternately, if desired (such as to detect polypeptides having a specific “epitopic tag”), they can be prepared using methods disclosed in articles such as Conner 2000, Rush et al 2000, and Zhang et al 2000.

The goal of the procedures outlined in Examples 14 and 15 is to use transfected spinal motor neurons which straddle the blood-brain barrier to deliver therapeutic polypeptides to the upper motor neurons that lie protected wholly within the BBB. This is achieved by: (i) creating vector-encoded neurotrophic polypeptides, by expressing the vector-borne neurotrophin genes, and then, (ii) transporting and delivering those polypeptides to locations in the spinal cord which are protected from foreign polypeptides by the BBB, but which are accessible to axonal processes of upper motor neurons.

To facilitate the process of identifying the exact regions within the spinal cord where such polypeptides are most likely to be delivered by transfected spinal motor neurons, “transneuronal labelling” studies can be carried out, using appropriate tracer procedures and reagents. Such studies which made use of the pseudorabies virus are described in articles such as Card et al 1990.

Transneuronal labelling can also be used to obtain evidence of delivery of polypeptides such as NT-3 or GNDF to the upper motor neurons resulting in upper motor neuron sprouting and establishment of synaptic connections with the transfected BBB straddling lower motor neuron. The number of transneuronally labelled upper motor neurons in the brain and brainstem will be greater by virtue of an increased strength and number of synaptic contacts between upper and lower motor neurons.

Where a control and test vector are administered to opposite sides of the experimental animal, appropriate differences between the left and right sides of the spinal cord will be observed. Severing the descending axonal tracts in some animals can be used to confirm that the NT-3 or GDNF detected in the brain in other experimental animals was derived from retrograde transport from transfected lower motor neurons.

### Example 16

### Monitoring Physiological and Muscular Effects Caused by Delivering Neurotrophic Factors to Upper Motor Neurons

The physiological effects of administering neurotrophic factors such as NT-3 or GDNF into the spinal cord or brain, using genetic vectors as disclosed herein, include effects on the neuroanatomy of the spinal cord and brain, and effects on the physiology and behavior of the animal. In particular, improvements and benefits in muscle strength, muscle control, and muscle tone that can be provided by using the genetic vectors disclosed herein can be evaluated using various methods.

These methods require a basic understanding of what has previously been seen in various prior tests using animal models. It must also be recognized that most such tests are necessarily carried out by first inflicting some sort of neuronal damage or injury upon the spinal cord or motor neuron system of an animal, then waiting for some period of time for the injury to be more fully manifested, then administering some sort of test treatment (such as direct infusion of a neurotrophic factor into the spinal fluid of the animal, using a hypodermic needle), and finally, by subsequent testing of the animal to determine whether the test treatment was able to reduce the extent of damage that was inflicted by that same type of injury in control animals or limbs. As summarized in Example 4, comparative tests usually involve either or both of the following: (i) different control animals, or (ii) treatment of the two different sides of the same animal in different manners.

It should be recognized that many such tests use “axotomy”, which refers to surgically severing the axon. As noted in Example 4, the axon is crucial to a neuron's functioning, and over a span of time measured in days, if a neuron's axon has been severed in a location that is relatively close to the neuron's cell body, the neuron likely will begin to atrophy and will eventually die, even though the entire remainder of the neuron is undamaged. The reasons for this are complex, and are generally believed to involve cellular factors (and especially neurotrophic factors) involved in nervous system development. According to the so-called “neurotrophic hypothesis”, a developing brain in a fetus initially generates an oversupply of neurons, then a pruning process begins. During the pruning stage, neurons that do not actively continue to receive incoming nerve signals (and/or are not contacted by one or more types of neurotrophic factors) die off, in a form of programmed cell death called “apoptosis.”

Many animal studies have demonstrated that application of various neurotrophic factors (usually by injection into spinal fluid or spinal tissue) can prevent the type of atrophy, degeneration, and death that can be induced in upper motor neurons by axotomy injury. Examples of such animal tests, and the results that arise when neurotrophic factors are applied to such neurons, are reported in articles such as Novikova et al 2000, Giehl and Tetzlaff 1996, and Giehl et al 1997.

However, where the axotomizing injury is distant from lower motor neurons that have been genetically transfected with foreign genes (as will occur if and when the genetic vectors disclosed herein are used in the manner disclosed in Example 14), it will not be easy to demonstrate that the vectors and genes of this invention had an effect on axotomy-induced atrophy or degeneration, because the axotomy injury will interfere with the normal retrograde axonal flow of neurotrophic factor within the transfected peripheral motor neuron. If the transfected lower motor neurons lie within a few millimeters of the severed end of the upper motor neuron, the upper motor neuron is more likely to have access to the neurotrophic polypeptide that is expressed and secreted by transfected motor neurons. Accordingly, appropriate experimental designs which use axotomy injuries preferably should use an axotomic locations that are within reasonably close distances to the closest tips of the motor neurons that can reasonably be transfected by using muscle injections.

Administration of neurotrophic factors has also been shown, in published reports, to stimulate sprouting and regeneration of injured axons (e.g., Schnell et al 1994). Accordingly, anterograde tract tracing studies, involving injection of tracer compounds such as biotinylated dextran into the motor cortex (as described in Ferguson et al 2001) may be used to study and demonstrate various effects (such as stimulation of sprouting and regeneration) when genetic vectors such as disclosed herein are used to deliver factors such as NT-3 or GDNF via lower motor neurons that lie within about 1 to 5 mm of the severed end of an injured upper motor neuron.

Use of the invention to deliver NT-3 or GDNF via lower motor neurons is also expected to establish a chemoattractant gradient, where the highest concentrations of the attractant compound are likely to be centered near the transfected lower motor neurons (unless such molecules are rapidly cleared or dispersed, such as by active uptake into other cells). Regenerating injured motor neurons typically will respond to this type of chemoattractant gradient, by growing in the direction of increasing concentrations of the chemoattractant. By making use of anterograde tract tracing methods, such as described in Ferguson et al 2001, the influence of such chemoattractant gradient on the directional growth of the regenerating injured upper motor neurons may be evaluated and demonstrated.

Establishment of a chemoattractant gradient emerging from transfected lower motor neurons is also likely to accelerate the rate at which regenerating injured upper motor neurons will form new synaptic connections with the neurons that are releasing a chemoattractant compound, such as GDNF or NT-3. This type of acceleration in the rate of synaptic connection can be demonstrated by undertaking time-course studies in animal models, and monitoring the time course of appearance of a transneuronal tracer compound in the upper motor neuron, if the tracer was injected into regions outside the BBB. Suitable transneuronal tracer methods are described in articles such as Ugolini 1995, Travers et al 1995, and Card et al 1990.

Establishment of functional synaptic connections between upper and lower motor neurons also will alter the electrical behavior of the lower motor neurons. The primary effect of upper motor neuron activity tends to be inhibitory: therefore, when this form of inhibition is lost, lower motor neurons tend to enter a hyperactive status. Accordingly, restored or regenerated connections with upper motor neurons is expected to reestablish more normal levels of activity in the lower motor neurons, which will manifest as more normal sensory reflexes.

Electrophysiological methods can be used to monitor the time course of such changes, and can provide an indicator of whether functional synaptic reconnections are being established between upper and lower motor neurons.

Establishment of functional reconnection between upper and lower motor neurons will also result in observable changes in motor function, including muscle strength, muscle tone, and coordination. The time course of change in muscle strength, and the ability of an animal to perform fine motor or coordinated motor tasks (such as by overcoming challenges in order to obtain food), can be tested and monitored by methods described in the literature and known to those skilled in the art.

### Example 17

### Use of Lentivirus-Derived Vectors for Transfecting Spinal Motor Neurons that will Transport Neurotrophic Polypeptides to Upper Motor Neurons

The methods described in articles such as Hottinger et al 2000 can be used to construct a lentivirus vector capable of carrying a gene construct which has (i) a GDNF, NT-3, or other neurotrophic polypeptide coding sequence, and (ii) a gene promoter suited for gene expression in transfected spinal motor neurons, as described in Example 14. This type of lentivirus-derived gene vector may be administered to the accessible projections of spinal motor neurons via intramuscular injection, using procedures such as described in Example 14, above.

After transfection of the spinal motor neurons, expression of the encoded polypeptide in the transfected neurons, and delivery of the polypeptide into spinal cord or brain tissue protected by the BBB, can be measured and monitored by methods such as described in Example 15, above. Muscular and other physiological effects of the neurotrophic polypeptide, and the ability of the neurotrophic polypeptide to prolong neuronal survival following axotomy or similar challenge, can be measured and monitored by methods such as described in Example 16, above.

### Example 18

### Preparation and use of Liposome Vectors for Transfecting Spinal Motor Neurons that will Transport Neurotrophic Polypeptides to Upper Motor Neurons

As noted in Example 6, methods for preparing DNA plasmid-lipid complexes for transfecting mammalian cells are described in publications such as the chapter by Nabel (pp. 127-133) in Methods in Molecular Medicine: Gene Therapy Protocols (P. Robbins, ed., 1997), and can be adapted for cell types or specialized uses by routine testing of various preparative mixtures.

Liposomal vectors carrying neurotrophic factor genes may be administered to the accessible projections of spinal motor neurons via intramuscular injection, using procedures such as described in Example 14, above. After transfection of the spinal motor neurons, expression of the encoded polypeptide in the transfected neurons, and delivery of the polypeptide into spinal cord or brain tissue protected by the BBB, can be measured and monitored by methods such as described in Example 15, above. Muscular and other physiological effects of the neurotrophic polypeptide, and the ability of the neurotrophic polypeptide to prolong neuronal survival following axotomy or similar challenge, can be measured and monitored by methods such as described in Example 16, above.

### Example 19

### Construction and use DNA Vectors that Target Endocytotic Receptors on Spinal Motor Neurons that will Transport Neurotrophic Polypeptides to Upper Motor Neurons

Reagents and procedures for preparing genetic vectors using ligands which bind to endocytotic receptors on neurons are discussed in Example 7, above. Those procedures include methods for using monoclonal antibodies, or repeated selection cycles involving, phage display libraries, to identify and create high-affinity ligands that will actively bind to endocytotic receptors on spinal motor neurons.

As introduced in Example 7, ligands to the p75 receptor can be used to target gene delivery to the spinal motor neurons. While spinal motor neurons normally express only low levels of p75, these spinal motor neurons upregulate their expression of p75 in response to injury or deprivation of neurotrophic factors, and in various diseases such as amyotrophic lateral sclerosis. Therefore, a p75 targeting gene vector enables enhanced or targeted delivery of genes encoding therapeutic proteins to spinal motor neurons in need of therapeutic support.

The efficiency of gene vectors which target spinal motor neurons can be enhanced by including, in the vector construct itself or in an injectable solution which contains the gene construct, a compound that can concentrate the gene vector at the “motor endplate” in an injected muscle. For this purpose, botulinum toxin may be, useful; alternately, a monoclonal antibody may be generated, using a 17-amino acid peptide sequence from the a, subunit of the acetylcholine receptor as the antigen (see Yoshikawa et al 1997). In a disease called myasthenia gravis, a patient develops antibodies against this epitope of the acetylcholine receptor, and these antibodies are localized on the muscle endplate in the synaptic cleft.

Accordingly, “receptor-targeting” gene vectors which use such receptor-binding ligands and possibly other enhancements can be used to carry gene constructs that have a GDNF, NT-3, or other neurotrophic polypeptide coding sequence, and a gene promoter suited for gene expression in transfected spinal motor neurons, as described in Example, 14. Administration to spinal motor neurons via intramuscular injection of ligand-DNA complexes can use procedures such as described in Example 14, above. After transfection of the spinal motor neurons, expression of the encoded polypeptide in the transfected neurons, and delivery of the polypeptide into spinal cord or brain tissue protected by the BBB, can be measured and monitored by methods such as described in Example 15, above. Muscular and other physiological effects of the neurotrophic polypeptide, and the ability of the neurotrophic polypeptide to prolong neuronal survival following axotomy or similar challenge, can be measured and monitored by methods such as described in Example 16, above.

### Example 20

### Transfection of Spinal Motor Neurons using Trans-Neuronal Vectors to Deliver Neurotrophic Factor Genes to CNS Neurons in Contact with the Spinal Motor Neurons

Genetic vectors that may have “transneuronal” transport capability are described in Example 8, above. Such vectors designed for use in transfecting spinal motor neurons can be assembled using methods such as described in Example 8, and can carry gene constructs such as described in Example 14, above. Administration to spinal motor neurons via intramuscular injection can use procedures such as described in Example 14, and post-transfection monitoring can use methods such as described in Examples 15 and 16.

### Example 21

### Delivery of Neurotrophic Factors into the Brainstem, by Injection of Vectors into the tongue to Transfect Lower Motor Neurons of the Hypoglossal Nucleus

Genetic vectors derived from adenoviruses, herpes viruses, or lentiviruses, or using cationic liposomes, ligands that bind to endocytotic receptors, and/or trans-neuronal polypeptides, can be created as described in the examples above. These types of vectors can carry neurotrophic gene constructs (or, if desired, gene constructs that express recombinant antibodies or similar polypeptides that inhibit one or more neurite inhibitory molecules such as IN1 or No-Go) which will be expressed in transfected motor neurons, as described in the foregoing examples.

As illustrated in FIG. 6, such vectors can be used to transfect a certain class of lower motor neurons which have projections that are accessible outside the BBB, in the tongue. These neurons, known as “motor neurons of the hypoglossal nucleus,” are synaptically connected to other neurons in certain regions in the brainstem. Therefore, the motor neuron terminals inside the tongue offer a relatively direct passageway for delivering neurotrophic polypeptides into neurons of the brainstem that synapse with or have projections that lie near these transfected lower motor neurons.

Administration to these motor neurons can be achieved via injection into the tongue, using general procedures such as described in Example 14 but adapted for injection into the muscles of the tongue. Post-transfection monitoring in test animals can use general methods such as described in Examples 15 and 16, appropriately adapted to monitor for delivery of exogenous peptides (such as epitope-tagged NGF, NT-3, or GDNF) to neurons in the brain stem, as well as other methods known to those skilled in the art. As one example, for use in animals, various transneuronal tracer methods, such as described by Ugolini 1995, Travers et al 1995, or Card et al 1990, can be used to label neurons in brainstem which make contact with transfected lower motor neurons in the hypoglossal nucleus. By counting the number of second-order neurons which contact the hypoglossal nucleus in animals treated with the invention, and comparing such data to numbers seen in control animals, evidence can be obtained of the delivery of neurotrophic factor to second-order neurons in the brainstem.

As another example of a monitoring method which may be useful in human patients, hyperexcitable reflex blinks are a symptom of Parkinson's disease, which correlates clinically with the severity of the disease. Tonically active serotonergic “raphe neurons” normally inhibit the spinal trigeminal neurons involved in reflex blink circuits, and deterioration of these serotonergic raphe neurons leads to the hyperexcitable blink reflex (e.g., Basso and Evinger 1996). Accordingly, evidence of the effectiveness of clinical use of the invention to reduce the rate of degeneration of serotonergic neurons of the brainstem can be obtained by monitoring the hyperexcitable blink reflex in patients suffering Parkinson's disease.

Another of method for monitoring preservation of brainstem neurons in human patients with Parkinson's disease involves taking measurements of a patient's swallowing reflex. The swallowing reflex involves a coordinated movement of tongue and other oral and respiratory muscles and, in Parkinson's disease, the time between stimulus and reflex swallowing is abnormally prolonged. A swallowing reflex test involves delivery of a small volume of water into the throat, via intranasal catheter, as the swallowing reflex stimulus. The initiation of swallowing reflex can be accurately measured using surface electrode electromyographic recording of the muscles in the throat and used to calculate the time between stimulus and reflex swallowing (Iwasaki et al 2000). Accordingly, evidence of the effectiveness of clinical use of the invention to reduce the rate of degeneration of neurons of the brainstem can be obtained by monitoring the swallowing reflex in patients suffering Parkinson's disease.

Thus, there has been shown and described a new and useful means for non-invasive transport of therapeutic or other useful polypeptides through the BBB, into brain and spinal tissue (and in particular to neurons that lie wholly within the BBB). Although this invention has been exemplified for purposes of illustration and description by reference to certain specific embodiments, it will be apparent to those skilled in the art that various modifications, alterations, and equivalents of the illustrated examples are possible. Any such changes which derive directly from the teachings herein, and which do not depart from the spirit and scope of the invention, are deemed to be covered by this invention.

### REFERENCES

Abbott, N. J., et al, “Transporting therapeutics across the blood-brain barrier,” Mol. Med. Today 2(3): 106-13 (1996)

Altar, C. A., et al, “Receptor-mediated transport of human recombinant nerve growth factor from olfactory bulb to forebrain cholinergic nuclei,” Brain Res. 541: 82-8 (1991)

Banks, W. A., et al, “Passage of peptides across the blood-brain barrier: pathophysiological perspectives,” Life Sci. 59(23): 1923-43 (1996)

Banks, W. A., “Physiology and pathology of the blood-brain barrier: implications for microbial pathogenesis, drug delivery and neurodegenerative disorders,” J. Neurovirol. 5(6): 538-55 (1999)

Barbacid, M., “Neurotrophic factors and their receptors,” Curr. Opin. Cell Biol. 7: 148-155 (1995)

Barde, Y. A. “The nerve growth factor family,” Prog. Growth Factor Res. 2: 237-248 (1991)

Bartlett, J. S., et al, “Methods for the construction and propagation of recombinant adeno-associated virus vectors,” pp. 25-40 in Robbins 1997

Barnett, E. M., et al, “Two neurotropic viruses, herpes simplex virus Type 1 and mouse hepatitis virus, spread along different neural pathways from the main olfactory bulb,” Neurosci. 57: 1007-1025 (1993)

Baumgartner, B. J., et al, “Neuroprotection of spinal motor neurons following targeted transduction with an adenovirus vector carrying the gene for glial cell-derived neurotrophic factor, ” Exp. Neurol. 153: 102-112 (1998)

Becerril, B., et al, “Towards selection of internalizing antibodies from phage libraries,” Biochem. Biophys. Res. Comm. 255: 386-393 (1999)

Begley, D. J., “The blood-brain barrier: principles for targeting peptides and drugs to the central nervous system,” J. Pharm. Pharmacol. 48(2): 136-46 (1996)

Bennet, D. L., et al, “NGF but not NT-3 or BDNF prevents the A fiber sprouting into lamina II of the spinal cord that occurs following axotomy,” Mol. Cell. Neurosci. 8: 211-220 (1996)

Blesch, A., et al,“Ex vivo gene therapy for Alzheimer's disease and spinal cord injury,” Clin. Neurosci. 3: 268-274 (1996)

Blessing, W. W., et al, “Transneuronal labelling of CNS neurons with herpes simplex virus,” Prog. Neurobiol. 44: 37-53 (1994)

Bolivar, F., et al, “Construction and characterization of new cloning vehicles: A multipurpose cloning system,” Gene 2: 95-113 (1977)

Bonate, P. L., “Animal models for studying transport across the blood-brain barrier,” J. Neurosci. Methods 56(1): 1-15 (1995)

Bothwell, M., “Functional interactions of neuurotrophins and neuurotrophin receptors,” Ann. Rev. Neurosci. 18: 223-253 (1995)

Card, J. P., et al, “Neurotropic properties of pseudorabies virus: Uptake and transneuronal passage in the rat central nervous system,” J. Neurosci. 10: 1974-1994 (1990)

Causing, C. G., et al, “Construction of transgenic animals overproducing neurotrophins and their receptors,” Methods in Molecular Biology 169: 167-191 (2000)

Chie, E., et al, “Quantification of neurotrophin mRNA by RT-PCR,” Methods in Molecular Biology 169: 81-90 (2000)

Choi-Lundberg, D. L., et al, “Dopaminergic neurons protected from degeneration by GDNF gene therapy,” Science 275: 838-841 (1997)

Christensen, M. D., et al, “Anti-NGF prevents thermal allodynia in chronic central pain following spinal cord injury,” Soc. Neurosci. Abstr. #208.4 (1996)

Christensen, M. D., et al, “Spinal cord injury and anti-NGF treatment results in changes in CGRP density and distribution in the dorsal horn of the rat,” Exp. Neurol. 147: 463-475 (1997)

Cloughesy, T. F., et al, “Pharmacological blood-brain barrier modification for selective drug delivery,” J. Neuroncol. 26(2): 125-32 (1995)

Conner, J. M., et al, “The localisation of nerve growth factor-like immunoreactivity in the adult rat basal forebrain and hippocampal formation,” J. Comp. Neurol. 319: 454-462 (1992)

Conner, J. M., “Localization of neurotrophin proteins within the central nervous system by immunohistochemistry,” Methods in Molecular Biology, Vol 169: 3-20 (2000)

Cuello, A. C., et al, “Trophic effects on cholinergic innervation in the cerebral cortex of the adult rat brain,” Mol. Neurobiol. 6: 451-461 (1992)

Cunningham, A. M., et al, “Olfactory receptor neurons exist as distinct subclasses of immature and mature cells in primary culture,” Neurosci. 93: 1301-1312 (1999)

Curiel, D. T., “Methods for gene transfer using DNA-Adenovirus conjugates,” Methods in Molecular Medicine: 25-40 (1997)

Davis, T. P., et al, “Conformationally constrained peptide drugs targeted at the blood-brain barrier,” NIDA Res. Monogr. 154(1): 47-60 (1995)

de Boer, A. G., et al, “The transference of results between blood-brain barrier cell culture systems,” Eur. J. Pharm. Sci. 8(1): 1-4 (1999)

Deng, Y. S., et al, “Effects of endogenous neurotrophins on sympathetic sprouting in the dorsal root ganglia and allodynia following spinal nerve injury,” Exp. Neurol. 164: 344-350 (2000)

Dijkhuizen, P. A., et al, “Adenoviral vector-directed expression of neurotrophin-3 in rat dorsal root ganglion explants results in a robust neurite outgrowth response,” J Neurobiol. 33: 172-184 (1997)

DiStephano, P. S., et al, “The neurotrophins BDNF, NT-3 and NGF display distinct patterns of retrograde transport in peripheral and central neurons,” Neuron 8: 983-993 (1992)

Engelhardt, J. F., “Methods for adenovirus-mediated gene transfer to airway epithelium,” pp. 169-184 in Robbins 1997

Federoff, H. J., et al, “Expression of nerve growth factor in vivo from a defective herpes simplex virus 1 vector prevents effects of axotomy on sympathetic ganglia,” Proc. Natl. Acad. Sci. USA 89: 1636-1640 (1992)

Ferguson, I. A., “Basic fibroblast growth factor: Receptor-mediated internalization, metabolism, and anterograde axonal transport in retinal ganglion cells,” J. Neurosci. 10: 2176-2189 (1990)

Ferguson, I. A., “Receptor-mediated retrograde transport in CNS neurons following intraventricular administration of NGF and growth factors,” J. Comp. Neurol. 313: 680-692 (1991)

Fiers, W., et al, “Complete nucleotide sequence of SV40 DNA,” Nature 273: 113-20 (1978)

Findeis, M. A., et al, “Methods for targeted gene transfer to liver using DNA-protein complexes,” pp. 135-152 in Robbins 1997

Fischer, W., et al, “Amelioration of cholinergic neuron atrophy and spatial memory impairment in aged rats by nerve growth factor,” Nature 329: 65-68 (1987)

Fischer, W., et al, “NGF improves spatial memory in aged rats as a function of age,” J. Neurosci. 11: 1889-1906 (1991)

Fisher, L. J., “In vivo and ex vivo gene transfer to the brain,” Curr. Opin. Neurobiol. 4: 735-41 (1994)

Funakoshi, H., et al, “Targeted expression of a multifunctional chimeric neurotrophin in the lesioned sciatic nerve accelerates regeneration of sensory and motor axons,” Proc. Natl. Acad. Sci. USA 95: 5269-5274 (1998)

Garofalo, L., et al, “Nerve growth factor-induced synaptogenesis and hypertrophy of cortical cholinergic terminals,” Proc. Natl. Acad. Sci. USA 89: 2639-2643 (1992)

Garofalo, L., et al, “Nerve growth factor and the monsialoganglioside GM1,” Exp. Neurol. 125: 195-217 (1994)

Gavel, C., et al, “Adenoviral gene transfer of ciliary neurotrophic factor and brain-derived neurotrophic factor leads to long-term survival of axotomized motor neurons,” Nat. Med. Jul;3(7): 765-70 (1997)

Giehl, K. M., et al, “BDNF and NT-3, but not NGF, prevent axotomy induced death of rat corticospinal neurons in vivo,” Eur. J. Neurosci. 8: 1167-1175 (1996)

Giehl, K. M., et al, “GDNF is a trophic factor for adult rat corticospinal neurons and promotes their long-term survival after axotomy in vivo,” Eur. J. Neurosci. 9: 2479-2499 (1997)

Goins, W. F., et al, “Herpes simplex virus type 1 vector-mediated expression of nerve growth factor protects dorsal root ganglion neurons from peroxidase toxicity,” J. Virol. 73: 519-532 (1999)

Goldstein, G. W., et al, “The blood-brain barrier,” Sci. Am. 255: 74-83 (1986)

Graham, F. L., et al, “Methods for construction of adenoviral vectors,” Mol. Biotechnol. 3: 207-220 (1995)

Granholm, A. C., et al, “A non-invasive system for delivering neural growth factors across the blood-brain barrier: a review,” Rev. Neurosci. 9(1): 31-55 (1998)

Guegan, C., et al, “Reduction in cortical infarction and impairment of apoptosis in NGF-transgenic mice subjected to permanent focal ischemia,” Brain Res. Mol. Brain Res. 55: 133-140 (1998)

Hart, S. L., et al, “Cell binding and internalization by filamentous phage displaying a cyclic Arg-Gly-Asp-containing peptide,” J. Biol. Chem. 269: 12468-12474 (1994)

Hefti, F., “Neurotrophic factor therapy for nervous system degenerative diseases,” J. Neurobiol. 25: 1418-1435 (1994)

Holtmaat, A. J. G. D., et al, “Efficient adenoviral vector-directed expression of a foreign gene to neurons and sustentacular cells in the mouse olfactory neuroepithelium,” Mol. Brain Res. 41: 148-156 (1996)

Horowitz, L. F., “A genetic approach to trace neural circuits,” Proc Natl. Acad. Sci. 96: 3194-3149 (1999)

Hottinger, A. F., et al, “Complete and long-term rescue of lesioned adult motoneurons by lentiviral-mediated expression of glial cell line-derived neurotrophic factor in the facial nucleus,” J. Neurosci. 20: 5587-5593 (2000)

Iwasaki, K., et al, “The effects of the traditional Chinese medicine “Banxia Houpe Tang (Hange-Koboku To)” on the swallowing reflex in Parkinson's disease,” Phytomedicine 7: 259-263 (2000)

Janes, K. A., et al, “Polysaccharide colloidal particles as delivery systems for macromolecules,” Adv. Drug Deliv. Rev. 23: 83-97 (2001)

Kaplitt, M. G., et al, “Defective viral vectors as agents for gene transfer in the nervous system,” J. Neurosci. Methods 71: 125-132 (1997)

Karpati, G., et al, “The principles of gene therapy for the nervous system,” Trends Neurosci. 19: 49-54 (1996)

Kastin, A. J., et al, “Peptides crossing the blood-brain barrier: some unusual observations,” Brain Res. 27 848(1-2): 96-100 (1999)

Kniesel, U., et al, “Tight junctions of the blood-brain barrier,” Cell. Mol. Neurobiol. 20(1): 57-76 (2000)

Knight, A., et al, “Non-viral neuronal gene delivery mediated by the HC fragment of tetanus toxin,” Eur. J. Biochem. 259: 762-769 (1999)

Krenz, N. R., et al, “Neutralizing intraspinal nerve growth factor blocks autonomic dysreflexia caused by spinal cord injury,” J. Neurosci. 19: 7405-7414 (1999)

Krenz, N. R., et al, “Nerve growth factor in glia and inflammatory cells of the injured rat spinal cord,” J. Neurochem. 74: 730-739 (2000)

Kroll, R. A., et al, “Outwitting the blood-brain barrier for therapeutic purposes: osmotic opening and other means,” Neurosurgery 42(5): 1083-99; discussion 1099-100 (1998)

Lachmann, R. H., et al, “Utilization of the herpes simplex virus type 1 latency-associated regulatory region to drive stable reporter gene expression in the nervous system,” J. Virol. 71: 3197-3207 (1997)

Lafay, F., et al, “Spread of the CVS strain of rabies virus and of the avirulent mutant AvO1 along the olfactory pathways of the mouse after intranasal inoculation,” Virol. 183: 320-330 (1991).

La Salle, G., et al, “An adenovirus vector for gene transfer into neurons and glia in the brain,” Science 259: 988-990 (1993)

Langer R., “New methods of drug delivery,” Science 249: 1527-33 (1990)

Langer, R., et al, “New directions in CNS drug delivery,” Neurobiol. Aging 10: 642-4 (1989)

Lewin, G. R., et al, “Physiology of the neurotrophins,” Ann. Rev. Neurosci. 19: 289-317 (1996)

Li, S., et al, “Nonviral gene therapy: Promises and challenges,” Gene Therapy 7: 31-34 (2000)

Lim, S. T., et al, “Preparation and evaluation of the in vitro drug release properties and mucoadhesion of novel microspheres of hyaluronic acid and chitosan,” J. Control Release 66: 281-92 (2000)

Lindsay, R. M., “Neurotrophins and receptors,” Prog. Brain Res. 103: 3-14 (1994)

Lindsay, R. M., et al, “GDNF in a bind with known orphan: Accessory implicated in a new twist,” Neuron 17: 571-574 (1996)

Lo, D. C., “Neurotrophic factors and synaptic plasticity,” Neuron 15: 979-981 (1995)

Madrid, Y., et al, “New directions in the delivery of drugs and other substances to the central nervous system,” Adv. Pharmacol. 22: 299-324 (1991)

Martinez-Fong, D., et al, “Neurotensin-SPDP-poly-L-lysine conjugate: a non-viral vector for targeted gene delivery to neural cells,” Brain Res. Mol. Brain Res. 69: 249-262 (1999)

McMahon, S. B., et al, “Peripheral neuropathies and neurotrophic factors: Animal models and clinical perspectives,” Curr. Opin. Neurobiol. 5: 616-624 (1995)

Moller, J. C., et al, “Subcellular localisation of epitope-tagged neurotrophins in neuroendocrine cells,” J. Neurosci. Res. 51: 463-472 (1998)

Nabel, E. G., “Methods for liposome-mediated gene transfer to the arterial wall,” pp. 127-133 in Robbins 1997

Novikova, L., et al, “Survival effects of BDNF and NT-3 on axotomised rubrospinal neurons depend on the temporal pattern of neurotrophin administration,” Eur. J. Neurosci. 12: 776-780 (2000)

Ozawa, C. R., et al, “Neuroprotective potential of a viral vector system induced by a neurological insult,” Proc. Natl. Acad. Sci. USA 97: 9270-9275 (2000)

Pardridge, W. M., “Blood-brain barrier biology and methodology,” J. Neurovirol. 5(6): 556-69 (1999)

Pardridge, W. M., “CNS drug design based on principles of blood-brain barrier transport,” J. Neurochem. 70(5): 1781-92 (1998)

Phelps, C. J., et al, “Pituitary hormones as neurotrophic signals,” Proc. Soc. Exp. Biol. Med. 222: 39-58 (1999)

Pickard, G. E., et al, “Intravitreal injection of the attenuated psuedorabies virus PRV Bartha results in infection of the hamster suprachiasmatic nucleus only by retrograde transsynaptic transport via autonomic circuits,” J Neurosci 22: 2701-2710 (2002)

Price, D. L., et al, “Tetanus toxin: direct evidence for retrograde intraaxonal transport,” Science 188: 945-947 (1975)

Qasba, P., et al, “Tissue and zonal-specific expression of an olfactory receptor transgene,” J. Neurosci. 18: 227-236 (1998)

Rapoport, S. I., “Osmotic opening of the blood-brain barrier: principles, mechanism, and therapeutic applications,” Cell. Mol. Neurobiol. 20: 217-30 (2000)

Rillosi, M., et al, “Modelling mucoadhesion by use of surface energy terms obtained from the Lewis acid-Lewis base approach. II. Studies on anionic, cationic, and unionisable polymers,” Pharm. Res. 12: 669-75 (1995)

Robbins, P., ed., Methods in Molecular Medicine: Gene Therapy Protocols (Humana Press, Totowa, N.J., 1997)

Rochat, B., et al, “Drug disposition and targeting. Transport across the blood-brain barrier,” Pharm. Biotechnol. 12: 181-200 (1999)

Romero, M. I., et al, “Adenoviral gene transfer into the normal and injured spinal cord,” Gene Ther. 5: 1612-161 (1997)

Romero, M., et al, “Extensive sprouting of sensory afferents and hyperalgesia induced by conditional expression of nerve growth factor in the adult spinal cord,” J. Neurosci. 20: 4435-4445 (2000)

Ruberti, F., et al, “Cloning and expression of an anti-nerve growth factor (NGF) antibody for studies using the neuroantibody approach,” Cell. Mol. Neurobiol. 13: 559-568 (1993)

Ruberti, F., et al, “The use of the RACE method to clone hybridoma cDNA when V region primers fail,” J. Immunol. Methods 173: 33-39 (1994)

Ruberti, F., et al, “Phenotypic knockout of nerve growth factor in adult transgenic mice reveals severe defects in basal forebrain cholinergic neurons, cell death in the spleen, and skeletal muscle dystrophy,” J. Neurosci. 20: 2589-2601 (2000)

Rubin, L. L., et al, “The cell biology of the blood-brain barrier,” Annu. Rev. Neurosci. 22: 11-28 (1999)

Rush, R. A., et al, “Neurotrophin immunohistochemistry in peripheral tissues,” Methods in Molecular Biology, Vol 169: 21-29 (2000)

Sahenk, Z., et al, “Gene delivery to spinal motor neurons,” Brain Res. 606: 126-129 (1993)

Sawchenko, P. E., et al, “Circuits and mechanisms governing hypothalamic responses to stress,” Prog. Brain Res. 122: 61-78 (2000)

Schacht, C. M., et al, “GDNF application into cerebrospinal fluid prevents axotomy-induced death of adult rat corticospinal neurons in vivo,” Soc. Neurosci. Abstr. #767.1 (1996)

Schatz, P. J., “Construction and screening of biological peptide libraries,” Curr. Opin. Biotech. 5: 487-494 (1994)

Schipper, N. G., et al, “Chitosans as absorption enhancers of poorly absorbable drugs. 3: Influence of mucus on absorption enhancement,” Eur. J. Pharm. Sci. 8(4): 335-43 (1999)

Schwab, M. E., et al, “Selective retrograde transsynaptic transfer of a protein, tetanus toxin, subsequent to its retrograde axonal transport,” J. Cell Bio. 82: 789-810 (1979)

Schnell, L., et al, “Neurotrophin 3 enhances sprouting of corticospinal tract during development and after adult spinal cord lesion,” Nature 367: 170-173 (1994)

Semkova, I., et al, “Neuroprotection mediated via neurotrophic factors and induction of neurotrophic factors,” Brain Res. Rev. 30: 176-188 (1999)

Servenius, B., et al, “Metastasizing neuroblastomas in mice transgenic for SV40T under the olfactory marker protein gene promoter,” Cancer Res. 54: 5198-5205 (1994)

Skaper, S. D., et al, “Neurotrophic molecules: Strategies for Designing Effective Therapeutic Molecules in Neurodegeneration,” Mol. and Cell. Neuroscience 12: 179-193 (1998)

Smith, G. M., et al, “Adenoviral-mediated gene transfer to enhance neuronal survival, growth, and regeneration,” J. Neurosci. Res. 55: 147-57 (1999)

Snider, W. D., “Functions of the neurotrophins during nervous system development,” Cell 77: 627-638 (1994)

Sofroniew, M. W., et al, “Atrophy but not death of adult septal cholinergic neurons after ablation of target capacity to produce mRNAs for NGF, BDNF, and NT-3,” J. Neurosci. 13: 5263-5276 (1993)

Staecher, H., et al, “Brain derived neurotrophic factor gene therapy prevents spiral ganglion degeneration after hair cell loss,” Otolaryngol Head Neck Surg. 119: 7-13 (1998)

Thoenen, H., et al, “Neurotrophic factors: Possibilities and limitations in the treatment of neurodegenerative disorders,” Int. Acad. Biomed. Drug Res. 7: 197-203 (1994)

Travers, J. B., et al, “Transneuronal labeling in hamster brainstem following lingual injections with herpes simplex virus-1,” Neurosci. 68: 1277-1293 (1995)

Turnbull, A. V., et al, “Regulation of the hypothalamic-pituitary-adrenal axis by cytokines,” Physiol. Rev. 79: 1-71 (1999)

Ugolini, G., “Specificity of rabies virus as a transneuronal tracer of motor networks: transfer from hypoglossal motoneurons to connected second-order and higher order central nervous system cell groups,” J. Comp. Neurol. 356: 457-480 (1995)

von Bartheld, C. S. “Tracing with radiolabeled neurotrophins,” Methods in Molecular Biology 169: 193-214 (2000)

Watson, A., et al, “A minimal CGRP gene promoter is inducible by nerve growth factor in adult dorsal root ganglion neurons but not PC12 phaeochromocytoma cells,” Eur. J. Neurosci. 7: 394-400 (1995)

Wenk, H., et al, “Cholinergic projections from magnocellular nuclei of the basal forebrain to cortical areas in rats,” Brain Res. 2: 295-316 (1980)

Wiley, R. G., et al, “Preparation of anti-neuronal immunotoxins for selective neuronal immunolesioning,” Neurosci. Protocols 93-020: 1-11 (1993)

Winter, G., et al, “Making antibodies by phage display technology,” Ann. Rev. Immunol. 12: 433-455 (1994)

Wong, E. T., et al, “Improved co-expression of multiple genes in vectors containing internal ribosome entry sites (IRESes) from human genes,” Gene Therapy 9: 337-344 (2002)

Xian, C. J., et al, “Sensitive and non-radioactive in situ detection of neurotrophin mRNAs in the nervous system,” Meth. Molec. Biol. 169: 91-96 (2000

Yan, Q., et al, “Retrograde transport of nerve growth factor (NGF) in motoneurons of developing rats: Assessment of potential neurotrophic effects,” Neuron 1: 335-343 (1998)

Yoshihara, Y., et al, “A genetic approach to visualization of multi-synaptic neuronal pathways using plant lectin transgene,” Neuron 22: 33-41 (1999)

Yuen, E. C., et al, “Therapeutic potential of neurotrophic factors for neurological disorders,” Ann. Neurol. 40: 346-354 (1996)

Zhang, S. -H., et al, “Extraction and quantification of neurotrophins,” Meth. Molec. Biol. 169: 31-41 (2000)

Zlokovic, B. V., “Cerebrovascular permeability to peptides: manipulations of transport systems at the blood-brain barrier,” Pharm. Res. 12(10): 1395-406 (1995)

TABLE 1
PARTIAL LIST OF POLYPEPTIDES THAT MAY BE DELIVERED INTO THE CNS

| | EXAMPLES |
|---|---|
| GROWTH MODULATORY FACTORS | |
| NEUROTROPHIC FACTORS | |
| And other polypeptides that stimulate growth | NGF, BDNF, NT-3, NT-4, GDNF, |
| and/or inhibit apoptosis of nerves cells including: | Neurturin, neublastin/artemin, CNTF, |
| | etc. |
| GROWTH FACTORS (GF) | |
| And other polypeptides that stimulate mitotis | Epidemal GF, Platelet derived GFs, in |
| non-neuronal cells or stem cells including: | Insulin-like GFs, Fibroblast GFs, Bone |
| | morphogenic factors |
| NEURITE INHIBITORY FACTORS | |
| And other polypeptides that provide axonal guidance | Semaphorins, ephrins, etc. |
| cues and/or inhibit nerve growth outgrowth including: | |
| CYTOKINES | |
| And other polypeptides that modulate immune system | Interleukins, Tumor necrosis factors, |
| responses including: | Interferons, LIF, TFG-B, etc. |
| PEPTIDE NEUROTRANSMITTERS | |
| And other polypeptides that modulate the transmission | Substance P, endorphins, enkephalins, |
| of electrical activity of neurons including: | NPY, VIP, CGRP, galinin, somatostatin, |
| | angiotensin, etc. |
| ENDOCRINE SYSTEM MODULATORS | |
| Hypothalamic releasing factors and other polypeptides that | GnRH, GHRH, TRH, CRF, etc. |
| stimulate release of hormones from pituitary gland including: | |
| POLYPEPTIDE HORMONES | |
| Endocrine factors | GH, FSH, LH, T3, T4, IGF-I, IGF-II, |
| | ACTH, MSH, oxytocin, inhibin, activin, |
| | etc. |
| ENZYMES | |
| Enzymes able to correct a deficiency such in a | beta-glucuronidase, arylsulfatease A |
| lysosomal storage disease | alpha-N-acetylglucosaminidase, |
| | aspartylgluosaminidase, etc. |
| ANTI-INFECTION AND OTHER AGENTS | |
| Polypeptides that can inhibit prion, virus, bacterial infections | Recombinant antibodies, |
| in the CNS, or that can neutralise target antigens expressed | receptor agonists or antagonists |
| by foreign or abnormal cells or modulate receptor function. | |

TABLE 2
EXAMPLES OF POTENTIAL THERAPIES USING
POLYPEPTIDES DELIVERED THROUGH THE BLOOD-BRAIN BARRIER

| | TARGET NEURONS | | BBB-STRADDLING |
|---|---|---|---|
| CNS DISORDERS | INSIDE BBB | POLYPEPTIDE | NEURONS |
| Alzheimer's disease, | Basal forebrain cholinergic | NGF | Olfactory receptor neurons |
| stroke | neurons | | |
| Chronic pain, | Second-order sensory | Anti-NGF | Nocioceptive sensory |
| autonomic dysreflexia | neurons in spinal cord | | neurons |
| Upper motor neuron disorders | Upper motor neurons in | NT-3 or GDNF | Spinal motor neurons |
| (ALS, etc.) | cortex, midbrain | | |
| Parkinson's disease | Brainstem neurons: raphe, | NT-3 or GDNF | Hypoglossal motor |
| | autonomic, reticular | | neurons |

1. A method for delivering neuroactive polypeptide molecules through a blood-brain barrier in a higher animal, comprising the steps of:
a. creating a genetic vector which carries at least one gene which encodes a neuroactive polypeptide, and which is constructed in a manner which will enable the genetic vector to transfect at least one class of accessible neurons which straddle the blood-brain barrier, each such accessible neuron having at least one peripheral projection that is accessible to compounds that have not penetrated the animal's blood-brain barrier, if said genetic vector is administered to such higher animal by means which cause direct contact between at least one copy of the genetic vector and at least one such peripheral projection, wherein the gene which encodes the neuroactive polypeptide is capable of expressing copies of the neuroactive polypeptide within such accessible neurons after such accessible neurons have been transfected by said genetic vector; and,
b. administering the genetic vector to the higher animal in a location and manner which ensure that: (i) at least one copy of the genetic vector contacts and transfects at least one such accessible neuron; and (ii) at least one such accessible neuron expresses the neuroactive polypeptide encoded by the gene and subsequently secretes the neuroactive polypeptide into central nervous system tissue which is protected by the blood-brain barrier.

2. The method of claim 1, wherein the accessible neurons which straddle the blood-brain barrier are sensory neurons.

3. The method of claim 2, wherein the sensory neurons are olfactory receptor neurons.

4. The method of claim 2, wherein the sensory neurons are nocioceptive neurons.

5. The method of claim 1, wherein the accessible neurons which straddle the blood-brain barrier are motor neurons.

6. The method of claim 5, wherein the motor neurons are spinal motor neurons.

7. The method of claim 5, wherein the motor neurons are motor neurons of the hypoglossal nucleus.

8. The method of claim 1, wherein the accessible neurons which straddle the blood-brain barrier are pre-ganglionic neurons of the parasympathetic nervous system.

9. The method of claim 1, wherein the accessible neurons which straddle the blood-brain barrier are pre-ganglionic neurons of the sympathetic nervous system.

10. The method of claim 1, wherein the neuroactive polypeptide is selected from the group consisting of neurotrophic factors, endocrine factors, growth factors, paracrine factors, hypothalamic releasing factors, neurotransmitter polypeptides, antibodies and antibody fragments which bind to neurotrophic factors, antibodies and antibody fragments which bind to neurotrophic factor receptors, polypeptide antagonists, agonists for a receptor expressed by a CNS cell, and polypeptides involved in lysosomal storage disease.

11. The method of claim 1, wherein the genetic vector contains cell transfection components that include at least a portion of a viral capsid active on a mammalian cell.

12. The method of claim 1, wherein the genetic vector comprises a cationic liposome or other gene transfection lipid.

13. The method of claim 1, wherein the genetic vector comprises a ligand which binds in a specific manner to at least one type of endocytotic receptor on the accessible neurons.

14. The method of claim 13, wherein the ligand has been identified through selection steps which include:
(a) subjecting a multiplicity of neurons of a selected type to contact by a phagemid or phage display library, each phage in the library carrying a candidate DNA sequence and having on an exposed phage surface a polypeptide encoded by the candidate DNA sequence, and the phage display library consisting of multiple phage particles with multiple candidate DNA sequences and corresponding polypeptides exposed on the phage surfaces;
(b) selecting at least one clonal phage line from the phage display library, wherein the selected clonal phage line was transported into neurons of the selected type.

15. The method of claim 1, wherein the genetic vector comprises a macromolecule that has been shown to promote transneuronal passage of associated genetic material out of a selected type of accessible neuron which straddles the blood-brain barrier and into at least one type of neuron which is positioned entirely within central nervous system tissue and protected by the blood-brain barrier of an animal.

16. The method of claim 1, wherein the genetic vector encodes a chimeric polypeptide comprising:
a. a leader sequence which enables or promotes anterograde transport of the chimeric polypeptide within a neuron that expresses the chimeric polypeptide; and,
b. a neuroactive sequence which can create a therapeutic benefit by reacting with cells located wholly within CNS tissue that is protected by the blood-brain barrier, after secretion within the blood-brain barrier by an accessible neuron which straddles the blood-brain barrier.

17. The method of claim 1, wherein the genetic vector encodes a chimeric polypeptide comprising:
a. a leader sequence which enables or promotes secretion of the chimeric polypeptide at neuronal termini located within CNS tissue that is protected by the blood-brain barrier of an animal; and,
b. a neuroactive sequence which can create a therapeutic benefit by reacting with cells located wholly within CNS tissue that is protected by the blood-brain barrier, after secretion within the blood-brain barrier by an accessible neuron which straddles the blood-brain barrier.

18. A method for delivering neuroactive polypeptide molecules through a blood-brain barrier in a higher animal, comprising the step of administering to the higher animal a genetic vector which carries at least one gene which encodes a neuroactive polypeptide,
wherein said genetic vector has been constructed in a manner which enables the genetic vector to transfect at least one class of accessible neuron which straddles the blood-brain barrier, if administered to such higher animal in a location and manner which causes direct contact between the genetic vector and such accessible neurons which straddle the blood-brain barrier,
and wherein said genetic vector is administered to such higher animal in a location and manner which ensures that: (i) at least one copy of the genetic vector contacts and transfects at least one such accessible neuron; (ii) at least one transfected accessible neuron expresses copies of at least one neuroactive polypeptide encoded by at least one gene carried by said genetic vector; and, (iii) at least one transfected accessible neuron secretes copies of at least one such neuroactive polypeptide, at a location within central nervous system tissue protected by the blood-brain barrier.

19. The method of claim 18, wherein the genetic vector has been designed to transfect sensory neurons.

20. The method of claim 18, wherein the genetic vector has been designed to transfect motor neurons.

21. The method of claim 18, wherein the genetic vector has been designed to transfect pre-ganglionic neurons of the parasympathetic nervous system.

22. The method of claim 18, wherein the genetic vector has been designed to transfect pre-ganglionic neurons of the sympathetic nervous system.

23. The method of claim 18, wherein the neuroactive polypeptide is selected from the group consisting of neurotrophic factors, endocrine factors, growth factors, paracrine factors, hypothalamic release factors, neurotransmitter polypeptides, antibodies and antibody fragments which bind to neurotrophic factors, antibodies and antibody fragments which bind to neurotrophic factor receptors, polypeptide antagonists, agonists for a receptor expressed by a CNS cell, and polypeptides involved in lysosomal storage disease.

24. The method of claim 18, wherein the genetic vector contains cell transfection components that include at least a portion of a viral capsid active on a mammalian cell.

25. The method of claim 18, wherein the genetic vector comprises a cationic liposome or other gene transfection lipid.

26. The method of claim 18, wherein the genetic vector comprises a ligand which binds in a specific manner to at least one type of endocytotic receptor on the accessible neurons.

27. The method of claim 18, wherein the genetic vector comprises a macromolecule that has been shown to promote transneuronal passage of associated genetic material out of a selected type of accessible neuron which straddles the blood-brain barrier and into at least one type of neuron which is positioned entirely within central nervous system tissue and protected by the blood-brain barrier of an animal.

28. The method of claim 18, wherein the genetic vector encodes a chimeric polypeptide comprising:
a. a leader sequence which enables or promotes anterograde transport of the chimeric polypeptide within a neuron that expresses the chimeric polypeptide; and,
b. a neuroactive sequence which can create a therapeutic benefit by reacting with cells located wholly within CNS tissue that is protected by the blood-brain barrier, after secretion within the blood-brain barrier by an accessible neuron which straddles the blood-brain barrier.

29. The method of claim 18, wherein the genetic vector encodes a chimeric polypeptide comprising:
a. a leader sequence which enables or promotes secretion of the chimeric polypeptide at neuronal termini located within CNS tissue that is protected by the blood-brain barrier of an animal; and,
b. a neuroactive sequence which can create a therapeutic benefit by reacting with cells located wholly within CNS tissue that is protected by the blood-brain barrier, after secretion within the blood-brain barrier by an accessible neuron which straddles the blood-brain barrier.

30. A genetic vector designed to initiate and capable of initiating a neuronal process that will non-invasively deliver neuroactive polypeptide molecules into central nervous system tissue protected by a blood-brain barrier in a higher animal, said genetic vector comprising cell transfection components and associated genetic material, which have been assembled into a genetic vector which is suited for and capable of transfecting at least one type of accessible neuron which straddles the blood-brain barrier and has at least one peripheral projection that is accessible to genetic vectors that have not penetrated an animal's blood-brain barrier,
wherein:
a. the cell transfection components are selected to cause the genetic vector to (i) bind to the selected type of accessible neuron, and (ii) insert associated genetic material carried by the genetic vector into the accessible neurons; and,
b. the associated genetic material contains at least one gene which encodes a neuroactive polypeptide, wherein said gene has a gene promoter that causes the gene to be expressed, inside transfected accessible neurons, into said neuroactive polypeptide or a precursor thereof, and wherein copies of the neuroactive polypeptide or precursor thereof have been shown to be (i) transported, within transfected accessible neurons, to neuronal secretion locations in central nervous system tissue that is protected by the blood-brain barrier of the animal, and (ii) secreted by transfected accessible neurons at such neuronal secretion locations, in central nervous system tissue that is protected by the blood-brain barrier of the animal.

31. The genetic vector of claim 30, which has been proven in in vivo mammalian tests to be fully capable of initiating a neuronal process that will non-invasively deliver neuroactive polypeptide molecules, encoded by the genetic vector and expressed within transfected accessible neurons, into central nervous system tissue protected by a blood-brain barrier in at least one mammalian species.

32. The genetic vector of claim 30, which has been proven in human clinical trials to be fully capable of initiating a neuronal process that will non-invasively deliver neuroactive polypeptide molecules, encoded by the genetic vector and expressed within transfected accessible neurons, into central nervous system tissue protected by a blood-brain barrier in treated human patients.

33. The genetic vector of claim 30, which is designed to transfect at least one type of sensory neuron.

34. The genetic vector of claim 33, which is designed to transfect olfactory receptor neurons.

35. The genetic vector of claim 33, which is designed to transfect nocioceptive neurons.

36. The genetic vector of claim 30, which is designed to transfect at least one type of motor neuron.

37. The genetic vector of claim 30, which is designed to transfect at least one type of pre-ganglionic neuron belonging to a sympathetic or parasympathetic nervous system of a higher animal.

38. The genetic vector of claim 30, wherein the gene encodes a neuroactive polypeptide selected from the group consisting of neurotrophic factors, endocrine factors, growth factors, paracrine factors, hypothalamic release factors, neurotransmitter polypeptides, antibodies and antibody fragments which bind to neurotrophic factors, antibodies and antibody fragments which bind to neurotrophic factor receptors, polypeptide antagonists, agonists for a receptor expressed by a CNS cell, and polypeptides involved in lysosomal storage disease.

39. The genetic vector of claim 30, wherein the cell transfection components comprise at least a portion of a mammalian viral capsid.

40. The genetic vector of claim 30, wherein the cell transfection components comprise a cationic liposome.

41. The genetic vector of claim 30, wherein the cell transfection components comprise a ligand which binds in a specific manner to at least one type of endocytotic receptor on the accessible neurons.

42. The genetic vector of claim 41, wherein the ligand has been identified through selection steps which include:
(a) subjecting a multiplicity of neurons of a selected type to contact by a phagemid or phage display library, each phage in the library carrying a candidate DNA sequence and having on an exposed phage surface a polypeptide encoded by the candidate DNA sequence, and the phage display library consisting of multiple phage particles with multiple candidate DNA sequences and corresponding polypeptides exposed on the phage surfaces;
(b) selecting at least one clonal phage line from the phage display library, wherein the selected clonal phage line was efficiently transported into neurons of the selected type.

43. The genetic vector of claim 30, wherein the cell transfection components comprise a polypeptide that has been shown to promote transneuronal passage of associated genetic material out of a selected type of accessible neuron which straddles the blood-brain barrier and into at least one type of neuron which is positioned entirely within central nervous system tissue and protected by the blood-brain barrier of an animal.

44. The genetic vector of claim 30, which encodes a chimeric polypeptide comprising:
a. a leader sequence which enables or promotes anterograde transport of the chimeric polypeptide within a neuron that expresses the chimeric polypeptide; and,
b. a neuroactive sequence which can create a therapeutic benefit by reacting with cells located wholly within CNS tissue that is protected by the blood-brain barrier, after secretion within the blood-brain barrier by an accessible neuron which straddles the blood-brain barrier.

45. The genetic vector of claim 30, which encodes a chimeric polypeptide comprising:
a. a leader sequence which enables or promotes secretion of the chimeric polypeptide at neuronal termini located within CNS tissue that is protected by the blood-brain barrier of an animal; and,
b. a neuroactive sequence which can create a therapeutic benefit by reacting with cells located wholly within CNS tissue that is protected by the blood-brain barrier, after secretion within the blood-brain barrier by an accessible neuron which straddles the blood-brain barrier.

46. A genetic vector designed to initiate and capable of initiating a neuronal process that will non-invasively deliver neuroactive polypeptide molecules into central nervous system tissue protected by a blood-brain barrier in a higher animal, wherein the genetic vector encodes a chimeric polypeptide comprising:
a. a leader sequence which enables or promotes at least one activity elected from the group consisting of:
(i) anterograde transport of the chimeric polypeptide within a neuron that expresses the chimeric polypeptide; and,
(ii) secretion of the chimeric polypeptide at neuronal termini located within CNS tissue that is protected by the blood-brain barrier of an animal; and,
b. a neuroactive sequence which can create a therapeutic benefit by reacting with cells located wholly within CNS tissue that is protected by the blood-brain barrier, after secretion within the blood-brain barrier by a neuron which straddles the blood-brain barrier.

47. The genetic vector of claim 46, wherein the neuroactive sequence of the polypeptide is selected from the group consisting of neurotrophic factors, endocrine factors, growth factors, paracrine factors, hypothalamic release factors, neurotransmitter polypeptides, antibodies and antibody fragments which bind to neurotrophic factors, antibodies and antibody fragments which bind to neurotrophic factor receptors, polypeptide antagonists, agonists for a receptor expressed by a CNS cell, and polypeptides involved in lysosomal storage disease.
